SEQUENCE LISTING (42 sequences)

SEQ ID NO: 1 - DNA, 7513 bp, Artificial Sequence
  Nucleotide sequence of cDNA copy of TRSV RNA1 (GenBank KJ556849)
ttgaaaattctctcacaaggttcccgttactttcaaaactctcttttcttcttataattgtctactgaatgtgattttgttttgtttcaacttgtgtaaagatgggctttacttgcccaaactccgattgtctctactcgcgtagcgagtggagcaatcgtgccctccgcgaggagggtcttacttttagcatgcgttgtcctggcgtatgctgcggagcactgttggtccgcaagcaacaggaacctgaagctgtggatagcgccacagcaccccgaaagagggtggatagcgccaccccaaaatgtttgtgctggcttgccagcgtggggccttccaagtgccccaaacactcccaggtgcctctggcaccaaaatccaaacctacgcccacggcggttgtagtttctgcttctcctctcaagaagcagagctgcgacgttgtggtcgctgttggcccaccggccaacctagagctggtttatccggctctagtctctcttggtgctgctgccctacctaaggcggagaaaaagagcttcaatgaggctcttttggagaagcgtgcggcctaccaggcgcgcaccgctgtaccaccacccgggcctattcgggtggtgaagaccgttgctgccccggttaaggcagaaaaggtgaagttcccaaaaggggctgtcgccttcaatggcatcaattttgttgatgctaagggccatgtggtcctgagcgctggagcgcttaaaattctgcgcggagctaaaaaactccggcaacagcaggcgcgatctatgcgccgcatggctgcctgccgccgtgtgcggttggcagcttttgctgctagagtgccttcccttttgaggaaggcagatgaagccacatctggtggctttaaatatgttgatttaaatgcaccaagggcggtgcgtgaggccgagaagcggccaaaaaagaagcccgccaagaaggcggccaggtctgcctcccctgttgaggaggaaattaactgggatgattttatcattccagattctgagaggactgcctccccgatgaaggaggaaaaaccaaagcgccctttggttcccaattctttgggtttcggctggtggcgtccagcttctggaaatctttgggacgctgtgtcccagtgtcagcgtgcctgtaagggcacctttcttgaagtctctgctgaggctaagctggtgtgtgctggcaccgatgatggagccctatctgtttgggccagaatttcaaagagtgttgtgcaactttctgcttattatgatgccaacactcttctggataattataatgcactgtctgagtgtaccatggatgaattacaatctgtggcagtgcagttggattctgagtaccaggaacttggaccacctacgcattttacgtgtgggttgtccaactgggctcgtggggctggcaaaattctctataattttgtagctcctactgtggaagggatagcaggcgcaggatgtcgtattgttgagcgcgcctatgagttgtctaaagtcgttattgacgagatcttcagtaaaatgaagtcccttttttatgactgctttggcaatttatttggccatttgaatgtgctgctatccacaattgatagtttttgggcccgtgcgtctacatggataatgaatatcttagagaaaacgcatgattgccttaaggtgcttagggacagtgctgtttggtccctattgttgattctggttggaggtttaatacttctttctgaaaggttcctgcaatccattggtataatttcaaagccagggaccattttgggtattttccttgcaacctttcttggcatatttgggtacacctttttccgtaaggacgacactttagtgtcagatcttttgtgtgcctttaaaattgctattacaaaccttttccggacaaagcctggtcctccaggatctcccatcattgtggacggtgatgtggtgataccggaaagtgctgtcgaaatgtccacatgtagtttcatgggcggtcttgatattgcaattgctgctattggaaatgttggagcttctatccttggttttaaggttggagctttacaatatgcagccaagatcgctacatgtctagaccagttgcgcaagggtaaagacgtgcttaaagagatgacctgctggatcattgaaacccttggcgccctgtggaataaaatcactggtcgtgaagccactttcttcgacgaagtttctgctattgtagcagttgatattagggagtggcttgaagaatcccaaaacttatgtctcgctgcgcaaactttttccatcggagataagattgttcttgaacaatgtgaacgtcttattgctgatggccacaaacttttgcggggcatgggtgatgccgatcggaagctttctagttctttcctttcaactgtacaaaggaaagtttctgatcttgagaaaatacatactcagtctgtccgtgctgggtattttgaaggaaggaggatggaacccttttgggtgtacatccacggaccctcccattgtggcaagtctctcttaatggaacccatgtctagggaactattgagagcgggaggtttttctgaatcgtctatctatacaaagaattcttgtgataaatactggtcaaggtatagaagacaagcttgtgtacagattgatgacttgtcagctggcaaaacggatccatctttagagagtcagctcatcaatcttgttgcttcaaaggaggtgccacttgatatggctgaagttgaagacaagggcatattattcgattcagccattcttgtcacctcctctaatacagcccacgtgccgacgaatgctaacgtcaaccatgcagaagcttacaagaaccgcatgaatgttgttatccaatgtaggaggaagcctgagtactccaacataggtgtcgagcttgaaggcacgtttcaaccatttgacccgcggaatccacaagcttcaattgagtgcatgttacaacaccgtgaaacgcatgcgcccattaccggatggatttctgctggggctgccatggctgaggcagtgaaccagttccgcttgcacagagagaaggagatgatcttacaaagtaatcatctttcctccttccgccctgcgcaccccatttacactgagtgtgccacttttttaagtatgtacgcgcgagacgcaagttttgtgccacctgtggaccttggttgtaaatgggaaattccaagtgggtacatgaccattgctgctgtggatggtcgcgtttttggtttcacacagcttggagtgtgtactgagataactaaacagttaaagttcactgacgaaatggaacagtacacactcgataaatttgccccagatattacgaagacaatggcttcccaaagtcgttttaagcttgttggggcatttttgaaaggaatgattagggaggaagacaatgttatttcactcacctccctagggcccaagagtactgcaactcaaagagaattttatgaaactcttgggcttgctgagcgggtttacctccgtgctgtgcaaaagaaagtcaataaaattcgcacggaccccgcttttgatgttgaggcgttgcatgctagactactgagcaacattgcaacttcctatgagtatgttaggacacatggacctaaaattttccctttactcatgggatttgtttgtgtagtctttgcatgctatggttttattatgcccctgctttcttttgcctcagggggctctgctgtaggtgggatggttgcaatggaacaaatgtctgcagcctctgttgtttcttctggatcaagtccagttgcccaccgcaaccgtgcaccaccagtgcaaccaagatatgctaggcatcgtttggctggagcttcagccgatgatgcctatgcttatgaagaaatgatggtggtgttgtacgttgactcgaccgttgctccggtagtcaatgctgttagagggcctggccgttcaattttcataacccggcaccaggccctcatgattcccaacaatagtaccgttgtggctcatttttccacacgtgatgttgttgagatccactgggagcacgacgtcgtcaggaaaggcgagaagaaggataccgaaattgttcaatatcgctgtccttctattcctgaacttccctctcgctgtaagaaatattttgaatatgatttagagagggatttccctggaccatttactttagatgccagctgttatagaatgcaaagtccagggaaaatcgatatagagctggtgagctggaccgaccatgatgcggaactccgcactcgtcctcttgttatcgctgatccatttggagaggacagatatagaagggaaattccccgatatatccagtatggtaggccagcgcaactccatgattgtggtgctatctgtgttgccaaaattggaggccaacatagaattgtgggcctagttatttccacagataagcacaacactggagttggtctgctgccgtcggcgcttcatatgataacttgttcccttacctatgtgcctgaggaatgggaagatgcgccacggggtttgaagaaattgggttggaagcacgcttctgaacttccacatatgccgcggaaaacccaatacgttgctgttaatgaggatcttgcaattccatttgataaccccaagataccaagtgtcttggtcccagatgatcctcgtactgtaggcacgcccgttgaaggtaaagatcctgtcttggttgcaatggaaaaattttatgaaccaatgacagactttacagaagaggaggtccgcccaggtcagaccgaggttagcttgtttgagcaagtgtgtgatgatattgtgcaaacctggtttgatgctggtgcggaatttgaagatgttgaagatgatgtcgtgatcaatggtgatgatgactttgataaattgatcatggacacatctgaaggttatccctatgtgcttgaaaggacgcatggggaaaaaggaaaaactcgatatttcgagggtggacctggggcctacactctaaagcccggtacttccgtgtataatgattatcatagactacaagaggaggtacaggttgaagggggtatccctgagatggtctgtattgagtgcccaaaagacgaattgcttgtggagcgtaaggttctgcaaaaacttggcactcgaaacttcgagatattggaattgcccaaaaacatgcttttcagaaagaaatttttgcattgggctttattcctttcagatatgcgatggtgcctgccgtgtcaagtgggcatcgttgttcaggggcgtgaatgggggctcctgatggatcgtcttgctgcgaagaattcggttgcttacaactgtgattattctaagtttgatggtctcatgtcttgtcaggtgttggatgctatagggaaaatggtaaataaatgctattccaacgccaaccccaatttaaagaagaaggggaagggtgagctgcccggtagtcctccccaattagctaggcataatttgttaatgtccatttttggtagaaagtgcttagctagatcccaagtttttgaagttcggggtgggatcccgtcggggtgtgcacttaccgtattgttaaactctgtatttaatgaaattctcatacggtatgtgtataaaacagtaattccatcaccagagtttaatcgctttgaaacttttgtgactctggtggtatatggtgatgataacctcattgcagttgatccttcaatgcaaaaaatttttactggtgaggttattaagaagacattggcgaggaagaaaatcactattactgatggtagcgataagttatctcctgtgctagaggcaaaaccactcgcgcagttagattttcttaagcgttccttcctcatttctgattcggggcaagtcatgccagcgttagatagaacttgcatatattcatctttgctatatctacgctcggctgattgtgatccaatacctctcttgcaccaaaatgtgcaaaatgctttgcaagagctgtattaccgccaagatcgggacgagtttgataatctccgtattttttatctcgagagattaccaatgtggcggaatggtcaacaccggctcctcgattggaaccaatgtggtgaacactggcgggcgcgttatactgggtgtccttccgataatcctgctggcgttctcgatatgttgattgatccaaggtgtaagagctttattcttcctgctggtccggctaattggtccatgccaatagcagaccgtatctttgtttgtgggcctaaattttgtccaagtgggtcctcatacaccttgtgtttcaatcgtcttgctgcaggtgagacaggtgtacaaataaaacctgttcatgctgcaactcagggagctatgcctactgctaagtttgtcgagagcttccgctccattaagaagagacctgagcttgaattagctatctctgcttatgagagtggaagtaatctttatttcaaaggctgtgctccttacaatgacatctgggcttgcgctatatctttttgctctgcttttggatatgcccagaaacaagtgctcctccacatgtatgataattgcaagcctttaggggctagttctttgaggagttactttaataaaaacctggttggtgatggttgtgcgcgacgttgtgaaattcatgcaacctcagctattgccaggcaggtcgaacgccttcttccacaggttcaatgcaaacattgtgaatatgatcctgagtttgcatcaaaacctactacccaattgcgtaaatgcacggatcctggggtagatggaggtaaggcaatgtatatagttcgtggcctgggcagaactgcagccaagttggtttgttcagacatgtgtgatggacatctaatgtcctgtaatacaacttttgacaaaatggttgtggatctgttcaggcaatcttgtttttaaatgcctttagttaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacac

SEQ ID NO: 2 - DNA, 3928 bp, Artificial Sequence
  Nucleotide sequence of cDNA copy of TRSV RNA2 (GenBank KJ556850)
ttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcagggtctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagaccggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacac

SEQ ID NO: 3 - DNA, 8941 bp, Artificial Sequence
  Nucleotide sequence of T-DNA region of pNMD36170
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacaaggttcccgttactttcaaaactctcttttcttcttataattgtctactgaatgtgattttgttttgtttcaacttgtgtaaagatgggctttacttgcccaaactccgattgtctctactcgcgtagcgagtggagcaatcgtgccctccgcgaggagggtcttacttttagcatgcgttgtcctggcgtatgctgcggagcactgttggtccgcaagcaacaggaacctgaagctgtggatagcgccacagcaccccgaaagagggtggatagcgccaccccaaaatgtttgtgctggcttgccagcgtggggccttccaagtgccccaaacactcccaggtgcctctggcaccaaaatccaaacctacgcccacggcggttgtagtttctgcttctcctctcaagaagcagagctgcgacgttgtggtcgctgttggcccaccggccaacctagagctggtttatccggctctagtctctcttggtgctgctgccctacctaaggcggagaaaaagagcttcaatgaggctcttttggagaagcgtgcggcctaccaggcgcgcaccgctgtaccaccacccgggcctattcgggtggtgaagaccgttgctgccccggttaaggcagaaaaggtgaagttcccaaaaggggctgtcgccttcaatggcatcaattttgttgatgctaagggccatgtggtcctgagcgctggagcgcttaaaattctgcgcggagctaaaaaactccggcaacagcaggcgcgatctatgcgccgcatggctgcctgccgccgtgtgcggttggcagcttttgctgctagagtgccttcccttttgaggaaggcagatgaagccacatctggtggctttaaatatgttgatttaaatgcaccaagggcggtgcgtgaggccgagaagcggccaaaaaagaagcccgccaagaaggcggccaggtctgcctcccctgttgaggaggaaattaactgggatgattttatcattccagattctgagaggactgcctccccgatgaaggaggaaaaaccaaagcgccctttggttcccaattctttgggtttcggctggtggcgtccagcttctggaaatctttgggacgctgtgtcccagtgtcagcgtgcctgtaagggcacctttcttgaagtctctgctgaggctaagctggtgtgtgctggcaccgatgatggagccctatctgtttgggccagaatttcaaagagtgttgtgcaactttctgcttattatgatgccaacactcttctggataattataatgcactgtctgagtgtaccatggatgaattacaatctgtggcagtgcagttggattctgagtaccaggaacttggaccacctacgcattttacgtgtgggttgtccaactgggctcgtggggctggcaaaattctctataattttgtagctcctactgtggaagggatagcaggcgcaggatgtcgtattgttgagcgcgcctatgagttgtctaaagtcgttattgacgagatcttcagtaaaatgaagtcccttttttatgactgctttggcaatttatttggccatttgaatgtgctgctatccacaattgatagtttttgggcccgtgcgtctacatggataatgaatatcttagagaaaacgcatgattgccttaaggtgcttagggacagtgctgtttggtccctattgttgattctggttggaggtttaatacttctttctgaaaggttcctgcaatccattggtataatttcaaagccagggaccattttgggtattttccttgcaacctttcttggcatatttgggtacacctttttccgtaaggacgacactttagtgtcagatcttttgtgtgcctttaaaattgctattacaaaccttttccggacaaagcctggtcctccaggatctcccatcattgtggacggtgatgtggtgataccggaaagtgctgtcgaaatgtccacatgtagtttcatgggcggtcttgatattgcaattgctgctattggaaatgttggagcttctatccttggttttaaggttggagctttacaatatgcagccaagatcgctacatgtctagaccagttgcgcaagggtaaagacgtgcttaaagagatgacctgctggatcattgaaacccttggcgccctgtggaataaaatcactggtcgtgaagccactttcttcgacgaagtttctgctattgtagcagttgatattagggagtggcttgaagaatcccaaaacttatgtctcgctgcgcaaactttttccatcggagataagattgttcttgaacaatgtgaacgtcttattgctgatggccacaaacttttgcggggcatgggtgatgccgatcggaagctttctagttctttcctttcaactgtacaaaggaaagtttctgatcttgagaaaatacatactcagtctgtccgtgctgggtattttgaaggaaggaggatggaacccttttgggtgtacatccacggaccctcccattgtggcaagtctctcttaatggaacccatgtctagggaactattgagagcgggaggtttttctgaatcgtctatctatacaaagaattcttgtgataaatactggtcaaggtatagaagacaagcttgtgtacagattgatgacttgtcagctggcaaaacggatccatctttagagagtcagctcatcaatcttgttgcttcaaaggaggtgccacttgatatggctgaagttgaagacaagggcatattattcgattcagccattcttgtcacctcctctaatacagcccacgtgccgacgaatgctaacgtcaaccatgcagaagcttacaagaaccgcatgaatgttgttatccaatgtaggaggaagcctgagtactccaacataggtgtcgagcttgaaggcacgtttcaaccatttgacccgcggaatccacaagcttcaattgagtgcatgttacaacaccgtgaaacgcatgcgcccattaccggatggatttctgctggggctgccatggctgaggcagtgaaccagttccgcttgcacagagagaaggagatgatcttacaaagtaatcatctttcctccttccgccctgcgcaccccatttacactgagtgtgccacttttttaagtatgtacgcgcgagacgcaagttttgtgccacctgtggaccttggttgtaaatgggaaattccaagtgggtacatgaccattgctgctgtggatggtcgcgtttttggtttcacacagcttggagtgtgtactgagataactaaacagttaaagttcactgacgaaatggaacagtacacactcgataaatttgccccagatattacgaagacaatggcttcccaaagtcgttttaagcttgttggggcatttttgaaaggaatgattagggaggaagacaatgttatttcactcacctccctagggcccaagagtactgcaactcaaagagaattttatgaaactcttgggcttgctgagcgggtttacctccgtgctgtgcaaaagaaagtcaataaaattcgcacggaccccgcttttgatgttgaggcgttgcatgctagactactgagcaacattgcaacttcctatgagtatgttaggacacatggacctaaaattttccctttactcatgggatttgtttgtgtagtctttgcatgctatggttttattatgcccctgctttcttttgcctcagggggctctgctgtaggtgggatggttgcaatggaacaaatgtctgcagcctctgttgtttcttctggatcaagtccagttgcccaccgcaaccgtgcaccaccagtgcaaccaagatatgctaggcatcgtttggctggagcttcagccgatgatgcctatgcttatgaagaaatgatggtggtgttgtacgttgactcgaccgttgctccggtagtcaatgctgttagagggcctggccgttcaattttcataacccggcaccaggccctcatgattcccaacaatagtaccgttgtggctcatttttccacacgtgatgttgttgagatccactgggagcacgacgtcgtcaggaaaggcgagaagaaggataccgaaattgttcaatatcgctgtccttctattcctgaacttccctctcgctgtaagaaatattttgaatatgatttagagagggatttccctggaccatttactttagatgccagctgttatagaatgcaaagtccagggaaaatcgatatagagctggtgagctggaccgaccatgatgcggaactccgcactcgtcctcttgttatcgctgatccatttggagaggacagatatagaagggaaattccccgatatatccagtatggtaggccagcgcaactccatgattgtggtgctatctgtgttgccaaaattggaggccaacatagaattgtgggcctagttatttccacagataagcacaacactggagttggtctgctgccgtcggcgcttcatatgataacttgttcccttacctatgtgcctgaggaatgggaagatgcgccacggggtttgaagaaattgggttggaagcacgcttctgaacttccacatatgccgcggaaaacccaatacgttgctgttaatgaggatcttgcaattccatttgataaccccaagataccaagtgtcttggtcccagatgatcctcgtactgtaggcacgcccgttgaaggtaaagatcctgtcttggttgcaatggaaaaattttatgaaccaatgacagactttacagaagaggaggtccgcccaggtcagaccgaggttagcttgtttgagcaagtgtgtgatgatattgtgcaaacctggtttgatgctggtgcggaatttgaagatgttgaagatgatgtcgtgatcaatggtgatgatgactttgataaattgatcatggacacatctgaaggttatccctatgtgcttgaaaggacgcatggggaaaaaggaaaaactcgatatttcgagggtggacctggggcctacactctaaagcccggtacttccgtgtataatgattatcatagactacaagaggaggtacaggttgaagggggtatccctgagatggtctgtattgagtgcccaaaagacgaattgcttgtggagcgtaaggttctgcaaaaacttggcactcgaaacttcgagatattggaattgcccaaaaacatgcttttcagaaagaaatttttgcattgggctttattcctttcagatatgcgatggtgcctgccgtgtcaagtgggcatcgttgttcaggggcgtgaatgggggctcctgatggatcgtcttgctgcgaagaattcggttgcttacaactgtgattattctaagtttgatggtctgatgtcttgtcaggtgttggatgctatagggaaaatggtaaataaatgctattccaacgccaaccccaatttaaagaagaaggggaagggtgagctgcccggtagtcctccccaattagctaggcataatttgttaatgtccatttttggtagaaagtgcttagctagatcccaagtttttgaagttcggggtgggatcccgtcggggtgtgcacttaccgtattgttaaactctgtatttaatgaaattctcatacggtatgtgtataaaacagtaattccatcaccagagtttaatcgctttgaaacttttgtgactctggtggtatatggtgatgataacctcattgcagttgatccttcaatgcaaaaaatttttactggtgaggttattaagaagacattggcgaggaagaaaatcactattactgatggtagcgataagttatctcctgtgctagaggcaaaaccactcgcgcagttagattttcttaagcgttccttcctcatttctgattcggggcaagtcatgccagcgttagatagaacttgcatatattcatctttgctatatctacgctcggctgattgtgatccaatacctctcttgcaccaaaatgtgcaaaatgctttgcaagagctgtattaccgccaagatcgggacgagtttgataatctccgtattttttatctcgagagattaccaatgtggcggaatggtcaacaccggctcctcgattggaaccaatgtggtgaacactggcgggcgcgttatactgggtgtccttccgataatcctgctggcgttctcgatatgttgattgatccaaggtgtaagagctttattcttcctgctggtccggctaattggtccatgccaatagcagaccgtatctttgtttgtgggcctaaattttgtccaagtgggtcctcatacaccttgtgtttcaatcgtcttgctgcaggtgagacaggtgtacaaataaaacctgttcatgctgcaactcagggagctatgcctactgctaagtttgtcgagagcttccgctccattaagaagaggcctgagcttgaattagctatctctgcttatgagagtggaagtaatctttatttcaaaggctgtgctccttacaatgacatctgggcttgcgctatatctttttgctctgcttttggatatgcccagaaacaagtgctcctccacatgtatgataattgcaagcctttaggggctagttctttgaggagttactttaataaaaacctggttggtgatggttgtgcgcgacgttgtgaaattcatgcaacctcagctattgccaggcaggtcgaacgccttcttccacaggttcaatgcaaacattgtgaatatgatcctgagtttgcatcaaaacctactacccaattgcgtaaatgcacggatcctggggtagatggaggtaaggcaatgtatatagttcgtggcctgggcagaactgcagccaagttggtttgttcagacatgtgtgatggacatctaatgtcctgtaatacaacttttgacaaaatggttgtggatctgttcaggcaatcttgtttttaaatgcctttagttaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaatctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 4 - DNA, 5356 bp, Artificial Sequence
  Nucleotide sequence of T-DNA region of pNMD36180
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaatctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 5 - DNA, 5505 bp, Artificial Sequence
  Nucleotide sequence of T-DNA region of pNMD43050
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaagagacctcgcgatttaaatcgatggtctcaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 6 - DNA, 1761 bp, Artificial Sequence
  Nucleotide sequence of Nicotiana benthamiana phytoene desaturase (NbPDS) cDNA (GenBank DQ469932.1)
atgccccaaatcggacttgtatctgctgttaatttgagagtccaaggtaattcagcttatctttggagctcgaggtcttcgttgggaactgaaagtcaagatgtttgcttgcaaaggaatttgttatgttttggtagtagcgactccatggggcataagttaaggattcgtactccaagtgccacgacccgaagattgacaaaggactttaatcctttaaaggtagtctgcattgattatccaagaccagagctagacaatacagttaactatttggaggcggcgttattatcatcatcgtttcgtacttcctcacgcccaactaaaccattggagattgttattgctggtgcaggtttgggtggtttgtctacagcaaaatatctggcagatgctggtcacaaaccgatattgctggaggcaagagatgtcctaggtgggaaggtagctgcatggaaagatgatgatggagattggtacgagactgggttgcacatattctttggggcttacccaaatatgcagaacctgtttggagaactagggattgatgatcggttgcagtggaaggaacattcaatgatatttgcgatgcctaacaagccaggggagttcagccgctttgattttcctgaagctcttcctgcgccattaaatggaattttggccatactaaagaacaacgaaatgcttacgtggcccgagaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgacgagctttcgatgcagtgcattttgattgctttgaacagatttcttcaggagaaacatggttcaaaaatggcctttttagatggtaaccctcctgagagactttgcatgccgattgtggaacatattgagtcaaaaggtggccaagtcagactaaactcacgaataaaaaagatcgagctgaatgaggatggaagtgtcaaatgttttatactgaataatggcagtacaattaaaggagatgcttttgtgtttgccactccagtggatatcttgaagcttcttttgcctgaagactggaaagagatcccatatttccaaaagttggagaagctagtgggagttcctgtgataaatgtccatatatggtttgacagaaaactgaagaacacatctgataatctgctcttcagcagaagcccgttgctcagtgtgtacgctgacatgtctgttacatgtaaggaatattacaaccccaatcagtctatgttggaattggtatttgcacccgcagaagagtggataaatcgtagtgactcagaaattattgatgctacaatgaaggaactagcgaagcttttccctgatgaaatttcggcagatcagagcaaagcaaaaatattgaagtatcatgttgtcaaaaccccaaggtctgtttataaaactgtgccaggttgtgaaccctgtcggcccttgcaaagatcccctatagagggtttttatttagctggtgactacacgaaacagaagtacttggcttcaatggaaggtgctgtcttatcaggaaagctttgtgcacaagctattgtacaggattacgagttacttcttggccggagccagaagatgttggcagaagcaagcgtagttagcatagtgaactaa

SEQ ID NO: 7 - DNA, 5689 bp, Artificial Sequence
  Nucleotide sequence of T-DNA region of pNMD42330
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaatgcagaacctgtttggagaactagggattgatgatcggttgcagtggaaggaacattcaatgatatttgcgatgcctaacaagccaggggagttcagccgctttgattttcctgaagctcttcctgcgccattaaatggaattttggccatactaaagaacaacgaaatgcttacgtggcccgagaaagtcaaatttgctattggactcttgccggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 8 - DNA, 5683 bp, Artificial Sequence
  Nucleotide sequence of T-DNA region of pNMD43741
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaataactggaaagggattccatatttccagagattggataaattagttggcgtcccagtcataaatgttcacatatggtttgacagaaaactgaagaacacatatgatcaccttctctttagcagaagtccccttctgagtgtatatgctgacatgtcagtaacttgcaaggaatattatagcccaaaccagtcaatgttagagttggttggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 9 - DNA, 2322 bp, Artificial Sequence
  Nucleotide sequence of soybean phytoene desaturase (GmPDS1) cDNA (NM_001249840.2)
cgacactactctcttctcttctcttctcttctacgtactgccgtggtgctttcaccactgcttaccactaaccttcctctctctctctgccgctgcaagcttggtactctcaactcaattctccaccttattcttttcacttcttcagctcttgttttttcccaaatctactttcaaagtgcctgaattctgcaacagtaatattaacactcctctcttttgttcaggctttatttccccaatggccgcttgtggctatatatctgctgccaacttcaattatctcgttggcgccagaaacatatccaaattcgcttcttcagacgccacaatttcgttttcatttggcgggagcgactcaatgggtcttactttgcgacccgctccgattcgtgctcctaagaggaaccatttctctcccttgcgtgtcgtttgcgtcgattatccacgcccggagctcgaaaacaccgttaatttcgttgaagctgcttacttgtcttccacctttcgtgcttctccgcgtcctctaaaacccttgaacatcgttattgccggtgcaggattggctggtttatcaactgcaaaatatttggctgatgctgggcataaacctatattgctggaagcaagagacgttctaggtggaaaggttgctgcatggaaagacaaggatggagactggtacgagacaggcctacacatcttttttggggcttaccctaatgtgcagaacctttttggagaacttggcattaatgatcggttacaatggaaagagcattctatgatttttgctatgccaaataagcctggagagtttagtcgatttgattttcctgaagttcttccctccccattgaatggaatatgggcaatattgaggaacaatgagatgcttacatggccagagaaagtaaaatttgcaattgggcttctcccagctatgcttggcggacagccatatgttgaggctcaagatggtctttctgttcaagaatggatgaaaaagcagggcgtacctgaacgggtaactgatgaggtgttcatagcaatgtctaaggcactaaacttcatcaatcctgatgaactttcaatgcaatgtatattgattgctttaaaccgatttcttcaggagaaacatggttctaagatggcctttttggatggcaatccacccgaaagactttgtatgccgatagttgatcatattcagtccttgggtggtgaagttcatctaaattcgcgcattcaaaaaattgagctaaatgatgatggaacggtgaagagcttcttactaaataatgggaaagtgatggaaggggatgcttatgtgtttgcaactccagtggatattctgaagcttcttctaccagataactggaaagggattccatatttccagagattggataaattagttggcgtcccagtcataaatgttcacatatggtttgacagaaaactgaagaacacatatgatcaccttctctttagcagaagtccccttctgagtgtatatgctgacatgtcagtaacttgcaaggaatattatagcccaaaccagtcaatgttagagttggtttttgcaccagccgaagaatggatttcacgtagtgacgatgatattattcaagccacgatgactgagcttgccaaactctttcctgatgaaatttctgcagaccaaagcaaagctaagattctcaagtaccatgttgttaaaacaccaaggtcggtttacaaaactgttccaaattgtgaaccttgtcgacccattcaaagatctcctatagaaggtttctatttagctggagattacacaaaacaaaaatatttagcttcaatggaaggcgctgttctttctgggaagctttgtgcacaggctattgtacaggattctgagctactagctactcggggccagaaaagaatggctaaagcaagtgttgtgtaacaaaaacaagaattgaaagagtcatggtagagtacaggagcatcatttcaactttggcattctttgtctgtggtcaggactcaggagaccttcaactttattagttcatacgaataaagaaaggctcagcttctgaaatttagctcaccgtcgtcaactgtgtgcaataagctatacggaacaaacgacatgtgtcaacttaaagtcagcccattgttttgttatcctccaattttctggatcaatgtttgtattggaaagaaatatgtcattattcaaacttgtttatatccactttttttatttatcaacatttgtcacaacctttcgttgagtagcaaattattcccagaaaggcacattacata

SEQ ID NO: 10 - DNA, 5611 bp, Artificial Sequence
  Nucleotide sequence of T-DNA region of pNMD45660
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttagtagggatcctctcgttgttgttttagagctagaaatagcaagttaaaataaggctagtccgttatcaacttgaaaaagtggcaccgagtcggtgctttttttctagacccagctttcttgtacaaagttggcattaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 11 - DNA, 4965 bp, Artificial Sequence
  Nucleotide sequence (genomic) of Flowering Locus T 2a (FT2a) gene from soybean (Glyma16g26660)
gagtatataagaaagcataagccaaattttgagtaaactagtgtgcacactatcccatgcctagtggaagtagggatcctctcgttgttgggggagtaattggggatgtattggatccttttgaatattctattcctatgagggttacctacaataacagagatgtcagcaatggatgtgaattcaaaccctcacaagttgtcaaccaaccaagggtaaatatcggtggtgatgacctcaggaacttctatactttggtaactcattaattttgtccaagtactctttttgttgttcatatttatagtgattttaaattgtagtagtaattttttataccaagctagaataatttttgtgagtttttctattgaaaatataaaactttttttaatggagaaactctcaaaaactcttttaactttgtattcaaacaatctcataattattagtaaaaaatttaaatatgattttagtttcattaaaatgagcaaattttatttttaatatttgtaaaaagaaattgattttctttttctttttctaagaatatgacgttattgtttttgaccatccatactactattatatgatatagcatccattaattgtttgtgtttaagggaaggtcaagtaccctctcaagtggacatgtacatacatgtgaggacacctttataatattttctacatcccaaaataaatagtattatttatctatctctttttaatgtattatttatgtatcttaaagtaagttagggattgaaaatattgatatcttattttaaaggaatgaaaatcaatcattttagaaaaattaaaactaaaaatttgctcattgcagaaagtaaagtaaagaaaaccccaccactcattttataggacttaaagtcatacttaaaccttactaaaatttcaagaattgaaatacggatacaagcagttaatgcaatatattcttagtgcaatcgaggatcattagatgttacaatatagtagtagtttctagcttaatccaacttgatttcatttcattttattaacagattgcggttgatcccgatgcacctagcccaagtgaccccaatttgagagaatacctccattggtgagccacaaatttttgtttaaacacgccatgaatgcaaacatatatatgaactgaactatacatttatgagtttttctagcttaatgttgtaatattatacccctatcttctctcaatgtgtcaggttggtgactgatatcccagcaacaacaggggctagtttcggtacgtatatgatgtttttccatttttaaaacctataattttctgtgcttaaagttaaacctattcaaaccattatttttaatttaaaattagtgagttttatttatgtgcatatactgataaaaaaaattagttcacatattgtatttttatgtatgaagtctattactggcttggtctgaacaagtaaataatcatatatcaaaacaagaaaaaatattttaaattaaagtataattgaaccaagtaaaatgcatgctattagttaccaaactaataagcacaaaaaataaaataaaaactgatagaaacatgattgattacttattcaaattcaccctcgcttgtattctgtgtcaaacgcacaatgtttgcttgctagctgtttaatttctttaacaaatggacacatatgctaagtgtattagtgatttatgatttctaattatttaaattgataggaactcagcttttcaaaaacattattattaattatcttaactgacgaaggtattagaaattttattgaacactgattcctaaactcgtaagttttggaatcaagtgtgttatgtgataataagaagatccccattaataattattttactgagtatatatagtggaagggatttaaattatggatattctacaaggttccatcaactttatcttaaaagtttatcttttgtcctttggagcagtacatctcctttccaacaaatgaatgatgttcgagtttatcaaaatataaatgtggtccaaaatattttaaaataatttttatttttttgatataataaatattcttttttattgagtaccttttttaatttatatctataattgttagaattattatttttatttaattatattagagaaaaatgatttagttaattaagagttaaatctccagtaatacaccccccccccccccccccaaaaaaaaaagtagcttaattttaattcattggctaacacaatgtgaaatactaatgttatgttgaaacggtgcatgcatgatgtatcaaagcatgcgttatgcatggatgtaggaatgctaaatgtggactgtgttttccattgactagtaatttgagactaatcctatgattatggtcaggcttagtgactaattaaaaagactctcaacaagtaccttcttcaaggttcgaattttgttttatttttaatttttattgccagagaatccagcttacaatcacgccaacctttaaaatgaagaacagatagagataagtatatgatactagggcatcatattgtaaagtacgatcagcaaacctgatgatgaccaacatacaatcccaattaaaatcggagattattatgatagatatttaagttgtttctttgtttttttttttataaaaaaaatctttatacaaaaccaaatatgcatatatatatatatatatatatatatatatatatatatatatatatatatatatatatatatatatacagttagttatacatgtgtataaatacacacaaacatacatttatttttatatatacatacaatatatagcagatactcaatattgaaaattaatttttgttttgatgtttcaaaaacgtttttattggttttaatcttcatatacaaatattatttttttaaatgcttctgtcattactttgtgataatatatgacatgcatctatatatgttacatcaacaaccatccttaaataacatgtaatgtaacatatatagtcatcgatgcatattacacattatcatgagtgaagatacaggtcatttaaaaaaaaatcaaatttatgaggattaaaactgacaaaacaaaaattgatagacataggttacattaattaagccatatatatttacaagaattaagaatatatatgttttacctgattattaagattcaaaatatgcaaccaaattagccaaattatgaaccatataagcattgaagtcttgtttaatttctctcttccatcagaactcaagtaagaatatatgtagccatccgaattcttaaagtgcatgcatgatcatgtgcaaaaagatgagaagattcattcagttcaagaaaaaagacacaaagcaactactgtgactaaaggagaatattcctattgaataaactaccttttgttggcaagatagctctaaaaccttcataggatttacagagttatagaacatcatagaaaaagcttcataacttgttgaagcaaatttaaataaggggagttaaaagaataagaattagatttgtttaaactccatttccaaacatcttattgaccaaaactcagcatcatatataattcaataaattcatcaccttgacaaaatgtgttctttctaagtgaaaaagaaccctcctccatttaaactttcacttcaaccataccccatcaacctactcaccaaggtaagcaatcttaacatccttctcatctgaaataagaaaaattcctctgatttttttaaagaattttgagatccatcatctctagtgaacccccaatctttccataaaaaaagaaaaaaaagaaaaatattctgcttccatcatctatctgctttttaaaaccagagcttaccagtatgtatgagtgtttctggctttctgccctttgcagtacgaaatgctaagatatttccactgaattgaaattacgtgttcccatttcctatttaatttccttagtttcttatctcttcagttagaggatgacacgtacttaattaaaatcaactctatcttctgattgctattatatttttgaagtattattttttaatttggaagagagggaaaaggacacaagattaggtgatttggctcctttctaaaacgttaggacagcagtagtaggaccatacatgtctgaacattattaatacaataccttatctaagaagaaatatagtactattttatgtttgacaatcaaactgaaatatattatcattgcatgtatataaatcataagagtttaatgagcgtcaaagtagatcaagtagaaatcacatatatataacttttaatataattattataaaaattaataaatttattgatcatataaaattattttatattgtgggtattgtaacgataaatatgcattatttgcaaaggccatgaggttgtaacatatgaaagtccaagaccaatgatggggattcatcgtttggtgtttgtgttatttcgtcaactgggtagggagaccgtgtatgcaccaggatggcgccagaatttcaacactaaagaatttgctgaactttacaaccttggattgccagttgctgctgtctatttcaacattcagagggaatctggttctggtggaaggaggttatactaagaaaaagtactttatattattgaaaaaataaagtagtataagcttcgttgagggtttcagaaatattaattggcaatctcccacactctttagtagtaaatgagtgtttttcaacttaattaaactgagcatacagtgaaataaattgctagctcagttggtagcagcaagtactctgcatatacacataaatgaaactgaagcatctaggttcatttttcttatttgtattatcagttgaagaatgttaaagatatctgatatacgtaaagtggaaaatataactcgagcataagttaaagtga

SEQ ID NO: 12 - DNA, 20 bp, Artificial Sequence
  Nucleotide sequence of GmFT2a_SP1 gRNA (target sequence)
gtagggatcctctcgttgtt

SEQ ID NO: 13 - DNA, 117 bp, Artificial Sequence
  Nucleotide sequence of gRNA scaffold
gttttagagctagaaatagcaagttaaaataaggctagtccgttatcaacttgaaaaagtggcaccgagtcggtgctttttttctagacccagctttcttgtacaaagttggcatta

SEQ ID NO: 14 - DNA, 5614 bp, Artificial Sequence
  Nucleotide sequence of T-DNA region of pNMD45680
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaagacaatacagttaactatttgggttttagagctagaaatagcaagttaaaataaggctagtccgttatcaacttgaaaaagtggcaccgagtcggtgctttttttctagacccagctttcttgtacaaagttggcattaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 15 - DNA, 20 bp, Artificial Sequence
  Nucleotide sequence of NbPDS_Hpa1 gRNA (target sequence)
agacaatacagttaactatt

SEQ ID NO: 16 - DNA, 2046 bp, Artificial Sequence
  Nucleotide sequence of Nicotiana benthamiana phytoene desaturase (PDS) mRNA, complete cds
gttcaggggtatctttttgtgggtaacggccaaaccaccacaaattttcagttcccactcttaactctttcaacttcaacacaacaaattagtatttgcttttccttctttgcttatctagtgcataacgattttctacaactttagcatagtccacaacgtgaaacacaactccttggcggtttataccgaggcttaatttactgctatcttgttcaataaaatgccccaaattggacttgtttctgccgttaatttgagagtccaaggtaattcagcttatctttggagctcgaggtcttcgttgggaactgaaagtcaagatgtttgcttgcaaaggaatttgttatgttttggtagtagcgactccatggggcataagttaaggattcgtactccaagtgccacgacccgaagattgacaaaggactttaatcctttaaaggtagtctgcattgattatccaagaccagagctagacaatacagttaactatttggaggcggcgttattatcatcatcgtttcgtacttcctcacgcccaactaaaccattggagattgttattgctggtgcaggtttgggtggtttgtctacagcaaaatatctggcagatgctggtcacaaaccgatattgctggaggcaagagatgtcctaggtgggaaggtagctgcatggaaagatgatgatggagattggtacgagactgggttgcacatattctttggggcttacccaaatatgcagaacctgtttggagaactagggattgatgatcggttgcagtggaaggaacattcaatgatatttgcgatgcctaacaagccaggggagttcagccgctttgattttcctgaagctcttcctgcgccattaaatggaattttggccatactaaagaacaacgaaatgcttacgtggcccgagaaagtcaaatttgctattggactcttgccagcaatgcttggagggcaatcttatgttgaagctcaagacggtttaagtgttaaggactggatgagaaagcaaggtgtgcctgatagggtgacagatgaggtgttcattgccatgtcaaaggcacttaacttcataaaccctgacgagctttcgatgcagtgcattttgattgctttgaacagatttcttcaggagaaacatggttcaaaaatggcctttttagatggtaaccctcctgagagactttgcatgccgattgtggaacatattgagtcaaaaggtggccaagtcagactaaactcacgaataaaaaagatcgagctgaatgaggatggaagtgtcaaatgttttatactgaataatggcagtacaattaaaggagatgcttttgtgtttgccactccagtggatatcttgaagcttcttttgcctgaagactggaaagagatcccatatttccaaaagttggagaagctagtgggagttcctgtgataaatgtccatatatggtttgacagaaaactgaagaacacatctgataatctgctcttcagcagaagcccgttgctcagtgtgtacgctgacatgtctgttacatgtaaggaatattacaaccccaatcagtctatgttggaattggtatttgcacccgcagaagagtggataaatcgtagtgactcagaaattattgatgctacaatgaaggaactagcgaagcttttccctgatgaaatttcggcagatcagagcaaagcaaaaatattgaagtatcatgttgtcaaaaccccaaggtctgtttataaaactgtgccaggttgtgaaccctgtcggcccttgcaaagatcccctatagagggtttttatttagctggtgactacacgaaacagaagtacttggcttcaatggaaggtgctgtcttatcaggaaagctttgtgccgaagctattgtacaggattacgagttacttcttggccggagccagaagatgttggcagaagcaagcgtagttagcatagtgaactaaaatgttaattctgtacacaaaatttaagatgaaggcggccacgctgaattagcgttgtacac

SEQ ID NO: 17 - DNA, 5916 bp, Artificial Sequence
  Nucleotide sequence of T-DNA region of pNMD46661
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttaagaaatctcaaaattccggcagaacaattttgaatctcgatccgtagaaaccagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagacacgaagtagtgtgtgagacaatacagttaactattgttttagagctagaaatagcaagttaaaataaggctagtccgttatcaacttgaaaaagtggcaccgagtcggtgctttttttctagacccagctttcttgtacaaagttggcattaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 18 - DNA, 305 bp, Artificial Sequence
  Nucleotide sequence of Arabidopsis U6 promoter
agaaatctcaaaattccggcagaacaattttgaatctcgatccgtagaaaccagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagacacgaagtagtgtgtg

SEQ ID NO: 19 - DNA, 5611 bp, Artificial Sequence
  Nucleotide sequence of T-DNA region of pNMD47681
tggcatgcacatacaaatggacgaacggataaaccttttcacgcccttttaaatatccgattattctaataaacgctcttttctcttaggtttacccgccaatatatcctgtcaaacactgatagtttaaaccacttcgtgcagaagacaatagtggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactttgaaaattctctcacagggttccggttacgttgttcttttactctctttttattttaattgttcaaaattgcgattctgaagcttcctactttctggtgcccgcattttctctctgctccgatggagcccctcttatggcacgttgatgccaccactccttcccacatccaggctctccagtcaggctctctccctcctgcttctccggccgcggctctcacccgcgtccaacgcgccctttccttcttccggactgctgcccggaagtactgcaagcaggccgatgcgcctgacttgtttgccctggcaatgaccagggttgctgagcacaacgacattgtcgttgatgcaagaaatgtggaacagctgttccactttgttggccaacatgtggccaacactgctgagcgcaaggcgctccgggctgctcttcgtgagcagcgggctttatttaaggcatccctgcccggtgcctgttttcctgctccagctcctccaggctggggtattccaaaacccccccctcttcctccaccttttgtgtggaaaggatgccgctacaatgtggtggcccctcctccacgcatcccgcaaccacctccactgcctaaatttgcgccttttgtgcgcaacaactttagagtggttgctccccctccccttggtgaggtgtaccaacccgttggtgcacctttcccacagacccgggcctccgcggccctctccttctttcgcactgcctccacctgcaggcaggtcttggttgagagctgcattcagcagcccgccttcatgacttgctgtgcttctaccggggaagtgcaagaaatgacgagcatgcttactgaagctcgtcagactggaaaaatcctgactcccaaagaggtgagtcaggccttggcccaaaaacgcaaagaaatcaaaggggctgaggaaaatcgcatctcctttgatgaaggggtgcatttgacagaggcggatgtcttccatcgtctcagtcttgcgaagcgcttcatggcgcataagcgagatcggactttggtggacgttttaatgcctactgaacatgaagttgttcggtatccaggcacccgccctgacgggacattgcagatgtgcgtgtctgcccttccacgcatgtctgaggaagcagctaggaagctgcttgagaaggggtggaagaactccaaaaacgtctctctagacattggggttacttcctatatgccatatggtgcgcccatagttgcattcatgactattatggatgggcgtaccgatgatccacaagaggcagcactttgtgcgaattacatggaccttggtcgagaaaagtccaaggtgttgtctcttccacttgttaccattcccctctctgagattgaacatgatcaaggcattttggattgcctttatattgttacatattttcatggtgttcaatcttatcaacccggaactttaatgatgagttatggaactcttgagtttcaagaatattccaataactcttttacaactgctactcgggttagagagagttgggaccagattctcaaacgcaatgagaatcttgggaaaagagtccatgccggcatcggggtgcttggcactattgaaaaagaaatggaccagcaacttgaggacttccccgccataaatctggaaactaggccacggcctgttgtgcgaacttttcagaatgcacatcagccgttgcataagaccagatctatgcgaattggcactacctcttttactgggaacactggtaggactgtactcccaccagtggttaaaacctatgaagagggaaatgctaattttgattccctacaatccaaaccacgtcacagctctgccagcactgctcatttgatgtgtgctgtgacggttgttcccgatcccacttgttgtgggacattgtcctttaaggttcccaaagatgcgaagaaaggaaagcatcttggaacttttgacattcggcaagccattatggaatatggtggtttgcattcccaagaatggtgtgcaaagggcattgttaatcccacttttacagtgaggatgcatgccccacgcaacgcctttgcaggtttgtctatagcgtgcacctttgatgattacaaacgcatagacttaccagcgcttgggaatgaatgtcctccctccgagatgtttgaactgcctaccaaggttttcatgcttaaagatgcagatgtgcatgaatggcagttcaactatggggaacttacaggacatgggttgtgcaattgggcaaatgtagttacccagcccacattgtacttttttgttgcgtccacaaatcaagtgacgatggctgctgattggcagtgtattgttactatgcatgtggacatggggcccgtcattgatcgttttgagttagttccaactatgacgtggcctattcaattgggtgacactttcgccattgatagatattatgaggcgaaagaaattaaacttgacgggtcaacctccatgttgtctatatcttataattttggaggtcccgtcaagcattctaagaaacatgccatttcatattcccgggcagttatgtctaggaatcttgggtggtctggcactataagcggaagtgtcaagagtgtttcttctttattttgtaccgcttcttttgttatttttccatgggaacatgaagcacctccaaccttacgtcaggtgttatggggcccacatcagataatgcacggagatggccaatttgaaattgctatcaaaactcgtcttcattcagctgctacaactgaagaagggtttggtagacttggtatactcccgctttctgggcctatagctcctgatgcacatgttggatcgtacgagtttattgtacatatagacacttggcgacccgactctcaggtgcatcctcccatgttttctagtgcggagctttataattggttcactttaaccaatttgaaaccagatgcgaacactggcgtagtcaactttgatattcccggatacatccatgacttcgcctctaaggacgcaactgtgacgctcgcatcaaatcccctctcttggcttgtcgcagctactggctggcattatggtgaggtggatctctgcatctcctggtcaaggtccaaacaggcccaggctcaggagggtagtgtttccattaccactaattatagagattggggtgcttactggcaaggccaggcccggatttatgatttgcggcgtactgaagcggaaattcccatcttcttgggttcttacgctggtgcgacgccatctggtgccttgggtaagcaaaactatgtccggatttcaattgtcaatgctaaggacatagttgcactgcgagtgtgtttgcgacccaaatctataaagttctggggtcgctccgccactttgttttaaatgcctttagttactggaagcaagagacgttctgttttagagctagaaatagcaagttaaaataaggctagtccgttatcaacttgaaaaagtggcaccgagtcggtgctttttttctagacccagctttcttgtacaaagttggcattaggtgttttgtcttttgattcttctaaccaggattggtagcccttctggcattcatttgctggaaaccatgctgtacatggttggttagatagaagcgtcaataaaccgggactttaattagttccgttttaggttcctgcccttactagggtggtccggtccttaggtggatcgggaagctgtataaactcagcttcttgggggtgagagcccaagaatgtctgtctgtcagctgcttgtgtaaatgagctttctcccaggatagctctcctgggcacaagtgaaaactactgtcgtggcgagttagtagccagacgaccggagtaaaatctctagttaacaaaatgtgttagaccgttgtttctggcagctttgttgggtctgttgagttttctaaagctgctttgctatttgctgttgtttgagcccactgttcatatttggtttggggccttttatgtttcctagtgtcgtgtttgtccaacacaaaaaaaaaaaaaaaaaaaaaaaaaaaaaagcactagtgtctgtacttatatcagtacactgacgagtccctaaaggacgaaacgggagaacgctagccaccaccaccaccaccacgtgtgaattacaggtgaccagctcgaatttcccctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcacgctaagcttgaattcttgcttgtcttcacagagtggggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 20 - DNA, 7127 bp, Artificial Sequence
  Nucleotide sequence (genomic) of phytoene desaturase gene GmPDS11 (Glyma.11G253000) from soybean
ctcaacacctcttatttatgtatatgtctttgttcataccacctcataaatcttattaaagttacatattttcttattttctctctccggatgtcttgtagcccaatggtatcaaacattttattgtgatttagtttgacgggggttgacactttgtcctaatcttcctcacaatccactcaggaatattcattcattcttaaaacacacctcagtcacacaacacaactctcgtctcttttatgtactgccgtggtgctttctccactgcctaaaccgactctctctctctgcctctgcaagcttgttacttggtactcttttcacttcttcaactcttgtttttttttatttcccacatcaactttcaaagtgcctgaattccgcaacagtaataccattaacacgtctctcttttgattcaggctttgtttgattgattgccccaatggccgcgtgtggctatatatctgctgccaacttcaatcatcttgttggcgccagaaacttatccaaattcagttcttcggatgccacaatttcgttatcatttggcgggagcgattcaatgggtcttagtttgcgacccgctccaattcgtgctcctaagaggaaccatttctctcccttgcgtgtcgtttgcgtcgattatcctcgaccggagctcgaaaacactgttaatttcgtcgaagctgcttacttgtcttccaccttccgtgcttctccgcgtccactaaaacccttgaacatcgttattgctggcgcaggtattaacctagccatgcctatgcttgtacccatttcgttttatgcatcaaaattgtattttttttatcgacaaatgctggttgttcgtattgttatttcgttttatgcatcaaaagtgtatctttgctgccaaatttggtttttcttgctcttgttgttctgggtgttttcaaattaggacctgttataccacccttttcatttcatgtatggtcttctattctaatgttagtgtgtgttagaaaacaacttagaagcctgcttgggctacgcattaggaattcctttctttgtgattaaatagatgtacacatctcaatgcttgattacattgtattgtgacagtcctcctgtgtcggtaaggaaaaaagttatggaattttaggatcttcaatttttgctgaaatttcaaataaaaggtatcctgatatcatgttggatatttaaagacaaaaaaatttgtaaactaatctttgcacattagttttggcctctctgcctattggattgatgcatgtttcattattgcaggattggctggtttatcaactgcaaaatatttggctgatgctgggcataaacctatattgctggaagcaagagacgttctaggtggaaaggttttcctgctaatttaatctcttacgtcaattagttgtcactttgtgtgcattctgctcattattttagcatgctgtttaatgaaataagaaatttgtttgtgttgttccgtcataatctatttgatgtgttggctttataacttcatcatcaggcttgttgaagattaccgcagtaatgtaaaacatatttataataaatcaattttgatgcattgtgtttttacactattgtttcattttctaaggttgctgcatggaaagacaaggatggagactggtatgagacaggtctacacatcttctgtaagttcattaaaatctcgggtttaactttttttttttcctgctatgcatccaaatagttaggattttgatccttgctaccccactcttctgattaagaattaaatttcagttaaaaggtttgggttaaattgtgctctgtccaagctcaaaaggcttttccttgagtgggcaatttggcctcctagcagtgctttcggatgagttagtccatggcacacttattgaaatattttgatatatctttttagtttttctttctaaatgaatcatttattggacatcatattatttaatcaatacagatcttttaattattagaatccttattttgtagttatctttgctttacaactttttccccctccccacagttggggcttaccctaatgtacagaacttatttggagaacttggcattaatgatcggttacaatggaaggagcattctatgatttttgctatgccaaataagcctggagagtttagtcgatttgattttcccgatgtccttcccgccccattaaatggtaagatgtaagacctactgtatgtgtgtatatttatatacctaactgcagttggatgtctctataaaaagcacattgaatgcctttatctttggaaatcctacaatgctgaatttgcagttgtctgacataaaatgtgtaataccatctagcaattatcctgcctatgttacgagtaatgctcgaaaaccagaattcagatgaagaaaattttagtttccttgaaaattttaacacaatttgctagtcaaatttttgatttcgaatatatctcaatgcatttgataatcattgaaacaagatgtgttaaattgtttccattttatgtaaattaaaaattgcaaacccaaacagagaagaagcattttggtgtttagcttggtttctatattcttttctatttctttcttcactaatctggtttgcaggttgggaacaaatttttctcttctaaaaagaaccaagtgatgcttaaattctccaattttattttaacatctgcatcattccccttttaagattctgtttcattatgctttactttctaaatgttaaccagtatttacgagttgcatgccatgaatgtcaatacaggaatatgggcaatattgaggaacaatgagatgctgacctggccagagaaagtcaaatttgcaattgggcttctaccagctatgcttggtggacagccatatgttgaggctcaagatggtctttcagttcaagaatggatgaaaaagcaggtatagcacttttcttgattgtgtaaccttagcagtccattaatagatttaagtaacttggctatatcaccagtcatgggctactgcaacttaaaaattgcagagaaaagtcttgtttgttgacaatggactgttgaccatctgatgactattctgaaatggggtgattcaggaatagactttcatgtgacttgatgatgatgacaatgggtccttttatcattagagaaaggatatctgtagttgtaattgtaagattgtattacttgatatttacttaatgctttaaaatgtatttcatctacctcgggttgatttcactgggacagaatggaaccttgtcatggtctgtgttgcaaataatttgaaaataacaaaatgttttctccttgttatagggcgtacctgagcgagtaaccgatgaggtgttcatagcaatgtcgaaggcactaaacttcatcaatcctgatgaactttcaatgcaatgtatattgattgctttaaaccgatttcttcaggtgcgaacatttccttttcttaaaacactattctactagaattattgtatgattgacaaggtcattttattcccattataatagttgaatggaatcagattagtaataaactttaagatggccttttatttcacaaacatgtttctggaacttaggatcaagtttttttcttttccttttctttttcttcaactaattgttatattattaaattattctataggagaaacatggttctaagatggcctttttggatggcaatccccctgaaagactttgtatgccgatagttgatcatattcagtccttgggtggtgaagttcatctaaattcgcgcattcaaaaaattgagctaaatgatgatggaacggtgaagagcttcttactaaataatgggaaagtgatggaaggagatgcttatgtgtttgcaacacctggtatttttcttcttattttcccaatgtattaattaattgttttcatgacattcttaatcttcataccttaacatttttgaggttctttttcttgaaaacagtggatattctgaagcttcttctaccagacaactggaaagggattccgtatttccagagactggataaattagttggagtcccagtcataaatgttcacatatggtaagtaatggcttttgacgtttaaaaccatatgcatgcatatgttagactccaattttaaattaaattattacagtcatcagtatttcatttttctgtctaatatataaaaattcaaatattcactgaaaagtttggaatagaaatctatgcgatttttcttaaattgtattaatgacaattgacaatttataattaatgttcttctaattgaatggttgtaatagttttggtaaatggaaactttaaatagagttgtggtaaaataatgtcactatcactaattgatgtaataatattctttgttaactttttcataaagaaattcctcttactttattaatgttttgtcaaaattttgggtaacaccaacaaaaataatttgtaatgtttgtggataatgattatattataatctccactgttgtggtgacatactatatatgcatgtgtttgcaggtttgacagaaaactgaagaacacatatgatcaccttctctttagcaggtcttccacaaactttgaaacacctgtaatgtctggaagggatagaaatgctgttcatttaatgtctgttttgtttttctgatgcgtagatgcatattgactactgagtcaacttttgtgcttcataagttgtgaaactcgacaagtataaaacagaaaggtttactaagggataagaacatttttcattataatattgtgaaataacaatcagaagccaaactgaatattgtaatatggaaatagtgtagaagatatcctctccctaaatgttggccctctaatgatgtgccaaactgaatattgtaatatggaaatagtgacctatctattactaacatgtgattacaggagagagacaccactttattgaggtttgtgacataccctaatcttggaatggagtattggtctcattcttctataatttactaaagaaagaaagtgataaaaggaattaaaaatttgtggccaaggagatcctttctcccattgtttgcttaacttggctgagtttgttcaagaatttattcagggtgttgatggtgtctagcatgaggtggtttattgtgactttgtgattctgtgtatatggtagtgtttctttcatctacaatttccaattgccaatgattacatctcgtcaatagccaatgataaaaggatttttttttctattgcaattgcaattgatacaatctagttatttgctggcgggtttttaattttattgtgtatgttccaagtgtctgaggattttagggatgaaagatcacttattttttgctgtgtgaaatgaactttttttcttctttgctttcttgatggtatttgtggattataaataaagtattttcatcacctcacatcttgtatattttatatgtttcttcttgcagaagtccccttctaagtgtatatgctgatatgtcagtaacttgcaaggtaaaaaccacttcttacatgccactgatttgcactgatttatttcctcaacccatgcaaaagttgtggcctgcatgtataggttagcatattcgttcttatatgtataattaactagggtcacctaaatgaattggagatggtgctttctggataagcagttaaccctctctacaaattatttcagtttccagacatttatgtaatattcttttgttgtttgaggctgaatttcttctgcttatgttttgtctgcttatcatcaaatgttggaatgttgtttgagttctgtttgacgatcaaatgcaggagtattatagcccaaaccagtctatgttagagttggtttttgcaccagccgaagaatggatttcacgtagtgatgaagatattattcaagccacgatggctgagcttgccaaactcttccctaatgaaatatctgcagaccaaagcaaagcaaagattctcaagtaccatgttgttaaaacaccaaggtttgattgaataaccagtctggcaatgtctttgttgttactgaactgatggtgacatttatagcccttacattcagctgtgttctgaatcctaatggaactactggctacattgtttcaggtcagtttacaaaactgttccaaattgtgaaccttgtcgtcccttacaaagatctcctttagaaggtttctatttagctggagattacacaaaacaaaaatatttagcttcaatggaaggcgctgttctttctgggaagctttgtgcacaggctattgtacaggtaaaatctgtcacaaaaatatctatgtaactcagcgattcattagcaagaaatgtgattatctatatataaatgctttcttagttattttgtgagagaaacttggttattaattcagcaggatgaataacactacattgtttcagcaggataaaaatctttccacttttattttttggttgaaacatttttactgatttccaattatcctcaaaacttatgctgtctctgaatgatttttatatgtatctccattacccccctgcgaaaggcctttggttatgaaacattatttgaaattagtatgaaagagttagcataatgaggattgatcttctgcatagtttgatttgaatgtttttcttatcagtgctgtcattgaggcttgctaaaaattattgcaggattctgagctactagctactcggggccagaaaagaatggctaaagcaagtgttgtgtaacaaaaacaggaattgaaaatgagtcatggtagaatacaggagcatcaattcaagattggtattctttatgtggtcaggactcaggagaataaaaaagaaaggctcaccgtcaactatgtgcaataagctatagggaacaaatgacatgtgtcgatacttgaaaattcagcgcattgttttgtatcctccaagttactggatcaatgtttgtattggaacgaaatatgccatcatttaaacttgtatatccagtaattttatatatcaagatttgtcacaacctttcgctgagtagctaattatt

SEQ ID NO: 21 - DNA, 8582 bp, Artificial Sequence
  Nucleotide sequence (genomic) of phytoene desaturase gene GmPDS18 (Glyma.18G003900) from soybean
ggtactagtcactgtactggccagagaggtaaacgttagtaatactaagacaggtaggtattattgtcgtttcattggacggggcttgacactttgtcgtaatcttcctcacaatccactcaggaatattcattcatttcttaaacgttaaaacacaccacacgacactactctcttctcttctcttctcttctacgtactgccgtggtgctttcaccactgcttaccactaaccttcctctctctctctgccgctgcaagcttggtactctcaactcaattctccaccttattcttttcacttcttcagctcttgttttttcccaaatctactttcaaagtgcctgaattctgcaacagtaatattaacactcctctcttttgttcaggctttatttccccaatggccgcttgtggctatatatctgctgccaacttcaattatctcgttggcgccagaaacatatccaaattcgcttcttcagacgccacaatttcgttttcatttggcgggagcgactcaatgggtcttactttgcgacccgctccgattcgtgctcctaagaggaaccatttctctcccttgcgtgtcgtttgcgtcgattatccacgcccggagctcgaaaacaccgttaatttcgttgaagctgcttacttgtcttccacctttcgtgcttctccgcgtcctctaaaacccttgaacatcgttattgccggtgcaggtaataaccaacctatgcctatgctcatacccatttcgtattatgcataaaaaattgtatctttgcttccaattttgtgttttttcttgctttagttgttctgggtgttttcagattaggactcgttatacccttttcatttcgtgcatggtcttctaatgttggtgtgtgttagaaaaaacttcgaagccagcttgggctacgcattaggaatttccttctttgtgattaaatagatgtacgcatctcaatgcttgattacattgtgttgtgacagtcctgtgtcggtaaggaaaaaagttatggaattttaggtaccctgatatcatgttggatatttaaagacaaactaatctttgcacattagttttggcctctctgtctattggattgatgcatgtttcaaattatcgcaggattggctggtttatcaactgcaaaatatttggctgatgctgggcataaacctatattgctggaagcaagagacgttctaggtggaaaggttttcctgctaatttaatccctttcggcaataagttgtcacttagtgcgccttcttttcattattttaacaagctgtttaatgaaataagaaatttgtttgtgttgttccatcataatctatttgatgcgttggctttataacttcatcatcaggtttgttgaaggttaccacagtatatacaacatatttataataaatcaattttaatgtattgtgtttttatactattgtttcattttctaaggttgctgcatggaaagacaaggatggagactggtacgagacaggcctacacatcttttgtaagttcattaaaatcttgggtttaactcttttttttttctgctatgcatccatttccttaacttgtttttttttcttaaaaaaattccgctttcattttaataatatcatgcctttgttgagccttttttttcctttgataaaagcggattgaaaaaagagtaaaatgctggaggatcaggaatatttctagagctactaaagaaaccactagagaataaaattaaagagataacaaaagggggggtgagaggtcgcaggtctaaattttccactaacatttctaacaaaactaacaaactaatatttttcgataaaaaaaaaagataaaacaaaggtcataattggtagagttgatccttgacactatcaaagcctttgccttcatgaccaaatagttaggattttgatccttgctaccccactcttctgattaagaattaaatttcagttaaaaggtttgggttatattgtgttttgtccaagttcaaaaggctatagcttgagtgggcaatttggcctgtcaatgggattgattagcagaatattattagttacgaattgacataaattgcaaaatatttgttattattggcacatatttgttgcattcaactttccacatgaaagaccttggtcaactcactcatctcttgaggttagaagtacactttcaacaaaaaggaatttttgtcaatcaacacaaatatattcaagatctaattcaattagttggtctcactaattatgctcctgttgaaacttcaatggaaattaatctgaaattgcgacgagatgaaggtgaccttctactagacctaactttctatcgtaagctggctgtaagtattatttacctaaccatcactagaccaggcatctcatttgctgtccacacagttagcaaattcatgcaatttcctcggcatttgcatctttcagcagtacactgtattattaagtatctacttggtacttccagctgtggttttttcttccttactggtgcatcaatacaacttcaagcatacagtgattttgattgggatggatgtccaaacacacagaaatccactactggttggtgtatgttcttaggggaagctccaatctcttggaaatgcaagaaacaagactcaatctccaaatcgtccactgaagcagaataccgtgccatgtctactgcatgctttgagattatttggcttcgcggtctcctttcagagcttgatttttcacaagcaaaacctgctccactgcatgctgacaatacaagtgtcattcaaattccacaaatcctgtttaccatgaaagaacgaagcacatagaggttgattgtcactcaattcgggaggcgtatgaccattgagttatcatcttgcctcatgtttctacatatgttcaactcgctaacattctcacaaaatccttgatacgtcagtgtcataatttcctagtcagcaaattgatgcttttagacttactagtatcaatttgagggggggtgtcaatgggattgattagcagaatattagttattaattgatacaaattgcagaatatttgttattggcacaaatatgttggcaattcaaaacagcagatcttacatgattatacagctgtaattatctcgttaattagttatcccagctataggatagcatttattgtagattgactatttaatgcatagatgtagaaatcaaattgtatgaacactatttaatgcagaattctcggagaacacagtttttcatctgcaactttcttagtctaatatggcctcctaacagagcttttggatgagttagtccatgccacacttattaaactactttgatatgtttttttagtttttctttctaaatgaatcatttattggaaatcatattatttaatcaatacagatctttcaattatcggaatccttatgttgcagttatctttgctttacaactttttccccctccccacagttggggcttaccctaatgtgcagaacctttttggagaacttggcattaatgatcggttacaatggaaagagcattctatgatttttgctatgccaaataagcctggagagtttagtcgatttgattttcctgaagttcttccctccccattgaatggtaagatgtaagacctattgtatgtgtgtatatttatatacctaactgaagttggatgtctctataaaaagtacattgaatgcctttatctttggaaatcctacaatgcagaatttgtagttgtctgccataaaatgtgtaatactatctagttattatcctgcctattttatgagtagtgctcgaaaaccagaattcagatgaagaaaattttagtttccttgaaaattttaacacgatttgctagtcaaattttgattttgaatatatctcaatgcatttgataatcattgaatcaaggtgtgttaaattgtttccattttatataaattaaaaattacaaacccaaacatagaagaagcattttggagtttagcttggtttctatattcttttctatttctttcttcactaatctggtttgcaggtcaggaacatattctcttctgaaaagaaccaactgatgcttaaattctccaattttatttttacatctgcatcattccccttttaagattctgttttattatgctttacgttttaaatgttaaccagtatttacaagttgcatgccatgaatgtgaatacaggaatatgggcaatattgaggaacaatgagatgcttacatggccagagaaagtaaaatttgcaattgggcttctcccagctatgcttggcggacagccatatgttgaggctcaagatggtctttctgttcaagaatggatgaaaaagcaggtatagcacttgtcttgattgtgtaaccttagcagtccattaatagatttaagtaacttggctatatcaccagtcatggcctactgcgacttaaaaattgcagagaaaagtcttgtttgtttgttatcattagagaaaggatatctatagttgttattgtaagatttcattacttgatatttacttgatgctttaaaatatatttcatctacctcaggttgatttcactgggacagaatggaaccttctcatggtctgttgcaaataattgaaaataacaaaatgttttctccttattatagggcgtacctgaacgggtaactgatgaggtgttcatagcaatgtctaaggcactaaacttcatcaatcctgatgaactttcaatgcaatgtatattgattgctttaaaccgatttcttcaggtgtgatcatttccttttcttaaacactatttcactagcattattgtgtgattgacagggtcattttatttccattagaatagttgaatggaatcagattagtaataaactttaagatggccttttatttcgcaaacatgttttgtggaacttacgatcaagttttttccctctttttctttaactaattgttatattatcaaattattctataggagaaacatggttctaagatggcctttttggatggcaatccacccgaaagactttgtatgccgatagttgatcatattcagtccttgggtggtgaagttcatctaaattcgcgcattcaaaaaattgagctaaatgatgatggaacggtgaagagcttcttactaaataatgggaaagtgatggaaggggatgcttatgtgtttgcaactccaggtgttttttcttcttcttattttacgaatgtaataattaattgttttcatgacattcttaatcttcactccttaacattttttaggttctttttcttgaaaacagtggatattctgaagcttcttctaccagataactggaaagggattccatatttccagagattggataaattagttggcgtcccagtcataaatgttcacatatggtaagtgatggcttttgatgttgaaaaccatatgcatgcatatgttaaactcctattttaaattaaattattacagtcatcattatttcattttgctgtctaatgtataaaaactcaaatattcactgaagagtttggaatagaaatctatgcaattcgtcttaaattgtattaatgacaattggcaatttataattaattttcttcttctaattgaatggttgtaatagttttggtaaatggaaactttatttgttaactttttttgtggtaaaataatgtcactatcactaattgatgtagtaatattctttgttattttttttttaaaagaaattcctctcactttattgatcttttttcaaaattttggataacgccaacaaagttaatttgtaatgtttgtggataatgattatattattatctccactggtgtgatgacatactatatatgcatcgcatttgcaggtttgacagaaaactgaagaacacatatgatcaccttctctttagcaggtcttcctcaaactttgaaacacctgtaatgtctggaagggatagaaattctgttcatttaatgcctgttttgtttttctgatacatagatgtatattgagtcaacttttgtgcttcataagttgataaaatttgacaagtataacacataaatgtttactaagggataagaacatttttcattataatattgcaaaataacaatcagaagccaaactgaatattgttatacgcaaatagtgtagaagatatcctctccctagacactctaatattgtgtcatttatttggttgggaaattgatactgacctatctattactaacatgtgataacaggagagaggatatctattgaggcttgtgacatgccctaatcttggaatgaagtattggtctcattctgctattacttaataaagaaagaaagtgataaagggaattaaaaatttgtggccaaggagatcctttctcccattgtttgcttaacttggttgagagtttgttcaagaatttattcagggtgttgatggggtttagcatgaggtggtttattgtgattgtgtatttggtagtgtttcatctacaatttccaattgctaaggataaaaggaatatttttactccgattgcaattgatacaatctggttatttgctagaggatttttaattgtgttgtgtatctccaagtgtctggggattttagggatgaacatcacttaagttttgctgtgtgaaatgaactttttttcttctttgctttcttgatggtatttgtggattataaataaagtattttcattacctcacatcttgtatattttatatttttcttcatgcagaagtccccttctgagtgtatatgctgacatgtcagtaacttgcaaggtagaaaccacttcatacatgttttgcactgatttatttcctcaacccatgcaaaagttgtggactgcatgtctaggttagcatattcctttatatatttatatataactagggtcacctaaatgagttggagatagcgctttctggataaacagtaaaccctctctacaaattaattcagttttcagacatttatataatactcttgttgtttgaggctgaatttcttctgcatatggtgatatcatcaaatgttggaatgttgtttgagtaactgtttcactgtaatgcaggaatattatagcccaaaccagtcaatgttagagttggtttttgcaccagccgaagaatggatttcacgtagtgacgatgatattattcaagccacgatgactgagcttgccaaactctttcctgatgaaatttctgcagaccaaagcaaagctaagattctcaagtaccatgttgttaaaacaccaaggtttgattgaatacccacagagggtggattatttcttttagcttcttttataaagatggatttggaatgtctttgttgttacttaactaatagtgacatttgtgttctgaatcctaatggaactgactacattgtttcaggtcggtttacaaaactgttccaaattgtgaaccttgtcgacccattcaaagatctcctatagaaggtttctatttagctggagattacacaaaacaaaaatatttagcttcaatggaaggcgctgttctttctgggaagctttgtgcacaggctattgtacaggtaaaatctgacacaaaaatatctatgtaactcgcagcaatacattagcaagaaatgtgataatctatatatacatgctttcttggtaattttgtgagagaaacttgattgtgaattcagcaggatgaataacgctacattgtgttgtggaaatcatacactgaagttctcagcttttattcgttgatttaatgaatctttccactttaattttttgattgaaacatttttattaagttccaatcatcctcaaaacttatgctgtctctgaatgattcttgtatgtatctccaatgcccccctcatgcaaaagaccttcggttatgaacattatttgaaattaatatgaaagaattagcttaatgaggattgatcttctgcataggttgatttgaatgtttttctaatcattggtgtcattgaggctttttaaaaattatgcaggattctgagctactagctactcggggccagaaaagaatggctaaagcaagtgttgtgtaacaaaaacaagaattgaaagagtcatggtagagtacaggagcatcatttcaactttggcattctttgtctgtggtcaggactcaggagaccttcaactttattagttcatacgaataaagaaaggctcagcttctgaaatttagctcaccgtcgtcaactgtgtgcaataagctatacggaacaaacgacatgtgtcaacttaaagtcagcccattgttttgttatcctccaattttctggatcaatgtttgtattggaaagaaatatgtcattattcaaacttgtttatatccactttttttatttatcaacatttgtcacaacctttcgttgagtagcaaattattcccagaaaggcacattacatatatatatatgcattgtatattacacacaatcatatatatatttatacaaaataa

SEQ ID NO: 22 - DNA, 20 bp, Artificial Sequence
  Nucleotide sequence of Gm_D7_PDS18 gRNA (target sequence)
ctggaagcaagagacgttct

SEQ ID NO: 23 - DNA, 7864 bp, Artificial Sequence
  Nucleotide sequence of T-DNA region of pNMD27570
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccactctgtgtgccttgatacacgcaatcgaaccggagagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaatgctccactgacgttccataaattcccctcggtatccaattagagtctcatattcactctcaatccaaataatctgcaccggatctggatcgtttcgcatgtctcctgaaagaaggcctgctgatattagaagggctactgaggctgatatgcctgctgtgtgcactattgtgaatcattacattgagacttctactgtgaatttcaggactgagcctcaagaaccacaagagtggactgatgatcttgtgaggcttagagagaggtatccttggcttgttgctgaagtggatggtgaagtggctggtattgcttatgctggtccttggaaggctaggaatgcttacgattggactgctgagtctactgtttacgtgtctcctaggcatcaaagaactggtcttggatctactctttacactcatcttttgaagtctttggaggctcagggtttcaagtctgtggttgctgtgattggacttcctaatgatccttctgtgaggatgcatgaggctcttggttatgctcctaggggaatgcttagagctgctggtttcaaacatggaaattggcatgatgtgggattctggcagcttgatttctctcttcctgttcctcctagacctgttcttcctgtgactgagatttgagcgggactctggatctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgcttgctgaattggagagccgccgcaagaattcaagcttggaggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagcttttcatccagctcgtgcagacctacaaccagcttttcgaagagaaccctatcaacgcttcaggtgtggatgctaaggctatcctctctgctaggctctctaagtctagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagggtgaacaccgagatcaccaaggctccactttctgcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatctccttaggaagcagaggaccttcgataacggatctatccctcatcagatccacctcggagagttgcacgctatccttagaaggcaagaggatttctacccattcctcaaggataacagggaaaagattgagaagatcctcaccttcaggatcccttactacgttggacctctcgctagaggaaactctaggttcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagcttcagaacgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggatcctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagttgcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagcttggaggtgatggatcacctaagaagaagaggaaggtttcatcaggtggtgctgctggatgagcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctactagaggatgcacatgtgaccgagggacacgaagtggtttaaactatcagtgtttgacaggatatattggcgggtaaacctaagagaaaagagcgtttattagaataatcggatatttaaaagggcgtgaaaaggtttatccgttcgtccatttgtatgtgccaacca

SEQ ID NO: 24 - DNA, 4155 bp, Artificial Sequence
  Coding sequence of Cas9 endonuclease gene from Streptococcus pyogenes codon-optimized for Arabidopsis
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagcttttcatccagctcgtgcagacctacaaccagcttttcgaagagaaccctatcaacgcttcaggtgtggatgctaaggctatcctctctgctaggctctctaagtctagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagggtgaacaccgagatcaccaaggctccactttctgcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatctccttaggaagcagaggaccttcgataacggatctatccctcatcagatccacctcggagagttgcacgctatccttagaaggcaagaggatttctacccattcctcaaggataacagggaaaagattgagaagatcctcaccttcaggatcccttactacgttggacctctcgctagaggaaactctaggttcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagcttcagaacgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggatcctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagttgcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagcttggaggtgatggatcacctaagaagaagaggaaggtttcatcaggtggtgctgctggatga

SEQ ID NO: 25 - DNA, 8282 bp, Artificial Sequence
  Nucleotide sequence of T-DNA region of pNMD34661
tgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaatgcctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagcctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggaggcgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatagcatccatggcctccgcgaccggctgcagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcattgtaattgtaaatagtaattgtaatgttgtttgttgtttgttgttgttggtaattgttgtaaaaatacagtatgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctgcaagaattcaagcttggaggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctactgtataagagctcatttttacaacaattaccaacaacaacaaacaacaaacaacattacaattacatttacaattatcgatacaatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagcttttcatccagctcgtgcagacctacaaccagcttttcgaagagaaccctatcaacgcttcaggtgtggatgctaaggctatcctctctgctaggctctctaagtctagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagggtgaacaccgagatcaccaaggctccactttctgcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatctccttaggaagcagaggaccttcgataacggatctatccctcatcagatccacctcggagagttgcacgctatccttagaaggcaagaggatttctacccattcctcaaggataacagggaaaagattgagaagatcctcaccttcaggatcccttactacgttggacctctcgctagaggaaactctaggttcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagcttcagaacgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggatcctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagttgcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagcttggaggtgatggatcacctaagaagaagaggaaggtttcatcaggtggtgctgctggatgagcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctactagaggatgcacatgtgaccgagggaatccgtttaaactatcagtgtttgacaggatatattggcgggtaaacctaagagaaaagagcgtttattagaataatcggatatttaaaagggcgtgaaaaggtttatccgttcgtccatttgtatgtgcca

SEQ ID NO: 26 - DNA, 7948 bp, Artificial Sequence
  Nucleotide sequence (genomic) of Nicotiana benthamiana phytoene desaturase gene NbPDS3a (Niben101Scf01283g02002.1)
  misc_feature (5066)..(5127): n is a, c, g, or t
  misc_feature (6274)..(6650): n is a, c, g, or t
gaatgagcaaagcaagaaattaaaaagagagagaggtgctttatccatcaaatgtggctatggtaggaagagccaatggtgggacatttttggagtgtagccaaaacataaaggaaggtccagtgcgagttactgcaaattgagttgggagtgaggattaaagaagatagtaacatatttctagctaaatagcaaacaaatgatccgttaacagaagtggccaaaccaccaaattcaggcatctccaccaaatattagttttttatacacaaaagattcaacacaaacagttaagtacttctttaatcgttcctaattctttgttcaggggtatctttttgtgggtaacggccaaaccaccacaaattttcagttcccactcttaactctttcaacttcaacacaacaaattagtatttgcttttccttctttgcttatctagtgcataacgattttctacaactttagcatagtccacaacgtgaaacacaactccttggcggtttataccgaggtaagaaatgattttggtttctttggttacatcagctgaatgctttgcttgagaaaagctctctttttcccgtttaggatcttgtttatttgctttcgtttttctactcgtttgaattttaacttgattttgtgggtgaaggctaatttttctcatagtgtaagaacaagtttcatatgtactgtaaaagctagaatcttttttacttttgcatataaatttgtgtaataaatgcttaagaaccagaatatttgaaaaagataaggaattttgcatagtatttaggttcacaagtgggacaatcttcttacactgaaatatctttatgtcaggcttaatttactgctatcttgttcaataaaatgccccaaattggacttgtttctgccgttaatttgagagtccaaggtaattcagcttatctttggagctcgaggtcttcgttgggaactgaaagtcaagatgtttgcttgcaaaggaatttgttatgttttggtagtagcgactccatggggcataagttaaggattcgtactccaagtgccacgacccgaagattgacaaaggactttaatcctttaaaggtttgttttgaatgcgaaagtgtgatgctggatttatgatcgtgggcatatatcctctaaaataagagatgtatatcttgccattcaggtagtctgcattgattatccaagaccagagctagacaatacagttaactatttggaggcggcgttattatcatcatcgtttcgtacttcctcacgcccaactaaaccattggagattgttattgctggtgcaggtgattttttccagccatctatatttgtagttttcatttttctttctttggaaggaagatcattctattagttatattatcactagaatatttacctgtacattcttttctgattaactgttttggaccgcaaaattttaggttcttacttcttcgccattttgcaactaatcagcaattaggagcggtttgaaaactagtttgttttgaactatttttgccgtcactctatttatatactgttgaattgtcccaaatcggtggaatttgaggtccttggtctcatctcataagctagcttttggggttgagttaccacatcggtgggatttgaggtccttcgtctccttatatgttcttggacaagcttcacctcataagctagcttttggggttagagttaggcccaaggtccatttatcatatgcttgtctattctctcttatcatctgagccatgataagcgggtgaacgtgctgtctattgggtggcatgtccaaaggatcattctgaaatattggaggcaaatgaaccaataccttgtgcaagattgatctcactatacctataatcagagtactgagttccaaaaatttcaaaacccattgaaaagtcaaacgagttacatataggggttgcactcttctacggcttgcaatatgtgagaaaaagatgagaagtcgatcttcatatttcatctttactaggctggaccattgactggttagcagttttgaacttgttcttcaacttggcttgcatggtactgtgccgatcatttcttttgtattgtcatcagctggttgattatctgagtacctaaagaaagaatgttatatgcatgatatattctactgtactataaaagatataataaagaatgctagccgaggtactgcatggccttttcagataaatagaagctgtagcatgattctaattcaatttttttgggaatatcaggtttgggtggtttgtctacagcaaaatatctggcagatgctggtcacaaaccgatattgctggaggcaagagatgtcctaggtgggaaggtgaagaatatccaatctttcctttaattttattcctttttcttttgtgtccttccctattgatagtcccttttcaggaaggcttctgtttgttttatttgaaatcatttttcatactctttaagcattcagttgctcaaacaattgcaaggatattcactattcctaattttgaccgtcttcttttctctcagtttagttttattcccctctctttttgaaggaaatagatctgtcctaaaaatttccagctttactactaatagtgttaattgtcgataaaatagtacatcatattaggtaaaagatatggactgtatattattatcattctctattattttaaactgagtcaattttaaccgtcctgttgggtgcatttctcatataaacagtcttttctgtgagatgctatgtgaattagctgattgttttggtatagagcactatgttagtcagttttatcttactgaagcagtcaccaagagtctagttgtataggctagaagattgaattagcattaatctttatgtgttctgcacctgaatacttgtacctcccttttaggtagctgcatggaaagatgatgatggagattggtacgagactgggttgcacatattctgtaagtttgactcctcaagaatgctactttaatcttctaatacagtcatagcaatttctttcaagatctcttttattaatcagatagctatccctgtttgtcttttgtcttttgcaaatagccaatttttgtcagtcgatctgtattctgccttgcctctctttatttatctgctaactcgtatggtgactcatacaagttggtgcatctcctttaagttggggcttacccaaatatgcagaacctgtttggagaactagggattgatgatcggttgcagtggaaggaacattcaatgatatttgcgatgcctaacaagccaggggagttcagccgctttgattttcctgaagctcttcctgcgccattaaatggtaagtacttaatcatgagtaaatttctcccttcagcgttgattatgcaaacttccccaataaggtatgaaattgattagtcttaataccctggcacattgctaacatcaaaagaacataaaggttcattacgtcttgatcagaatttctgcatgtagctaaagtgaatgagtgtctgtatagatttttacacattgcaagcataagcctgttatgttatctctttttttcatttctctacctgtatctcttattctcatttctctatctatgcgttattacttctacaggaattttggccatactaaagaacaacgaaatgcttacgtggcccgagaaagtcaaatttgctattggactcttgccagcaatgcttggagggcaatcttatgttgaagctcaagacggtttaagtgttaaggactggatgagaaagcaagtgcgtgatcgttttatcttattctttaaagttcataaccttgaggacatagttgacttgcatattgttgatttaacatgttcgaattgtctacctgcctttctttttctaacaacatagatcttacaatctcagcagcagctatttgcttaatgcttttcagggtgtgcctgatagggtgacagatgaggtgttcattgccatgtcaaaggcacttaacttcataaaccctgacgagctttcgatgcagtgcattttgattgctttgaacagatttcttcaggttagaatcctgatccaccctcaaaacaaaaagagagaaagggatataatcctaccaaagctgtaaatcatgttagggacctgacatatcggtgcaggaaacttatgagtgaacttgtccactctgtttaacttttctgatatatttgaattattaatctgcaggagaaacatggttcaaaaatggcctttttagatggtaaccctcctgagagactttgcatgccgattgtggaacatattgagtcaaaaggtggccaagtcagactaaactcacgaataaaaaagatcgagctgaatgaggatggaagtgtcaaatgttttatactgaataatggcagtacaattaaaggagatgcttttgtgtttgccactccaggtataatatccattatactagtatgacgcttccagttttcacattttaatatgaatttatagttttttgctgacttttgattatccaattagtggatatcttgaagcttcttttgcctgaagactggaaagagatcccatatttccaaaagttggagaagctagtgggagttcctgtgataaatgtccatatatggttagtgatgaaaattttgcttttcagtgtttggtcttcctctagcatatctatgtatgtgcatgttaatgtctatacgtacatgtttatgtggtcctcccgtattgtgtttacttcccttgaatgaggaacttatggatgtacgcttttccaactttgattgtacacattgcaattgtctgttcaactttgaggagcagaacttccattgtttagctattagtggctgagattcctgctgaaaagatttgtataaatttaatttgcaggtttgacagaaaactgaagaacacatctgataatctgctcttcagcaggttcatttttgatcaattttattgttccagaccagtttctgcgtgtccatgactacattctcatattagctccccccccccnnnnnnnnnnnnnnnnnnnnnnnnnnnnnnnnnnnnnnnnnnnnnnnnnnnnnnnnnnnnnnccccccccccccggtctcttttttgccatttaaatgagaccttacaatttgtttagtactctaccatagttttttaatcaataagccaaaggggaaaaactaataaaagtgtataaaatttcttcctgtattagtccaattctttcgcaacttatattgttaattattatttatcttttggattgaaatggattttgtatatctaataatataaacaaatatatctcttcctcttataagatttttcaccatagaaaaatgctcccataaggtcagtcattctggctaaatatcccacacttcaaccattgagatattttgttctttgcatccaggaatacatttggcatcaatagataggaatcaatgaagatatattatcaatttcctgcaagtttcttggcactagaaacattagatccatatcatgtaaattgcctttgttaaattgaaggtctatgaaatttgggttggtttgaaaaccttttgtttttcccccccacatccctaatcgtttatttagtcaaggtcagacctgacatgttatgatgaccatttctccaaggcatttataatggactggagtatccatgccacatttcatcagctacatgtcgattatgttcccctacttttaaatggcaccattgttggtggagcaagattatagattttcctgatacttgtatgggttcccttgctcaatctctcttttacttcatgcagaagcccgttgctcagtgtgtacgctgacatgtctgttacatgtaaggtattgactcgtctgtaccattatactggtctaatctgttgggtatgagttgctggtaaattgcataatgcttgttggatttgtgtgtgagttgctgctagatctatgtcctgctatatttatgtatgagttgctgttgttgcaatcttcatttcgaatgcataatgatataggttctgtatgtacggaatagtcaggacaatgctcctgtctgtgcacgggggctctacaggaagcaactttcggaggagaagtacagaaagtgtgatgaatcttaaggcggttaaagtagtttcttttagctaaattttgaaataatttgaaggaggggaaaacgctctctcagtctgtggttgcattggttgtggggggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggagggaccttaattcagtgttacctgcatataaatcagactaaagcctggagatcagacgttctgcatataaatagataattaataatgatctcgtaatactctaaagcctggagatcagactgttttaactatcctgagatgattacttttactctcggattagcttaggcgagctgcaagactacatcgaatctttagaaatgggaacataaaaaaggtgcgaagtggggaagtggctgaacaataggcatatgtgagtgagtggggagtaaaattacttcctttacttgggtacagtcaagaatggatgacagcttagcccactatatctgttcatgtgttctttagggtcctctgatataactggtctctctgcaggaatattacaaccccaatcagtctatgttggaattggtatttgcacccgcagaagagtggataaatcgtagtgactcagaaattattgatgctacaatgaaggaactagcgaagcttttccctgatgaaatttcggcagatcagagcaaagcaaaaatattgaagtatcatgttgtcaaaaccccaaggtcagtaatcattttgctttcatagttgtgtagtatgcgagaattactgtccacgtggaatctattcctgttatgaatcctgattaatctgctttttactttcaggtctgtttataaaactgtgccaggttgtgaaccctgtcggcccttgcaaagatcccctatagagggtttttatttagctggtgactacacgaaacagaagtacttggcttcaatggaaggtgctgtcttatcaggaaagctttgtgccgaagctattgtacaggttagctctcacatttttttcccttccattgatagtgtatttgattatattttgtcatctttgctgcggtagagaattttagaagcatttctcagacattagttagcagagttactcaggatatctgcagttttggagcttcagtagtagcatgataaaatgcagaggattgtgttttttcattctttattaaaccttgtgccaaaggtcttttggaaacaacctctctaccccgaggtaggggtaaggtctgcgtacatattaccctccccataccccatgcgtgggattatactgggtggttgttgtataaacctatatctctataatttgcaggattacgagttacttcttggccggagccagaagatgttggcagaagcaagcgtagttagcatagtgaactaaaatgttaattctgtacacaaaatttaagatgaaggcggccacgctgaattag

SEQ ID NO: 27 - DNA, 8895 bp, Artificial Sequence
  Nucleotide sequence (genomic) of Nicotiana benthamiana phytoene desaturase gene NbPDS3b (Niben101Scf14708g00023.1)
  misc_feature (2763)..(3070): n is a, c, g, or t
  misc_feature (4364)..(4364): n is a, c, g, or t
  misc_feature (5293)..(5293): n is a, c, g, or t
  misc_feature (5675)..(6605): n is a, c, g, or t
cctcaatgacccagtaacccaagtgggagatgtgtgcaaagtggtcaaatcttagaaggaatgagcaaagcaagaaattaaaaagagagacagagaggtgttatccatcaaatgtggctatggtcggaatagccaatggtgggacagtctagccaaacataaaggccggtccagtgcgagttgctgcaaattgagttggagtaaaaaattaagataccatatttccagctaaatagcaaacaaatgacccaccattaacggaagtggccaaaccaccaaattcaggcatctccaccaaaaattagttttttatacacgaaagattcaacaattagtatttctttaagccttcctaattctttgtcaggggtatctttttgtgggtaacagccaaaccaccacaaattttcagttcccactcttaactctttttaacttcaacacaacaaattttttgcttttccttctttgtttatcttgtgcataacgatttcctacaactttagcataatcttggtttgtaatccacaacgtgaaacacatcacctaggcggtttcataccgaggtaacaaatgattttggtttctttggttacatcagctgaatgctttacttgagaaaagctttctccttttcccgtttaggatcttgtttatttgctttcgtttttctactcgttaaaattttaacttgattttgtgggtgaattataactttactcatagtgcgagaacaagtttcgtatggactgtaaaagctagaatcttttttacttttgcatataaatttgtgtaataaatgcttaagaaccagaatattgaaaaaacaaaggaattctacatagtatttaggttcacaagtgggacaatcttcttacagtgaaatatctttatgtcaggcttaatttactgctattttgttcagtaaaatgccccaaattggacttgtttctgccgttaatttgagagtccaaggtaattcagcttatctttggagctcgaggtcttctttgggaactgaaagtcaagatggtcgcttgcaaaggaatttgttatgttttggtagtagcgactccatggggcataagtttagaattcgtactcccagtgccatgaccagaagattgacaaaggacttcaatcctttaaaggtttgttttgaatgcgaaagtgtgatgctgaatttatgatcacgagcatatattctctaaaataagatatcttgccattcaggtagtctgcattgattatccaagaccggagctagacaatacagttaactatttggaggcggcgttatcatcatcatcatttcgtacttcctcacgcccaacaaaaccattggagattgttattgctggtgcaggtgattttttccagtcatctatatttgtagtcttcatttttctttctttggaaggaagatcattctattagttgtattatcactagaacatttattgtgcattcttttcttattaactgttttggaccgcaaaattttaagttcttacttcttcgcctcccaactgattagattaggagtgatttgaaaattagtttgttttgagctatttttgccgtcactcatatactgttgagttgtcccacatcggtgagatttgaagtccttggtctcacctcataagttagcttttggggttgagttaggcccaatatccatttatcatagtacgagagccaggcccatcccagttattgttaccaatgtcgggctcctatttatgttgtccacgctccagtttgcaagcctaggcgtgggggaggggggtgttgagttgtcccacatccgtgggatttgaggtacttggtctccttatatggtcttggacaatccataagctagcttttggggttgagttcggcccaatgttcatttatcatatatatatatatatatatatatgtctattctctcttaccatctgagccatgataagcgggtgaacgtgctgtctattggatggcatgtccgatggatcattccgaaatattggaggcagatgaaccaataccttgtgcaagattgatatcactatacctataatcagagtacttagagttccaaaaatttgcagaacccattgaaaagtcaaacaagttacatataggggttgcactcttctaaggcttgcaatctgtgagaaaaagatgagaaggagatcttcatatttcatctttattaggctggaccattgaccggttagcagttttgaacttgttcttcaacttggcttgcatagtactgtgccgatcatttcttttgtattgtcatcaactggttgattatttgagtacctaaagaaagaatgttatgcatgatacattgtgctgtactataaaagatataataaagaatgctagccgaggtactacatggccttttcagacaaatagaagctgtagcatgattctaattcgatttgttttgaaatatcaggtttgggtggcttgtctacagcaaaatatctggcagatgctggtcacaaaccgatattgctggaggcaagagatgtcctaggtggaaaggtgaagaatatccaatctttcctttaattttattcctttttcttctgtgtccttgcctattggtagtccctgttcaggaaggcttctgtttgttttatttaaaatcatttttcatactctttaaacattcagttgctcaaacaattgcaagggtgttcactattcctatttttgactgtcttactttctctcagtttagttttattcccctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccccccccctgtctctctctctctctctgtttttggaggaaatagatctgtcctaaaaacttccagctttactactaatagtgttaattgtcgagaaaatattacagcatattaggtatatggaaagtatattattattctctattattttaagattgagtcaattttacccgtcctgttggttgcatttctcacataaacagtcttttctgtgagatgctatgttaattagctgatgtttttggtatagagcactatgttagttttatctttactgaagcagtcaccaagaatctagttgtataggctaaaagattgaattagcattaatctttatgtgttttgcacctgaatacctatacctaccttttaggtagctgcatggaaagatgatgatggagattggtacgagactgggttgcacatattctgtaagtttgactcctcaagaatgcatactttaatcttctaatacaacagtttctttcaagatctcttttctctattaatcagatagatatcgctgtttgtgttttgtcttttgcaaatagccaatttttgtcagtcgatctgtattctgccttgcctatctttttttatctgttaatttcatatggtgactcatacaagttggtgcatcccctttaagttggggcttacccaaatatgcagaacctgtttggagaactagggattaacgatcggttgcagtggaaggaacattcgatgatatttgcgatgcctaacaagccgggggagttcagccgctttgattttcctgaagctcttcctgcgccattaaatggttagtacttaatcatgatttctcccttctgcattgattatccaataaggtatgaaattgattagtccattgaccattaatactctggcacattgctaacatcaaaagaacataaaggttcattatgtcttgatcagaatttctgcatgtagctaaagtgattgagtgtctgtgtatatttttatacattgcaagcataagccagttatgttatctcttattttcatttctctatcgatgcgttattacttctacaggaattttagccatactaaagaacaacgaaatgcttacatggcccgaaaaaatcaaatttgctattggactcttgccagcaatgcttggagggcaatcttatgttgaagctcaagacggtttaagtgttaaggactggatgagaaagcaagtatgtgatcgttttatcttattctttaaagttcataaccttgaggacatagttgacttgcatgttgttgatttaacatgttnatgtgatcgttttatcttactctttaaagttcataaccttgaggacatagttgacttgcatgttgttgatttaacatgttagaattgtctacctgcctttctttttttaacaacatacatcttacaaatctcagcagcagctatttgcttaattgcttttcagggtgtgcctgatagggtgacagatgaggtgttcattgccatgtcaaaggcacttaacttcataaaccctgacgagctttcgatgcagtgcattttgattgctttgaacagatttcttcaggttagaatcctgatccaccctcaaaacaaaaagagagaaagggatataatccgaccaagctgtaaatcatgttagggacctgacatattggtgcaggaaacttatttgtgaacttttccactctgtttaacttttctgatatatttgaattattaatctgcaggagaaacatggttcaaaaatggcctttttagatggtaatcctcctgagagactttgcatgccaattgttgaacatattgagtcaaaaggtggccaagtcagactaaactcacgaataaaaaagattgagctgaatgaggatggaagtgtcaaatgttttatactgaataatggcagtacaattaaaggagatgcttttgtgtttgccactccaggtataatatccattatactagtatcgatgcttccagttttcacatttttaatatgaatgtataattttttgctgacttttcattatccgattagtggatatcttcaagcttcttttgcctgaagactggaaagagatcccatatttccaaaagttggagaagctagtgggagttcctgtgataaatgtccatatatggttagtgatgaaaattttgcttttcagtgtttggtcttcctctagcatatctatgtatgtgcatgttaatgnacttttcagtgtttgttcttcctctagcatatctatgtatgtgcctgttaatgtctatacatacatgtttatgtggtcctccggtattgtgttaacttcccttgagcgaggaacttatggatctacgcttttccaaaactttgattgcacacattgcaattgtctgttcaactttgatgagcagaactaccattgtttagctattagtggctgagattcctgctgaaaagatttgtataaatttaattgcaggtttgacagaaaactgaagaacacatctgataatctgctcttcagcaggttcatttttgatcaattttattgttccagagcagtttctgcgtgtccatgactacattctcatattagctccccccccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacatgttatgatgaccatttctccaaggcattttatccatgccacgtttcatcagctacatgttgactatgttcccctactttttaaatggcaccattgttggtggagaaagattatagatgttcctgatacttgtatgggttcccttgctcaatctctcttttacttcatgcagaagcccattgctcagtgtgtatgctgacatgtctgttacatgtaaggtattgactcgtctgtaccattcatactggtctaatctgttggatatgagttgctggtaaattgcataatgcttgttggatttgtgtgtgagttgctgctagatctgtgtcctgctatatttatgtatgagttgctgctattgtaatcttcatttaggatgcttaatgatataggttctgtatgtatggaatagtcaggacaatgctcctgtctgtgcacaggggctctacaggaagcaactttcgaaggagaagtaaagaaagtgtgatgaacagggaaagtagtttcctttagctaccttaattcagtgttacctgcaatgttcagtgtttggagagaggcgataagcctacttcttaattttgttagaaaatgcgtacaaaatataaatcagtagttactaaaaagttggagaagtagtgggatcttttcgctatttttaacccagaataagacagctatgccatatagctttgattatccgttaacgttctgtatataaatagataattcataataatgtcgtaatactaaagcctggagatcagactgctttaactatcctgagatgattacttttactcttggattagcttaggcgagccacaagactacattgaatctttagaaatgagaacataaaaagggtgcagaagtggggaagtggctgaacgatatgcatatgggagtgagtggggagtaaaattatttcctttacttgggtacaatcaagaatgaatgacaacttagcccactatatccgttcatgtgttctttagggccctctgatataattggtctctctgcaggaatattacaaccccaatcagtctatgttggaattggtatttgcacctgcagaagagtggataaatcgtagtgactcagaaattattgatgctacaatgaaggaactagcaaagcttttccctgacgaaatttcggcagatcagagcaaagcaaaaatattgaagtatcatgttgtcaaaactccaaggccagtaatcatttgctttcatacttgtgcaatatacgagaactgcagtccacgtggaatctattcctattctgaatcctgattaatctgctttttttctctcaggtctgtttataaaactgtgccaggttgtgaaccctgtcggcccttgcaaagatctcctattgaggggttttatttagctggcgactacacaaaacagaaatacttggcttcaatggaaggtgctgtcttatcaggaaagctttgtgcccaagctattgtacaggttagttctcacagttgtttttgtccactaatagtatatttgatcaaattttgtcatctttgctgcggtagagaatttttgaagcatggacgtcaagcatgcctcttacttataattgctaatttgcggaatagttctccaaccaatatagtgttaaaccaaaaaaataaaattgtgcacacagatcacagagttgctcaggatatctgcatttttggagcctcagtagtagcatgataaaatgcagaaggttatgtttttttcattctttattaaatttatatctctatattttgcaggattacgagttacttcttggccggagccagaagaagttggcagaagcaagcgtagtttagcatggtgaactaaaatgttgcttctctacactaaatttaagatgaaggtggccacactgaattagcgttgtagacaacacatacaaggacagtacaacatttaacccaaatacgagaaatgttacacaaatatgtgctctgctttccctccgatttagttcgcaagttactaattataagatggaattgaatgaaaccaaagacggataaagaccctaaactataagataagtaagcctctccagaccatacaagtgcgcgtcgagcccatgcgaagggtttggttaagatatgccagattccaccaagtatacaaatgaaacctaaccatacatgtcctccgattatataatgcaaattgattcattcaaactaaacttttaagcgtcacagttatactagcaaatacctttaagacattaagcttcacgtcttaaaacatca

SEQ ID NO: 28 - DNA, 59 bp, Artificial Sequence
  Reference sequence 1 (fragment of Nicotiana benthamiana phytoene desaturase gene NbPDS3a (Niben101Scf01283g02002.1; SEQ ID NO 26), nucleotide position 1201-1259)
attatccaagaccagagctagacaatacagttaactatttggaggcggcgttattatca

SEQ ID NO: 29 - DNA, 79 bp, Artificial Sequence
  Reference sequence 2 (fragment of Nicotiana benthamiana phytoene desaturase gene NbPDS3a (Niben101Scf01283g02002.1; SEQ ID NO 26), nucleotide position 1214-1292)
agagctagacaatacagttaactatttggaggcggcgttattatcatcatcgtttcgtacttcctcacgcccaactaaa

SEQ ID NO: 30 - DNA, 79 bp, Artificial Sequence
  Reference sequence 3 (fragment of Nicotiana benthamiana phytoene desaturase gene NbPDS3b (Niben101Scf14708g00023.1; SEQ ID NO 26), nucleotide position 1260-1338)
ggagctagacaatacagttaactatttggaggcggcgttatcatcatcatcatttcgtacttcctcacgcccaacaaaa

SEQ ID NO: 31 - DNA, 85 bp, Artificial Sequence
  Reference sequence 4 (fragment of soybean phytoene desaturase gene GmPDS11 (Glyma.11G253000; SEQ ID NO 20), nucleotide position 1334-1418)
tgatgctgggcataaacctatattgctggaagcaagagacgttctaggtggaaaggttttcctgctaatttaatctcttacgtca

SEQ ID NO: 32 - DNA, 85 bp, Artificial Sequence
  Reference sequence 5 (fragment of soybean phytoene desaturase gene GmPDS18 (Glyma.18G003900; SEQ ID NO 21), nucleotide position 1173-1257)
tgatgctgggcataaacctatattgctggaagcaagagacgttctaggtggaaaggttttcctgctaatttaatccctttcggca

SEQ ID NO: 33 - DNA, 26 bp, Artificial Sequence
  Primer PDS_in2_fwd
gtgtgatgctggatttatgatcgtgg

SEQ ID NO: 34 - DNA, 29 bp, Artificial Sequence
  Primer PDS_in2_rev
ctagcttatgagatgagaccaaggacctc

SEQ ID NO: 35 - DNA, 69 bp, Artificial Sequence
  Primer PDS_ex3_wob_fwd
  misc_feature (34)..(43): n is a, c, g, or t
tcgtcggcagcgtcagatgtgtataagagacagnnnnnnnnnngtagtctgcattgattatccaagacc

SEQ ID NO: 36 - DNA, 72 bp, Artificial Sequence
  Primer PDS_ex3_wob_rev
  misc_feature (35)..(44): n is a, c, g, or t
gtctcgtgggctcggagatgtgtataagagacagnnnnnnnnnnctgcaccagcaataacaatctccaatgg

SEQ ID NO: 37 - DNA, 23 bp, Artificial Sequence
  Primer D7-PDS18_11_PCR_f
aaggtatcctgatatcatgttgg

SEQ ID NO: 38 - DNA, 23 bp, Artificial Sequence
  Primer D7-PDS18_11_PCR_r
aatcttcaacaagcctgatgatg

SEQ ID NO: 39 - DNA, 22 bp, Artificial Sequence
  Primer D7-PDS18_18_PCR_f
ttatggaattttaggtaccctg

SEQ ID NO: 40 - DNA, 22 bp, Artificial Sequence
  Primer D7-PDS18_18_PCR_r
tgatgaagttataaagccaacg

SEQ ID NO: 41 - DNA, 24 bp, Artificial Sequence
  Primer  trsv-sil-fwd
tcaatgctaaggacatagttgcac

SEQ ID NO: 42 - DNA, 25 bp, Artificial Sequence
  Primer trsv-sil-rev
tattgacgcttctatctaaccaacc

(19) United States
(12) Patent Application Publication
(10) Pub. No.: US 2022/0090107 A1
(43) Pub. Date: Mar. 24, 2022

(54) RNA VIRAL RNA MOLECULE FOR GENE EDITING

(71) Applicant: Nomad Bioscience GmbH, München (DE)

(72) Inventors: Heike Prochaska, Halle (Saale) (DE); Stefano Torti, Halle(Saale) (DE); Anka Thümmler, Halle(Saale) (DE); Patrick Romer, Stadt Südliches Anhalt OT Riesdorf (DE); Anatoli Gititch, Halle(Saale) (DE); Yuri Gleba, Berlin (DE)

(21) Appl. No.: 17/612,687

(22) PCT Filed: May 22, 2020
(86) PCT No.: PCT/EP2020/064327

§ 371 Date: Nov. 19, 2021

(30) Foreign Application Priority Data
May 23, 2019 (EP) 19176284.8

(51) Int. Cl. C12N 15/82; C12N 15/11; C12N 9/22

(52) U.S. Cl. CPC C12N 15/8213; C12N 15/11; C12N 2800/80; C12N 15/8203; C12N 2310/20; C12N 9/22

(57) ABSTRACT

The invention provides a plus-sense single-stranded RNA viral RNA molecule, comprising a segment encoding a movement protein, a segment encoding a coat protein and a segment that comprises a guide RNA (gRNA), wherein said RNA molecule can be translated, in infected cells, to a polyprotein comprising the movement protein and the coat protein.

### FIELD OF THE INVENTION

The invention relates to a plus-sense single-stranded RNA viral RNA molecule that comprises a guide RNA (gRNA) useful for gene editing in a plant or in plant cells. The RNA molecule may be a picornaviral RNA molecule. The invention also provides a DNA molecule, DNA construct or vector encoding the RNA molecule, and an Agrobacterium cell comprising the DNA molecule, DNA construct or vector. The invention further provides a plant, a plant tissue such as callus or shoot, a plant seed, or a plant cell containing the RNA molecule, or containing the DNA molecule, DNA construct or vector. The invention further provides a process of sequence-specifically affecting a target nucleic acid such as target DNA and of conducting gene editing in a plant or a plant cell. Further provided is a process of infecting a plant such as a crop plant (such as soybean) with a genetically-modified picornavirus.

### BACKGROUND OF THE INVENTION

CRISPR-Cas gene editing methods have experienced an enormous advance and wide application in recent years in numerous organisms. In these methods, a nuclease such as Cas9 is guided by a guide RNA (gRNA) to a site of a target nucleic acid, where it binds. The complementarity between the gRNA and the target nucleic acid determines the site where the nuclease introduces a double-strand break (DSBs) into the target nucleic acid. Some recent reviews on CRISPR-Cas gene editing methods are as follows: Wang et al., Ann.u. Rev. Biochem. 85, 216, 227-264; Lino et al., Drug Delivery 25, 2018, 1234-1257; Adli, Nature Communications, 9, 2018, 911; Ishino et al., Journal of Bacteriology 200(7) 2018, e00580-17; Thurtle-Schmidt et al., Biochemistry and Molecular Biology Education 46(2), 2018, 195-2015. Thus, methods of application of CRISPR-Cas systems for gene editing are widely known to the skilled person. Although there are several classes and types of CRISPR-Cas systems, the class 2, Type II CRISPR-Cas9 systems have mostly been used for gene editing for ease of application, since only a single multidomain effector protein is required to mediate cleavage of target DNA. Similar qualities has the class 2, Type V system making use of the Cpf1 nuclease; this system does not require a transactivating (tracr) RNA (WO2016205711; WO2017141173).

Regarding the delivery of the components of a CRISPR-Cas system, that are necessary for cleaving or editing a target nucleic acid, into cells, there are three general approaches (Lino et al., ibid). Provision of (1) DNA plasmid encoding both Cas9 (or another nuclease such as Cpf1) and the guide RNA, (2) mRNA for translation of Cas9 (or another nuclease such as Cpf1) along with a separate guide RNA, and (3) Cas9 protein (or another nuclease such as Cpf1) with guide RNA (ribonucleoprotein complex). As further summarized by Lino et al., vehicles used to deliver the gene editing system cargo can be classified into three general groups: physical delivery (such as microinjection and electroporation), viral vectors, and non-viral vectors. “Viral delivery vectors include specifically engineered adeno-associated virus (AAV), and full-sized adenovirus and lentivirus vehicles. Especially for in vivo work, viral vectors have found favor and are the most common CRISPR/Cas9 delivery vectors. Non-viral vector delivery is not as prominent as viral-based delivery; however, non-viral vectors possess several advantages over viral vectors and are a bourgeoning area of research. Non-viral vector systems include systems such as lipid nanoparticles, cell-penetrating peptides (CPPs), DNA ‘nanoclews’, and gold nanoparticles. There are additionally many delivery technologies that have not been demonstrated in the literature as suitable to CRISPR/Cas9 delivery, though they appear to naturally lend themselves to the application. Four such technologies are streptolysin O, multifunctional envelope-type nanodevices (MENDs), lipid-coated mesoporous silica particles, and other inorganic nanoparticles” (Lino et al., ibid).

Gene editing using CRRISP-Cas systems have also been applied to plants and plant cells, cf. Jaganathan et al., Frontiers in Plant Sciences 9, 2018, Article 985. T-DNA based delivery systems may be used to introduce the nuclease and the gRNA into plant cells. In spite of the progress that has been made in the delivery of CRISPR-Cas systems into target cells, effective delivery still remains an obstacle. Notably, for use of gene editing in plants and plant cells, an effective delivery method applicable to different plant species is still desired.

Therefore, it is an object of the invention to provide a process of conducting gene editing in a plant or a plant cell and components and genetic elements and tools therefor. It is another object of the invention to provide nucleic acid molecules suitable for gene editing in plants or for plant cells, notably for gene editing in soy bean.

### SUMMARY OF THE INVENTION

For solving this object, the present invention provides:
(1) A plus-sense single-stranded RNA viral RNA molecule, comprising a segment encoding a movement protein (also referred to herein as MP segment), a segment encoding a coat protein (also referred to herein as CP segment), and a segment that comprises a guide RNA (gRNA), wherein said RNA molecule can be translated, in infected cells, to a polyprotein comprising the movement protein and the coat protein; or a complementary RNA thereof.
(2) An RNA molecule comprising
   (i) optionally a segment encoding a picornaviral P2A protein,
   (ii) a segment encoding a picornaviral movement protein,
   (iii) a segment encoding a picornaviral coat protein, and
   (iv) a segment comprising a guide RNA (gRNA), preferably a single guide RNA (sgRNA); or a complementary RNA thereof.
(3) The RNA molecule of item 2, wherein said RNA can be translated, in infected cells, to a polyprotein comprising the proteins of items (i) to (iii).
(4) The RNA molecule according to any one of items 1 to 3, that is capable of replicating in a prokaryotic or eukaryotic cell to form replicated RNA comprising a poly-A tail at its 3′-end.
(5) The RNA molecule according to any one of items 1 to 4, comprising, preferably in this order in 5′ to 3′-direction,
   (i) optionally a segment encoding a TRSV (tobacco ring spot virus) P2A protein,
   (ii) a segment encoding a TRSV movement protein,
   (iii) a segment encoding a TRSV coat protein, and
   (iv) a segment comprising a guide RNA (gRNA), preferably a single guide RNA (sgRNA); or a complementary RNA thereof.
(6) The RNA molecule according to any one of items 1 to 5, wherein said gRNA comprises a guide sequence linked to a direct repeat sequence.
(7) The RNA molecule according to any one of items 1 to 6, wherein said gRNA is capable of forming a complex with a CRISPR effector protein, such as a CRISPR nuclease capable of cleaving double-stranded DNA (e.g. Cas9), optionally in the presence of a transactivating RNA.
(8) The RNA molecule according to item 6 or 7, wherein said gRNA is a single guide RNA (sgRNA) comprising a transactivating RNA in one RNA molecule.
(9) The RNA molecule according to any one of items 1 to 6 or 7, wherein said gRNA is capable of forming a complex with Cpf1.
(10) The RNA molecule according to any one of items 1 to 9, wherein the RNA molecule further encodes a protein of interest to be expressed in a plant host.
(11) The RNA molecule according to item 10, wherein the protein of interest is a CRISPR effector protein (such as a CRISPR nuclease) capable of forming a complex with the gRNA.
(12) The RNA molecule of any one of items 1 to 11, which is a nepoviral RNA, preferably the RNA2 of a segmented nepoviral RNA.
(13) The RNA molecule of any one of items 1 to 12, which is an RNA2 of a tobacco ringspot virus (TRSV).
(14) The RNA molecule of any one of items 1 to 13, wherein said gRNA is present in the 3′-untranslated region (3′-UTR) of said RNA molecule.
(15) DNA molecule, DNA construct or vector encoding the RNA molecule of any one of items 1 to 14.
(16) Agrobacterium cell containing the DNA molecule according to item 15.
(17) The Agrobacterium cell according to item 16, containing a plasmid comprising in T-DNA the construct of item 15.
(18) A process of sequence-specifically affecting a target DNA, such as gene editing, in a plant or a plant cell, comprising the following steps:
   (i) transfecting said plant with the RNA molecule of any one of items 1 to 14 or with the DNA molecule of item 15, and
   (ii) transfecting said plant with a vector encoding proteins necessary for replicating said RNA molecule,

wherein said gRNA is capable of hybridizing with said target DNA and wherein steps (i) and (ii) can be performed in any order or simultaneously.
(19) The process according to item 18, further comprising providing said plant or said plant cell with an effector protein capable of forming a complex with the gRNA of said RNA molecule and with a target nucleic acid in said plant or plant cell.
(20) The process according to item 19, wherein said RNA molecule encodes said effector protein and comprises said gRNA.
(21) The process according to item 18 or 19, wherein said plant or said plant cell is transgenic for a gene encoding an effector protein capable of forming a complex with the gRNA of said RNA molecule and with a target nucleic acid in said plant or plant cell.
(22) The process according to any one of items 19 to 21, wherein the effector protein is Cas9 or Cpf1 or a variant thereof having a nuclease activity removed by genetic engineering such as by mutation.
(23) The process according to any one of items 18 to 22, wherein said vector of step (ii) encodes RNA1 of a segmented Nepovirus, preferably TRSV.
(24) A kit comprising
   (a) the DNA molecule, DNA construct or vector according to item 15, and
   (b) a second DNA molecule or vector encoding proteins necessary for replicating and expressing the RNA molecule encoded by the DNA molecule, DNA construct or vector according to item 15.
(25) A process of producing a plus-sense single-stranded picornaviral RNA molecule in cells of a plant or plant cells, said RNA molecule comprising a segment encoding a movement protein, a segment encoding a coat protein, and a segment that comprises a guide RNA (gRNA), wherein said RNA molecule can be translated, in infected cells, to a polyprotein comprising the movement protein and the coat protein,

said process comprising providing a DNA molecule encoding the RNA molecule into said cells of said plant or said plant cells.
(26) The process according to item 25, further comprising providing a picornaviral RNA1 of said plus-sense single-stranded picornaviral RNA molecule, or a DNA vector encoding said RNA molecule, into said cells of a plant of plant cells.
(27) A process of infecting a plant with a genetically-modified picornavirus, comprising
   (A) providing, e.g. Agrobacterium-mediated, a first plant with a DNA molecule according to item 15 encoding said RNA molecule for expressing said RNA molecule in cells of said first plant, and
   (B) infecting said plant to be transfected, notably the plant to be infected, with said RNA molecule expressed in the first plant.
(28) The process according to item 27, further comprising collecting cell sap from said first plant, said sap containing said RNA molecule, and step (B) comprising rubbing or spraying said sap onto leaves using an abrasive.
(29) The process according to item 27 or 28, wherein two or more different plant species are infected in step (B).
(30) The plus-sense single-stranded RNA viral RNA molecule according to any one of items 1 to 14, wherein said gRNA is not operably linked to a subgenomic promoter, preferably said RNA molecule does not contain a subgenomic promoter.
(31) A plus-sense single-stranded RNA viral RNA molecule, comprising a segment encoding a movement protein, a segment encoding a coat protein, and a segment that comprises a guide RNA (gRNA),

wherein the RNA molecule is capable of directing sequence-specific binding of a CRISPR-endonuclease to a target DNA, and said RNA molecule can be translated, in infected cells, to a polyprotein comprising the movement protein and the coat protein, and

wherein said segment comprising the gRNA is preferably not operably linked to a subgenomic promoter.
(32) A plant, a plant tissue such as a callus or a shoot, a plant seed, or a plant cell containing the RNA molecule according to any one of claims 1 to 7 or the DNA molecule, DNA construct or vector according to claim 8.
(33) A plus-sense single-stranded RNA viral RNA molecule, wherein the RNA molecule is an RNA2 of a segmented nepoviral RNA comprising an inserted gRNA.
(34) A plus-sense single-stranded RNA viral RNA molecule, wherein the RNA molecule is an RNA2 of a tobacco ringspot virus (TRSV) comprising a gRNA sequence inserted by human deliberation.

The inventors have surprisingly found that gRNA can be successfully introduced into plants or plants cells using a picornaviral RNA molecule or vector, such as a tobacco ringspot viral RNA or vector, or a DNA copy thereof. This finding is surprising, since picornaviruses such as tobacco ringspot virus (TRSV) are bipartite viruses that express polyproteins in infected cells, and the gRNA is present as a fusion in the long RNA2 of the viral genome. The mechanisms as to how such gRNA delivered as part of a long viral genomic RNA can guide a CRISPR nuclease to a target nucleic acid is not yet understood. However, the delivery method for gRNA into plant cells is effective and allows gene editing such as cleavage of the target nucleic acid. The method of the invention is highly useful, as picornaviruses such as TRSV have a quite broad host plant range and therefore can be used for gene editing in a wide range of plants or plant cells.

### BRIEF DESCRIPTION OF THE DRAWINGS

FIG. 1 shows schematically T-DNA regions of TRSV viral vectors used in the Examples.

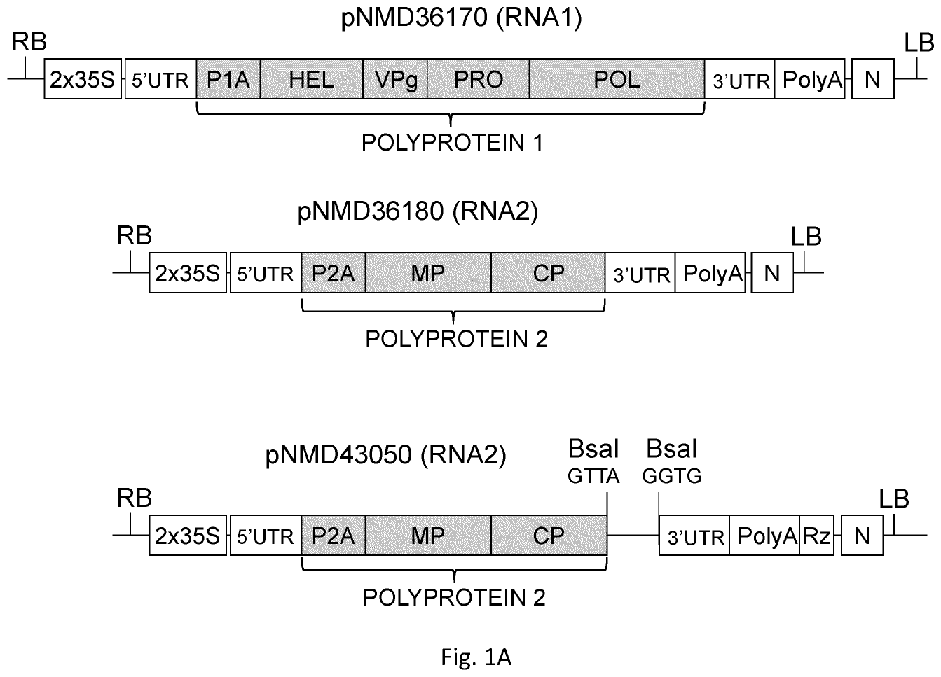

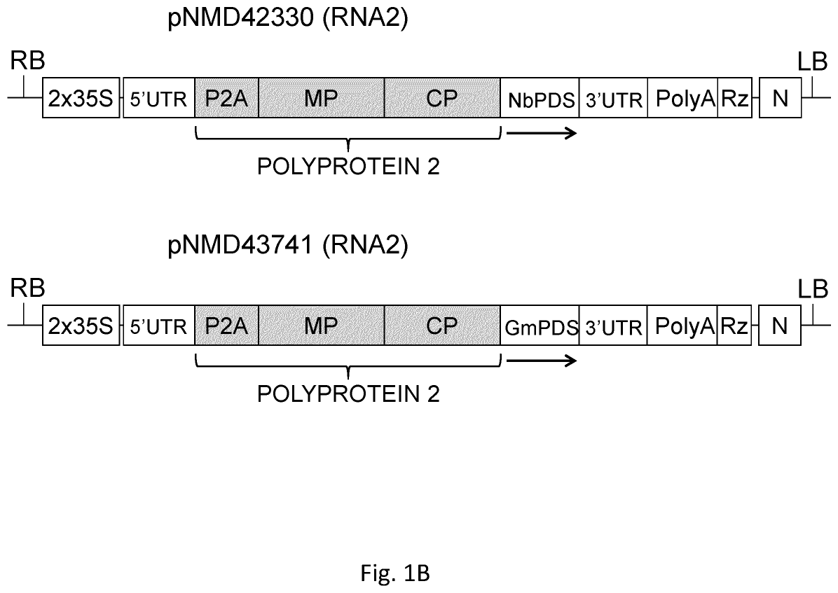

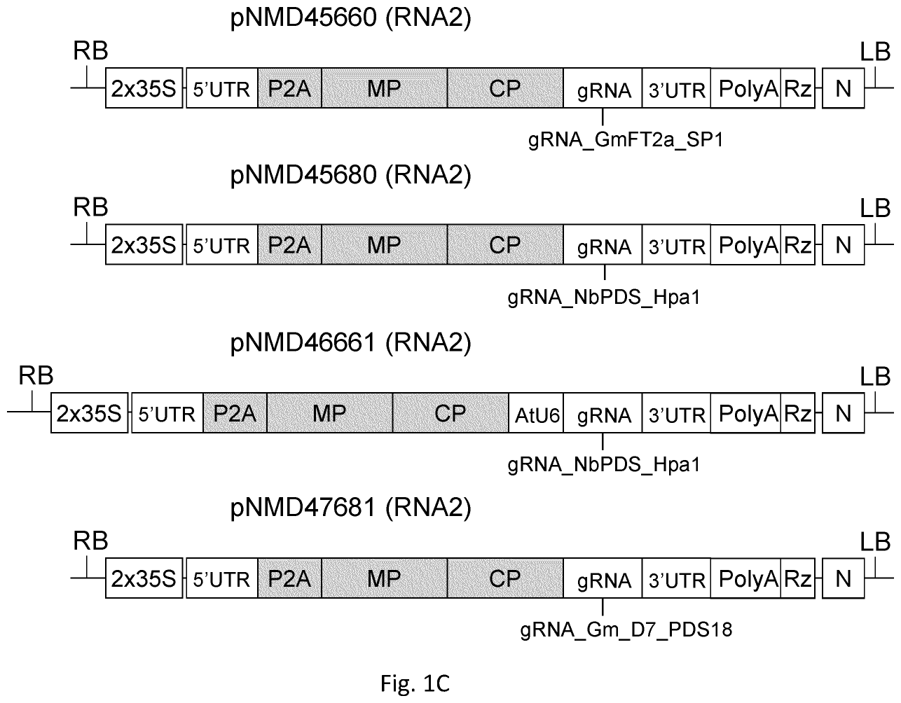

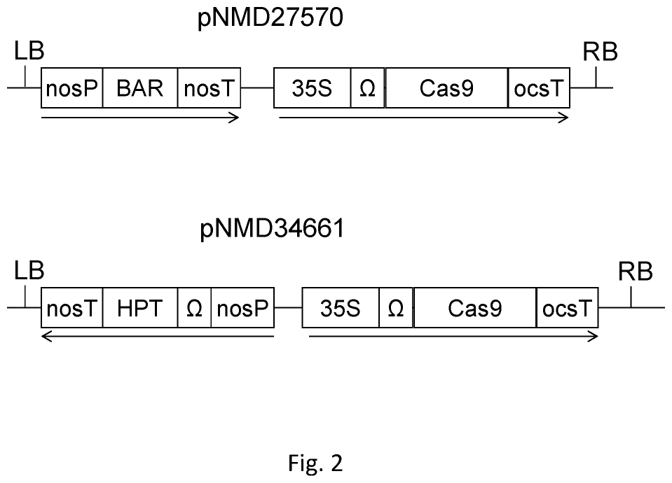

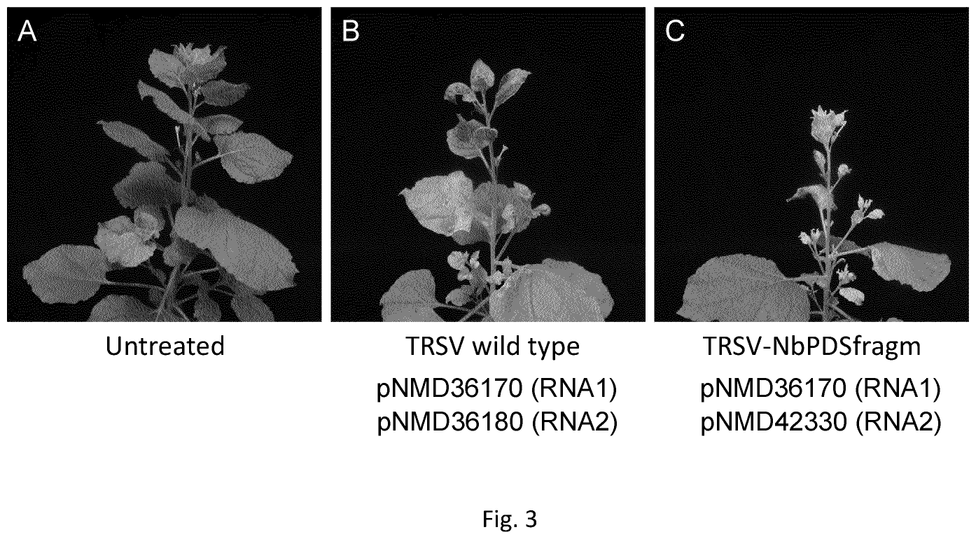

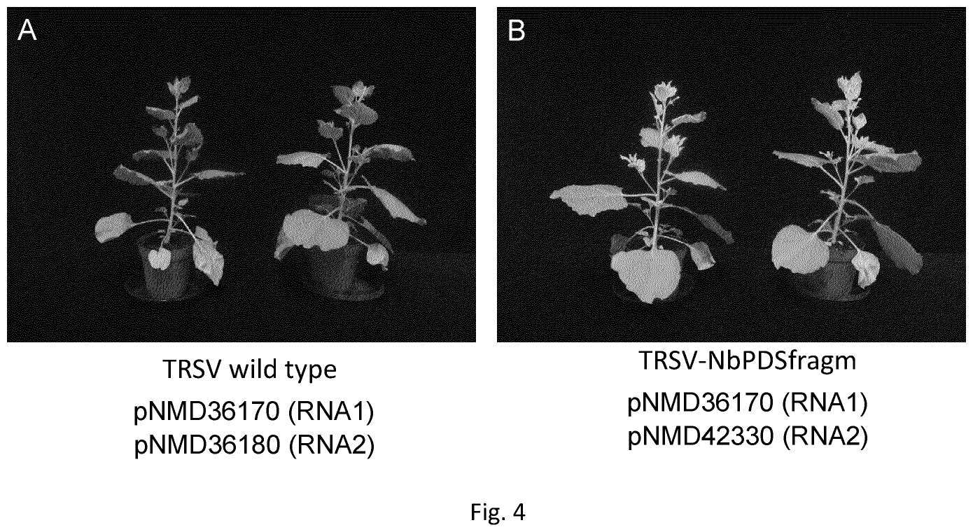

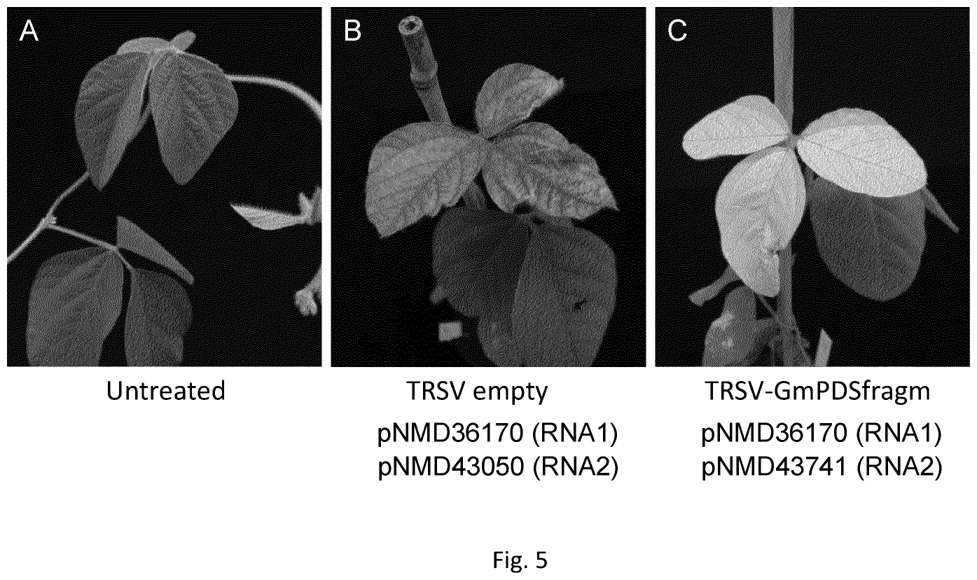

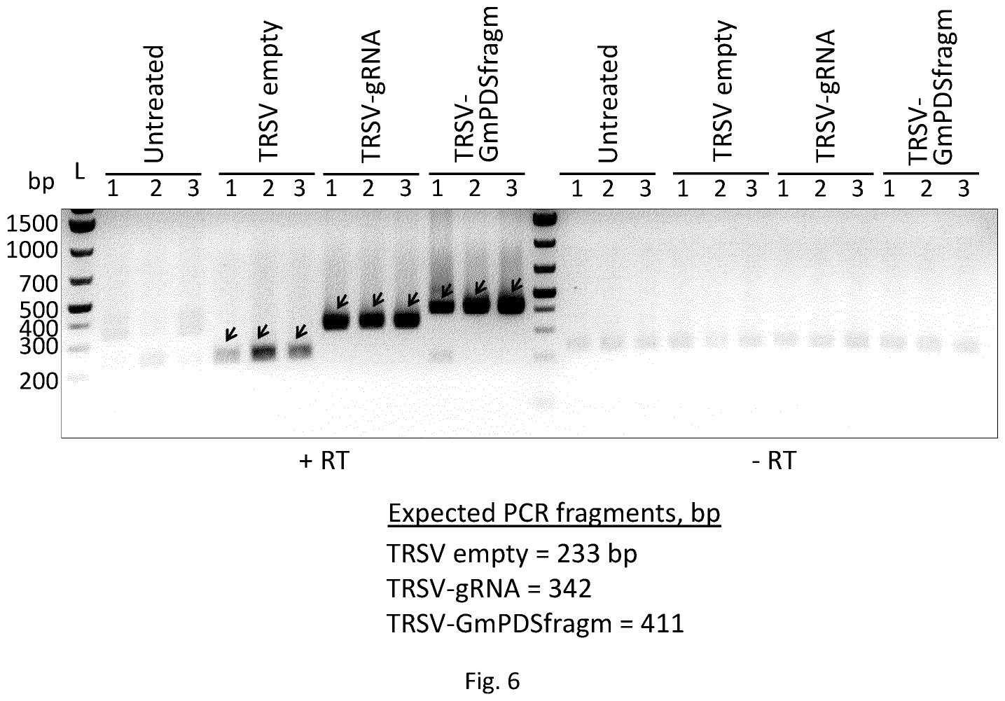

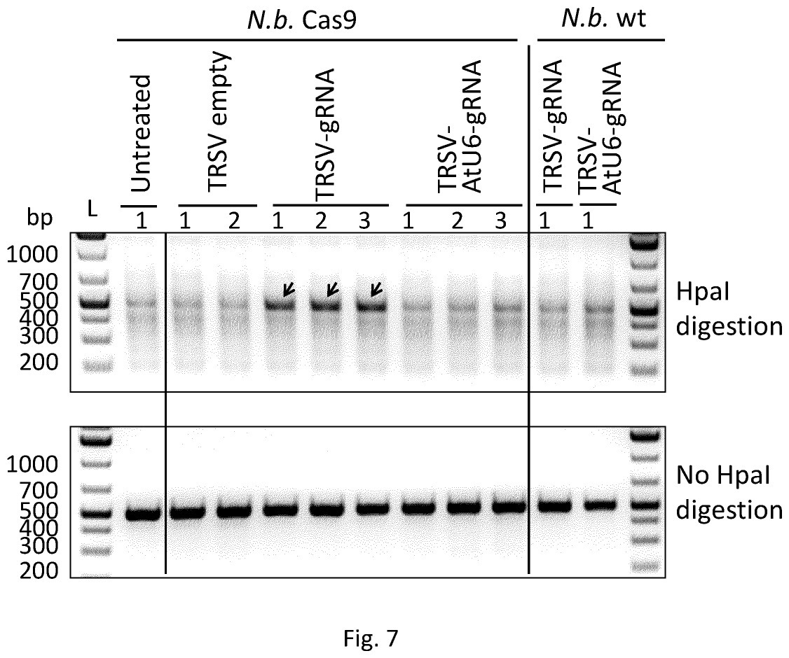

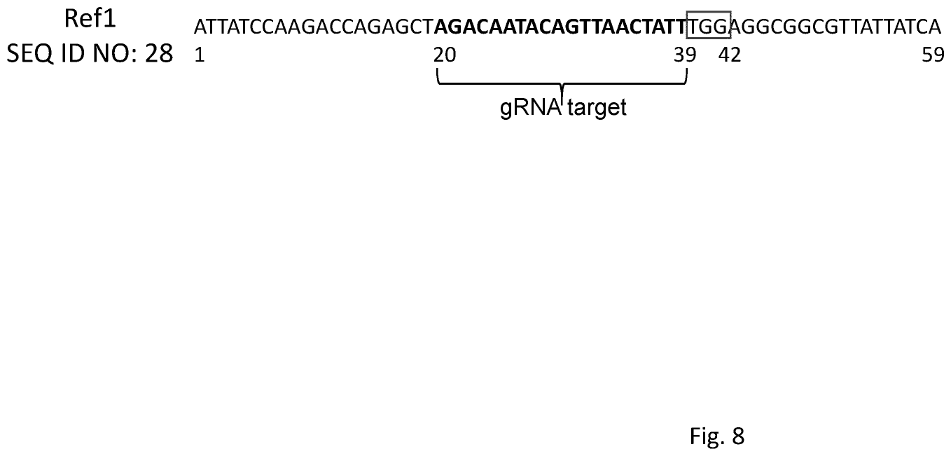

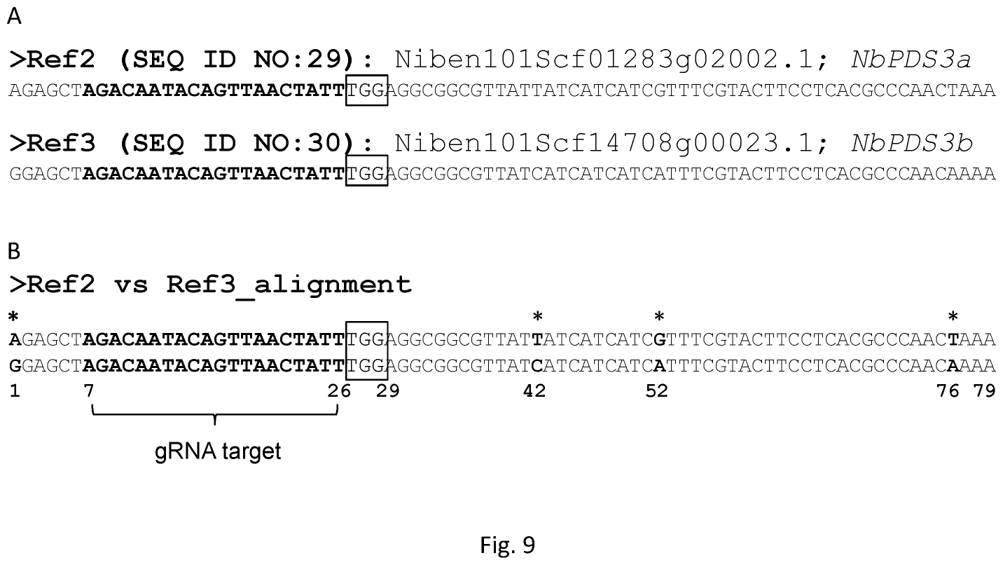

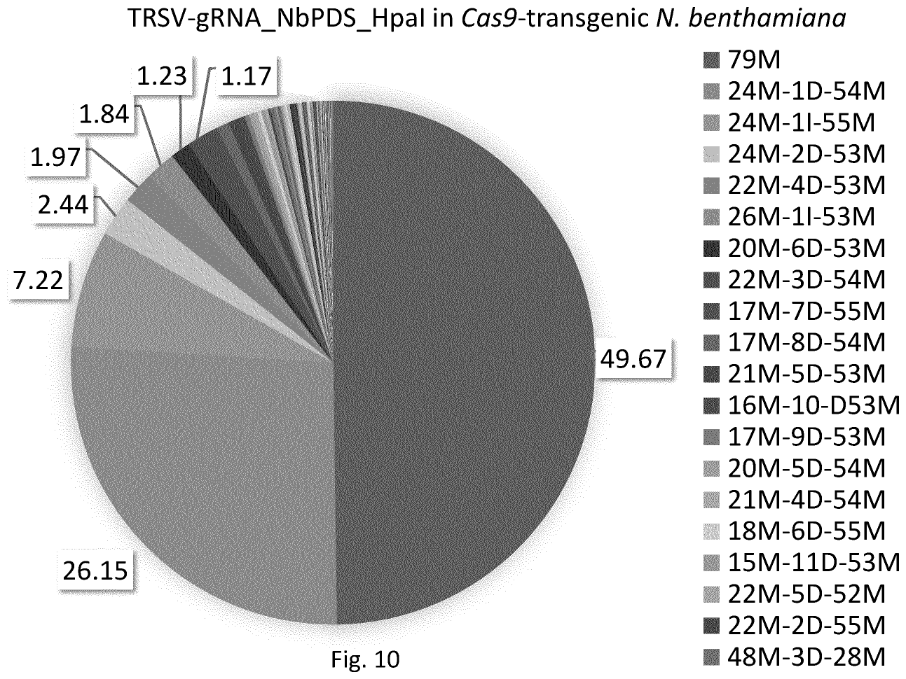

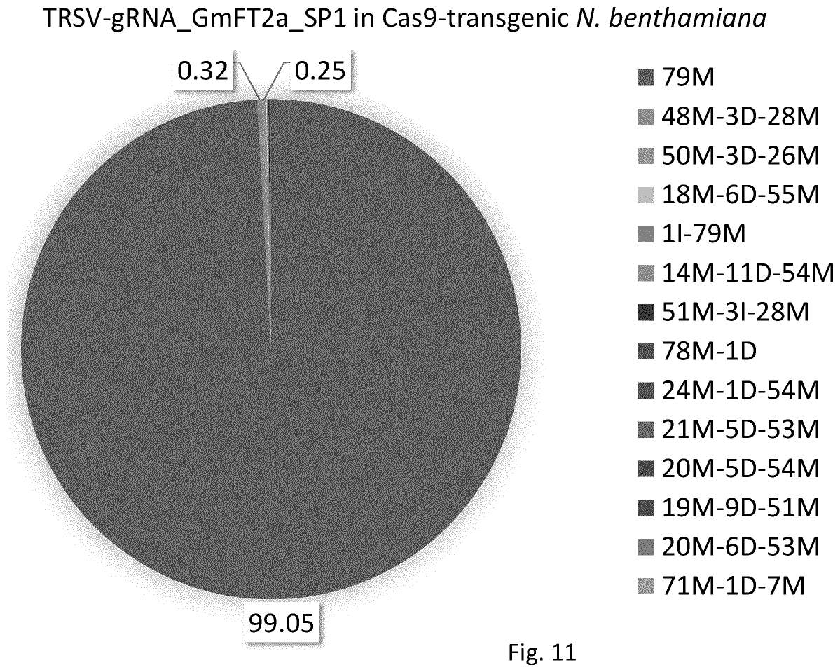

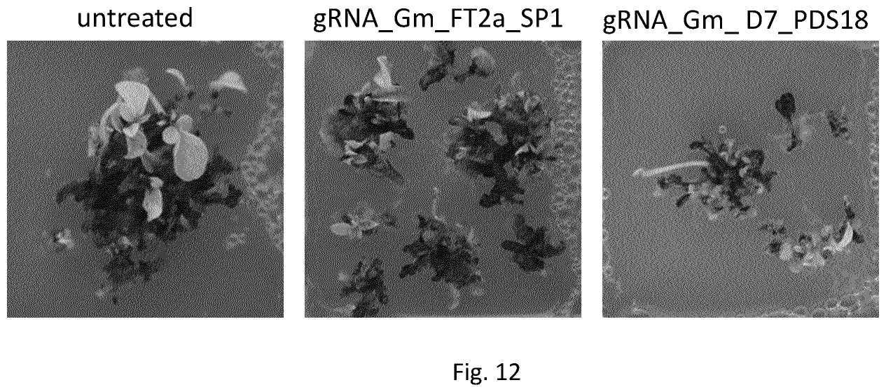

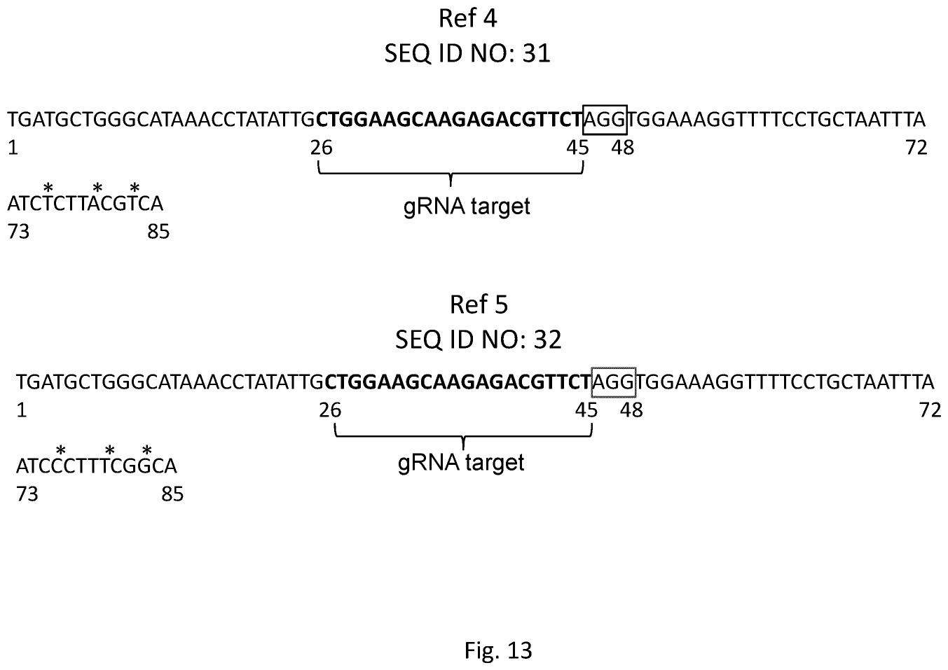

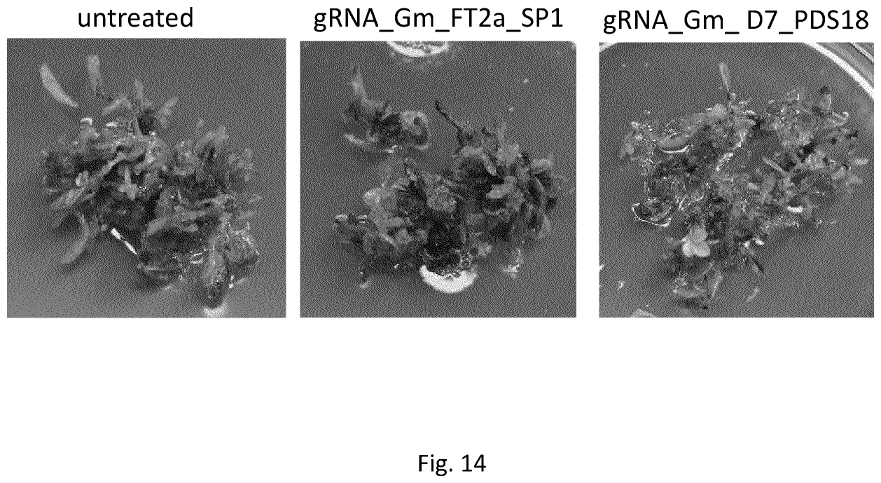

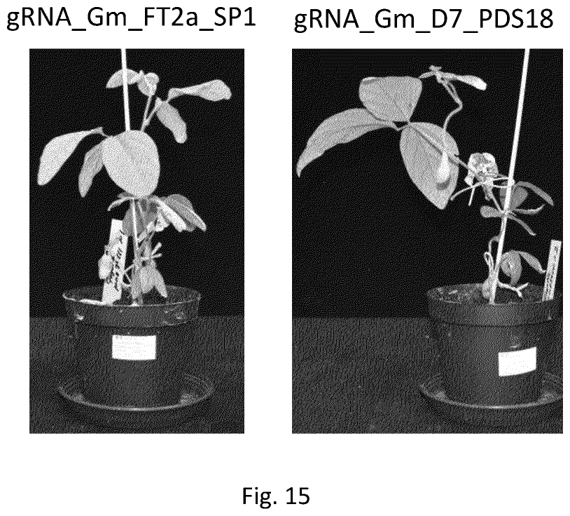

Constructs depicted in FIG. 1A carry next insertions: pNMD36170—intact RNA1 of TRSV, pNMD36180—intact RNA2 of TRSV, pNMD43050—RNA2 with BsaI(GTTA)-BsaI(GGTG) cloning site.

RB and LB stand for the right and left borders of T-DNA of binary vectors. 2x35S: double 35S promoter from cauliflower mosaic virus; 5′UTR: 5′-untranslated region of TRSV. POLYPROTEIN 1 or 2 stands for the TRSV polyprotein which is further cleaved into P1A: proteinase cofactor; HEL: helicase; VPg: genome-linked protein; PRO: protease; POL: RNA-dependent RNA polymerase (RNA1) or, respectively, P2A: protein involved in RNA-2 replication; MP: movement protein; CP: coat protein; 3′UTR: 3′-untranslated region of TRSV; PolyA: polyA region; Rz: ribozyme; N: nos terminator. POLYPROTEIN 1: polyprotein encoded by RNA1; POLYPROTEIN 2: polyprotein encoded by RNA2.

FIG. 1B shows RNA2 TRSV vectors with insertions of phytoene desaturase PDS gene fragments from Nicotiana benthamiana (pNMD42330) and soybean (pNMD43741).

FIG. 1C shows RNA2 TRSV vectors with insertions of gRNAs: GmFT2a_SP1 gRNA specific for Flowering Locus T 2a (FT2a) gene from soybean (pNMD45660); NbPDS_Hpa1 gRNA specific for phytoene desaturase (PDS) gene from Nicotiana benthamiana (pNMD45680 and pNMD46661); Gm_D7_PDS18 gRNA specific for phytoene desaturase (PDS11 and PDS18) genes from soybean (pNMD47681). gRNA stands for the single-guide RNA containing the sequence complementary to the target, and a tracrRNA; AtU6 stands for the U6 promoter from Arabidopsis thaliana.

FIG. 2 shows schematically T-DNA regions of transcriptional vectors used for the stable transformation of Nicotiana benthamiana (pNMD27570) and soybean (pNMD34661) plants.

RB and LB stand for the right and left borders of T-DNA of binary vectors. NosP: nos promoter, BAR: coding sequence of phosphinothricine N-acetyltransferase; 35S: 35S promoter; Ω: omega translational enhancer from Tobacco Mosaic Virus; Cas9: coding sequence of Cas9 endonuclease from Streptococcus pyogenes; ocsT: ocs terminator; HPT: coding sequence of hygromycin-phosphotransferase. Arrows show the direction of transcription.

FIG. 3 depicts Nicotiana benthamiana plants infected with TRSV using syringe infiltration with Agrobacteria carrying viral vectors. (A) Untreated control plant. (B) Plant infected with wild type TRSV (TRSV wild type, pNMD36170 and pNMD36180 constructs). (C) Plant infected with TRSV carrying the fragment of Nicotiana benthamiana PDS coding sequence in sense orientation (TRSV-NbPDSfragm, pNMD36170 and pNMD42330 constructs). Photos were taken 14 days post infiltration.

FIG. 4 depicts Nicotiana benthamiana plants infected with TRSV viral particles using leaf rubbing with a sap from infected plants of same species. (A) Plant infected with TRSV wild type virus (TRSV wild type, pNMD36170 and pNMD36180 constructs). (B) Plants infected with TRSV carrying the fragment of Nicotiana benthamiana PDS coding sequence in sense orientation (TRSV-NbPDSfragm, pNMD36170 and pNMD42330 constructs). Photos were taken 14 days post rubbing.

FIG. 5 shows soybean Glycine max ‘Bliskavitsa’ plants infected with TRSV viral particles using leaf rubbing with a sap from infected Nicotiana benthamiana plants. (A) Untreated control plant. (B) Plant infected with TRSV cloning vector without foreign insert (TRSV empty, pNMD36170 and pNMD43050 constructs). (C) Plant infected with TRSV carrying the fragment of soybean PDS coding sequence in sense orientation (TRSV-GmPDSfragm; pNMD36170 and pNMD43741 constructs). Pictures were taken 14 days post rubbing.

FIG. 6 shows RT-PCR analysis of TRSV vector stability in soybean Glycine max ‘Bliskavitsa’ plants mechanically inoculated with viral particles using leaf rubbing. Plant material was analyzed 21 days post rubbing. L: DNA ladder; Untreated: untreated plants (negative control); TRSV empty: plants infected with an empty TRSV vector for cloning (pNMD36170 and pNMD43050 constructs); TRSV-gRNA: plants infected with TRSV vector carrying the insertion of GmFT2a_SP1 gRNA (pNMD36170 and pNMD45660 constructs); TRSV-GmPDSfragm: plants infected with TRSV vector carrying the insertion of soybean PDS fragment in sense orientation (pNMD36170 and pNMD43741 constructs). PCR was performed using as a template either cDNA (+RT) or corresponding RNA (−RT). PCR fragments are pointed with arrows. 1, 2, 3: individual plants. Expected PCR fragment size is shown on the bottom; bp stands for base pairs.

FIG. 7 shows PCR-based analysis of Cas9-induced mutations in the PDS target site of Nicotiana benthamiana. Cas9-transgenic (N.b. Cas9) or wild type (N.b. wt) plants were used for agrobacterial infiltration with TRSV and TRSV viral vectors carrying NbPDS_Hpa1 gRNA. PCR was performed using either HpaI-digested genomic DNA (upper gel) or intact genomic DNA (lower gel) as a template.

L: DNA ladder; Untreated: untreated plant (negative control); TRSV empty: empty TRSV vector for cloning (pNMD36170 and pNMD43050 constructs); TRSV-gRNA: TRSV vector with NbPDS_Hpa1 gRNA insertion (pNMD36170 and pNMD45680); TRSV-AtU6-gRNA: TRSV vector with an insertion of NbPDS_Hpa1 gRNA with Arabidopsis U6 promoter (pNMD36170 and pNMD46661). 1, 2, 3: individual plants. Cleavage-resistant PCR fragments are shown with arrows. Expected size of uncleaved PCR fragment is 500 bp. Plant material was analyzed 16 days post infiltration.

FIG. 8 shows Reference Sequence 1 (Ref1, SEQ ID NO: 28), the fragment of Nicotiana benthamiana phytoene desaturase gene NbPDS3a (Niben101Scf01283g02002.1; SEQ ID NO: 28), nucleotide position 1201-1259). gRNA target is highlighted, the PAM sequence is shown in an open box.

FIG. 9 shows Reference Sequences of phytoene desaturase genes from Nicotiana benthamiana used for deep sequencing. (A) Individual sequence fragments of NbPDS3a (Niben101Scf01283g02002.1; Ref2; SEQ ID NO: 29) and NbPDS3b (Niben101Scf14708g00023.1; Ref3; SEQ ID NO: 30). (B) Alignment of Ref2 (top) and Ref3 (bottom) sequences. gRNA target (nucleotide position 7-26) is shown in highlighted form. PAM sequence (nucleotide position 27-29) is shown in an open box. Asterisks show variable nucleotides.

FIG. 10 shows frequency (% of total reads) of specific modifications in PDS target sequence and surrounding region of Cas9 transgenic Nicotiana benthamiana inoculated with TRSV vector carrying gRNA_NbPDS_HpaI (28 dpi). The legend on the right explains reference sequence patterns; M: number of matching nucleotides, D: number of deleted nucleotides; I: number of inserted nucleotides compared to reference sequence.

FIG. 11 shows frequency of specific modifications in PDS target sequence and surrounding region of Cas9 transgenic Nicotiana benthamiana inoculated with TRSV vector carrying gRNA_GmFT2a_SP1. For details, see the legend to FIG. 10.

FIG. 12 shows Cas9 transgenic shoots of soybean Glycine max ‘Fayette’ (pNMD34661, 35S-Cas9, hygR) used for spraying with TRSV viral particles in vitro (Experiment I). Photos were taken 9 weeks post spraying. Untreated: untreated shoots; gRNA_Gm_FT2a_SP1: shoots sprayed with TRSV viral particles carrying gRNA_Gm_FT2a_SP1 (pNMD36170 and pNMD45660 constructs); gRNA_Gm_D7_PDS18: shoots sprayed TRSV viral particles carrying gRNA_Gm_D7_PDS18 (pNMD36170 and pNMD47681 constructs).

FIG. 13 shows Reference Sequences of soybean GmPDS11 (Glyma. 11G253000; Ref 4, SEQ ID NO: 31) and GmPDS18 (Glyma. 18G003900; Ref 5, SEQ ID NO: 32) used for editing analysis. Target sequence for Gm_D7_PDS18 gRNA is highlighted; PAM sequence is shown in an open box; variable nucleotides are shown with asterisks.

FIG. 14 shows Cas9 transgenic shoots of soybean Glycine max ‘Fayette’ used for spraying with TRSV viral particles in vitro (Experiment II). Photos were taken 14 days post spraying. Untreated: untreated shoots; gRNA_Gm_FT2a_SP1: shoots sprayed with TRSV viral particles carrying gRNA_Gm_FT2a_SP1 (pNMD36170 and pNMD45660 constructs); gRNA_Gm_D7_PDS18: shoots sprayed TRSV viral particles carrying gRNA_Gm_D7_PDS18 (pNMD36170 and pNMD47681 constructs).

FIG. 15 shows Cas9 transgenic plants of soybean Glycine max ‘Fayette’ (pNMD34661, 35S-Cas9, hygR) infected with TRSV viral particles using rubbing of leaves. Photos were taken 16 days post rubbing. gRNA_Gm_FT2a_SP1: plant infected with TRSV viral particles carrying gRNA_Gm_FT2a_SP1 (pNMD36170 and pNMD45660 constructs); gRNA_Gm_D7_PDS18: plant infected with TRSV viral particles carrying gRNA_Gm_D7_PDS18 (pNMD36170 and pNMD47681 constructs).

### DETAILED DESCRIPTION OF THE INVENTION

Picornaviruses are plus-sense (+) single-stranded (ss) RNA viruses of the order Picornavirales. An example of picornaviruses for use in the invention is Tobacco ringspot virus (TRSV). The virus classification of TRSV is as follows:

Group: Group IV ((+)ssRNA)

Order: Picornavirales

Family: Secoviridae

Subfamily: Comovirinae

Genus: Nepovirus

Species: Tobacco ringspot virus

The RNA virus on which the RNA molecule of the invention is based has inter alia the following characteristics:
(+) ssRNA virus
bipartite genome (2 polyadenylated ss RNA (+): RNA1 and RNA2)
polyprotein synthesis
no subgenomic RNAs.

Preferably, the RNA virus on which the RNA molecule of the invention is based has a wide host range, infects soybean to produce bud blight, is transmittable by sap inoculation, is transmittable by pollen and by seed, has a high seed transmission rate in soybean (can reach 100%), and/or may be associated with embryonic tissue of seed. Preferably, the virus is from the order Picornavirales and infects soybean. More Preferably, the virus is Tobacco ringspot virus (TRSV).

The inventors of the present invention have found that the RNA2 of picornaviruses is suitable for delivering guide RNA (gRNA) into plant cells such that the gRNA can be used for guiding a CRISPR nuclease to a target nucleic acid, e.g. for introducing double strand breaks into the target RNA or target DNA or for other methods of gene editing.

For introducing gRNA into plant cells or cells of a plant, the invention provides a plus-sense single-stranded RNA viral RNA molecule. This RNA molecule comprises a segment encoding a movement protein (MP), a segment encoding a coat protein (CP), and a segment that comprises a guide RNA (gRNA), preferably a single-guide RNA (sgRNA). The RNA molecule can preferably be translated, in infected cells, more preferably in infected plant cells, to a polyprotein comprising the MP and the CP. The polyprotein may also comprise a segment that is a translation product of the gRNA. Optionally, the RNA molecule also contains, generally upstream of the MP encoding segment, a segment encoding a protease (such as P2A) that can proteolytically cleave the polyprotein expressed from the RNA molecule in infected cells. Generally, the polyprotein is expressed from a single open reading frame from the RNA molecule of the invention. Generally, the RNA molecule does not contain a subgenomic promoter that would, in infected cells, allow synthesis of RNA comprising the gRNA segment by an RNA-dependent RNA polymerase (RdRP). Preferably, the RNA molecule does not contain a subgenomic promoter that would allow production, in infected cells, of RNA comprising the MP segment, the CP segment and the gRNA segment.

The RNA molecule of the invention may be based on RNA2 of a bipartite picornavirus, i.e. member of virus order Picornavirales. Here, “be based on” means that it contains genetic elements of the RNA2 of the picornavirus. Accordingly, the RNA molecule contains, in 5′- to 3′-direction, the movement protein (MP) encoding sequence and a coat protein (CP) encoding sequence. The RNA molecule generally further contains, preferably on the 5′-side of the MP encoding sequence, a P2A encoding sequence that encodes a protease capable of cleaving the polyprotein translated from the RNA molecule. The RNA molecule may further comprise, optionally after replication in plant cells, a VPg (viral protein genome-linked) peptide at the 5′-terminus and/or a polyA tail at the 3′-end.

The length of the RNA molecule of the invention is generally at least 3500 nucleotides, preferably at least 4000 nucleotides. The length of the RNA molecule of the invention may range from 5000 to 6000 nucleotides.

The RNA molecule of the invention does generally not comprise a segment encoding an RNA-dependent RNA polymerase (RdRP) or replicase. The terms “RdRP” and “replicase” are used synonymously herein. The RdRP for replicating the RNA molecule of the invention inside a cell may be expressed from a different DNA molecule or RNA molecule. For example, the RNA1 molecule of a bipartite virus may encode an RdRP for replicating the RNA molecule of the invention in cells. Preferably, the RdRP is expressed from the RNA1 of a bipartite picornavirus. In one embodiment, the RdRP is expressed from the RNA1 of the tobacco ringspot virus (TRSV). In another embodiment, the RdRP is expressed from a T-DNA inserted into the genome of a host cell through transformation with Agrobacterium.

In a preferred embodiment, the RNA molecule of the invention does not encode an RdRP as described above and does not contain a subgenomic promoter as described above. In another preferred embodiment, the RNA molecule of the invention does not encode an RdRP and does not contain a subgenomic promoter that would allow production, by the RdRP, of RNA of a length more than 100 nucleotides shorter than that of the RNA molecule.

As mentioned above, the RNA molecule comprises a gRNA as a component of a CRISPR system. As is known to the skilled person in the field of CRISPR-Cas-based gene editing, the gRNA has complementarity to a target nucleic acid (generally target DNA) and has the ability to bind to the effector protein such as a nuclease that may be used for cleaving the target nucleic acid. The nuclease may be Cas9 or Cpf1. In some embodiments, the gRNA comprises a guide sequence linked to a direct repeat sequence. The guide sequence provides the complementarity to a target nucleic acid for guiding the effector protein, such as the nuclease, to the target sequence. The direct repeat sequence generally provides portions that allow binding of the gRNA to a CRISPR effector protein (e.g. nuclease) as, for example, in a tracrRNA. Otherwise, the gRNA is not particularly limited. The gRNA may be a single guide RNA (sgRNA), i.e. it may comprise a transactivating RNA (tracrRNA) required for certain CRISPR-Cas systems, such as the Type II CRISPR-Cas9 system.

A gRNA comprises a sequence stretch complementary to the target DNA and, if required, a trans-activating CRISPR RNA (tracrRNA). The sequence stretch complementary to the target DNA may have a length of from 19 to 22 contiguous nucleotides, preferably from 20 to 21 nucleotides. The succession of these elements depends on the type of CRISPR-Cas-system used.

For use of Cas9 or a Cas effector protein of a class 2, Type II CRISPR-Cas-system, the gRNA is generally a sgRNA that comprises in 5′ to 3′-direction a sequence stretch complementary to the target DNA and a trans-activating CRISPR RNA (tracrRNA). For example, in FIG. 8, the DNA sequence targeted by the gRNA is indicated as “gRNA target”. In the Examples, the tracrRNA is provided by the gRNA scaffold sequence (Mali et al., 2013). The use of CRISPR-Cas systems is generally known to the skilled person. A Cas effector protein used in the invention does not need to have the nuclease activity of a natural Cas protein such as Cas9, but may be a variant having the nuclease activity fully or partially removed by genetic engineering; alternatively or additionally, other activities may be added to a Cas effector protein e.g. by making a fusion protein of the Cas effector protein with another protein having a desired function when targeted to the target sequence.

The RNA molecule may comprise:
(o) optionally a picornaviral 5′-UTR (untranslated region),
(i) a segment encoding a picornaviral P2A protein (“P2A segment”),
(ii) a segment encoding a picornaviral movement protein (“MP segment”),
(iii) a segment encoding a picornaviral coat protein (“CP segment”),
(iv) a segment comprising a guide RNA (gRNA), such as a single guide RNA (sgRNA), and
(v) optionally a picornaviral 3′-UTR and optionally a polyA tail.

The segment comprising the gRNA is also referred to herein as “gRNA segment”. The 5′-UTR may be present at the 5′-end of said RNA molecule. The 3′-UTR may be present downstream (3′) of segments (o) and (i) to (iv). Generally, the RNA molecule contains a polyA tail downstream of the 3′-UTR. The polyA tail may comprise from 10 to 50, preferably from 20 to 40 contiguous adenine (A) bases. The 5′-UTR and the 3′-UTR are generally taken from the same picornavirus from which items (i) to (iii) are taken. The order of items (i)-(iii) is not particularly limited. However, items (ii)-(iii) are generally in this order in 5′ to 3′-direction given (i.e. item (iii) being on the 3′-side of item (ii)). In this embodiment, items (i)-(iii) are present in this order. The position of the segment (iv) comprising the gRNA in the RNA molecule is not particularly limited. The gRNA segment may, for example, be located between the MP segment and the CP segment or between the CP segment and the 3′-UTR. It may also be part of the 3′-UTR. Further, the gRNA segment may be within the MP or CP segment, respectively. A ribozyme (referred to as “Rz” in the figures) may be added at the 3′-end of the RNA molecule for achieving a well-defined 3′-end of the RNA molecule upon replication in infected cells. The UTRs may contain promoters for replicating the RNA molecule by an RdRP, as is generally known to a person skilled in the art of plant viral expression systems.

The RNA molecule is preferably based on the RNA2 of a virus of Family Secoviridae. The RNA molecule may thus comprise
(o) optionally a secoviral 5′-UTR,
(i) optionally a segment encoding a secoviral P2A protein (“P2A segment”),
(ii) a segment encoding a secoviral movement protein (“MP segment”),
(iii) a segment encoding a secoviral coat protein (“CP segment”),
(iv) a segment comprising a guide RNA (gRNA), such as a single guide RNA (sgRNA), and
(v) optionally a secoviral 3′-UTR and optionally a polyA tail.
   Regarding the order in 5′ to 3′-direction of items (o)-(iii) and (v) and the position of the segment comprising the gRNA, what has been said above applies analogously.

More preferably, the RNA molecule is preferably based on the RNA2 of a virus of subfamily Comovirinae. The RNA molecule may thus comprise
(o) optionally a 5′-UTR a virus of subfamily Comovirinae,
(i) optionally a segment encoding a P2A protein (“P2A segment”) of a virus of subfamily Comovirinae,
(ii) a segment encoding a movement protein (“MP segment”) of a virus of subfamily Comovirinae,
(iii) a segment encoding a coat protein (“CP segment”) of a virus of subfamily Comovirinae, and
(iv) a comprising a guide RNA (gRNA), such as a single guide RNA (sgRNA), and
(v) optionally a 3′-UTR of a virus of subfamily Comovirinae and optionally a polyA tail.
   Regarding the order in 5′ to 3′-direction of items (o)-(iii) and (v) and the position of the segment comprising the gRNA, what has been said above applies analogously.

Even more preferably, the RNA molecule is preferably based on the RNA2 of a virus of genus Nepovirus. The RNA molecule may thus comprise
(o) optionally a secoviral 5′-UTR,
(i) optionally a segment encoding a nepoviral P2A protein (“P2A segment”),
(ii) a segment encoding a nepoviral movement protein (“MP segment”),
(iii) a segment encoding a nepoviral coat protein (“CP segment”), and
(iv) a segment comprising a guide RNA (gRNA), such as a single guide RNA (sgRNA), and
(v) optionally a nepoviral 3′-UTR and optionally a polyA tail.
   Regarding the order in 5′ to 3′-direction of items (i)-(iii) and (v) and the position of the segment comprising the gRNA, what has been said above applies analogously.

Even more preferably, the RNA molecule is preferably based on the RNA2 of tobacco ringspot virus. The RNA molecule may thus comprise
(o) optionally a TRSV 5′-UTR,
(i) optionally a segment encoding a TRSV P2A protein (“P2A segment”),
(ii) a segment encoding a TRSV movement protein (“MP segment”),
(iii) a segment encoding a TRSV coat protein (“CP segment”), and
(iv) a segment comprising a guide RNA (gRNA), such as a single guide RNA (sgRNA), and
(v) optionally a TRSV 3′-UTR and optionally a polyA tail.
   Regarding the order in 5′ to 3′-direction of items (i)-(iii) and (v) and the position of the segment comprising the gRNA, what has been said above applies analogously.

The RNA molecule can be translated, in infected cells, to a polyprotein comprising the proteins of items (i) to (iii). The polyprotein may thus be expressed from a single open reading frame of the RNA molecule. The polyprotein may also comprise a segment encoded by the gRNA. The RNA molecule is preferably capable of replicating in a prokaryotic or eukaryotic cell, e.g. in the presence of a picornaviral RNA polymerase (or RdRP), to form replicated RNA comprising a VPg group at its 5′-end and, generally, a poly-A tail at its 3′-end. Replication in cells generally requires the presence of a picornaviral RNA polymerase to form replication product of the RNA molecule. The replication product generally comprises segments (i)-(iv) above, the 5′-UTR, the 3′-UTR, and a VPg group at its 5′-end and a poly-A tail at its 3′-end.

For achieving replication of the RNA molecule in plant cells or cells of a plant, the cells are preferable provided with the RNA1 of the bipartite picornavirus. The RNA1 encodes the structural proteins of the picornavirus, such as a helicase, the VPg, and a polymerase, preferably the RNA1 encodes the P1A protein, the helicase, VPg, Pro, and the polymerase. The structural proteins of the RNA1, notably the polymerase, can then replicate the RNA molecule. Preferably, the structural proteins of the RNA1 can also replicate the RNA1. One or more proteins of RNA1 can, alternatively, be provided to plant cells or cells of a plant by making transgenic plants encoding such proteins such that they can be expressed.

The RNA molecule of the invention can be introduced into plant cells or cells of a plant by a variety of methods that are commonly known in the art. Examples are electroporation, microinjection, and particle bombardment. Whichever method is used, it is generally preferred that the RNA1 that provides the functions and proteins for replication of the RNA molecule is introduced into the plant cells or cells of a plant by the same method, preferably in parallel or simultaneously, i.e. by co-transfection. In co-transfection, a mixture of RNA1 and the RNA molecule of the invention, or a mixture of DNA molecules encoding RNA1 and the RNA molecule of the invention, is provided to plant cells or cells of a plant. Where DNA molecules are provided to plant cells or cells of a plant by Agrobacterium-mediated transfection (see further below), the plant cells or cells of a plant may be treated with mixture of Agrobacterium strains, one strain containing a DNA encoding RNA1 and one strain encoding the RNA molecule of the invention.

Since RNA is generally more difficult to handle than DNA, the RNA molecule of the invention is preferably introduced into plant cells or cells of a plant by introducing into plant cells or cells of a plant a DNA molecule that can be transcribed in said cells to said RNA molecule. RNA1 may be introduced into the plant cells or cells of a plant analogously and preferably in parallel or simultaneously, i.e. by co-transfection.

Therefore, the invention also provides a DNA molecule encoding the RNA molecule of the invention. For enabling transcription of the DNA molecule to the RNA molecule in cells, the DNA molecule preferably comprises regulatory elements for transcription. In a preferred embodiment, the DNA molecule comprises, in 5′ to 3′ direction, a transcription promoter active in plant cells, a sequence encoding the RNA molecule, and optionally a terminator sequence. The invention also provides a second DNA molecule that encodes RNA1. For enabling transcription of the second DNA molecule in cells, the second DNA molecule comprises regulatory elements therefor. Preferably, the second DNA molecule comprises, in 5′ to 3′ direction, a transcription promoter active in plant cells, a sequence encoding the RNA1, and optionally a terminator sequence. Suitable promoters are described below.

Various methods for introducing the DNA molecule and optionally also the second DNA molecule into plant cells or cells of a plant are known, and examples are electroporation, microinjection, and particle bombardment. However, the preferred method of introducing the DNA molecule of the invention, and optionally the second DNA molecule, into plant cells or cells of a plant is Agrobacterium-mediated transfection. Agrobacterium-mediated transfection is well-established in the field of plant biotechnology.

For Agrobacterium-mediated transfection, the DNA molecule of the invention may be a plasmid containing in T-DNA a DNA construct encoding the RNA molecule of the invention. The Agrobacterium strain may belong to the species Agrobacterium tumefaciens or Agrobacterium rhizogenes that are commonly used for plant transformation and transfection and which are known to the skilled person from general knowledge. The Agrobacterium strain to be used in the processes of the invention may comprise a DNA molecule (Ti-plasmid or binary vector) as said DNA molecule. Said DNA molecule comprises a DNA construct encoding the RNA molecule of the invention. Said DNA construct also generally comprises (as described above for the DNA molecule) a transcription promoter active in plant cells for transcription of the sequence encoding the RNA molecule, a sequence encoding the RNA molecule, and optionally a terminator sequence. The DNA construct is typically present in T-DNA of the plasmid for introduction of the nucleic construct into plant cells by the secretory system of the Agrobacterium strain. On at least one side or on both sides, the nucleic acid construct is flanked by a T-DNA border sequence for allowing transfection of said plant(s) and introduction into plant cells or cells of a plant of said DNA construct. Preferably, said DNA construct is present in T-DNA and flanked on both sides by T-DNA border sequences. Herein, the term “DNA construct” means a recombinant construct comprising a DNA sequence encoding the RNA molecule of the invention.

The DNA construct may be present in T-DNA of a Ti-plasmid or binary vector of the Agrobacterium strain. Ti-plasmids or binary vectors may contain a selectable marker outside of said T-DNA for allowing cloning and genetic engineering in bacteria. However, the T-DNA that is transferred into cells of said plant does preferably not contain a selectable marker that would, if present, allow selection of plant or plant cells containing said T-DNA. Examples of selectable marker genes that should, in this embodiment, not be present in T-DNA of the Ti-plasmid or binary vectors are an antibiotic resistance gene or a herbicide resistance gene. The process of the invention preferably makes use of transient transfection, In this embodiment, the process of the invention does not comprise a step of selecting for plant cells or plants having incorporated the nucleic acid molecule of the invention by using such antibiotic resistance gene or a herbicide resistance gene. Accordingly, no antibiotic resistance gene or a herbicide resistance gene needs to be incorporated into said plants.

The DNA construct comprises a DNA sequence encoding the RNA molecule of the invention such that the latter is expressible in plant cells. For this purpose, the DNA sequence of interest may be, in said DNA construct, under the control of a promoter active in plant cells. Agrobacterium-mediated gene transfer and vectors therefor are known to the skilled person, e.g. from the references cited herein or from text books on plant biotechnology such as Slater, Scott and Fowler, Plant Biotechnology, second edition, Oxford University Press, 2008. Agrobacterium strains usable in the invention are those that are generally used in the art for transfecting or transforming plants. Generally, binary vector systems and binary strains are used, i.e. the vir genes required for transfer of T-DNA into plant cells on the one hand and the T-DNA on the other hand are on separate plasmids. Examples of usable Agrobacterium strains are given in the article of Hellens et al., Trends in Plant Science 5 (2000) 446-451 on binary Agrobacterium strains and vector systems. In the context of a binary Agrobacterium strain, the plasmid containing the vir genes is referred to as “vir plasmid” or “vir helper plasmid”. The plasmid containing the T-DNA to be transfected is the so-called binary vector that may be a “DNA molecule” or “vector” of the invention. The term “strain” or “Agrobacterium strain” relates to components of the Agrobacterium other than the binary vector. Thus, a binary Agrobacterium strain not containing a binary vector and a strain obtained after introduction of a binary vector are referred to by the same strain name.

Accordingly, the invention also provides an Agrobacterium cell containing the DNA molecule of the invention. Notably, the invention provides Agrobacterium cell comprising a plasmid comprising in T-DNA the construct encoding the RNA molecule of the invention.

Agrobacterium-mediated transection of plant cells or cells of a plant allows co-transfection of plant cells or cells of a plant. The plant cells or cells of a plant are at least transfected with said DNA molecule. Preferably, the plant cells or cells of a plant are also transfected with said second DNA molecule that encodes RNA1 of the picornavirus. Co-transfection by Agrobacterium can be achieved by preparing two different Agrobacterium strains, a first one that contains a plasmid (Ti plasmid or binary vector), construct or vector encoding the RNA molecule and a second Agrobacterium strain containing the second plasmid encoding RNA1. Suspensions of these Agrobacterium strains may be separately grown and mixed prior to transfection. The suspension of agrobacteria may be produced as follows. The DNA molecule or vector containing the DNA construct may be transformed into the Agrobacterium strain and transformed Agrobacterium cultures may be grown optionally under application of selective pressure for maintenance of said DNA molecule. In one method, the Agrobacterium strain to be used in the processes of the invention is then inoculated into a culture medium and grown to a high cell concentration. Larger cultures may be inoculated with small volumes of a highly concentrated culture medium for obtaining large amounts of the culture medium. Agrobacteria are generally grown up to a cell concentration corresponding to an OD at 600 nm of at least 1, typically of about 1.5. Such highly concentrated agrobacterial suspensions are then diluted to achieve the desired cell concentration. For diluting the highly concentrated agrobacterial suspensions, water is used. The water may contain a buffer or salts. The water may further contain the surfactant or wetting agent. Alternatively, the concentrated agrobacterial suspensions may be diluted with water, and any additives such as the surfactant and the optional buffer substances are added after or during the dilution process. Separately produced suspensions for co-transfection may then be mixed and the mixed suspension be used for transfecting plant cells or cells of a plant.

If plant cells in cell culture are to be transfected, an Agrobacterium suspension may be added to the plant cell culture. If selected parts of a plant such as plant leaves are to be transfected, the generally known agroinfiltration may be used, whereby a pressure difference is used to insert the Agrobacterium suspension into plant tissue. For example, a needle-less syringe containing the Agrobacterium suspension may be used to press an Agrobacterium suspension into plant tissue. In another agroinfiltration method, an entire plant or major parts of a plant is dipped upside down into an Agrobacterium suspension, a vacuum is applied and then quickly released, whereby an Agrobacterium suspension is inserted into plant tissue. In another embodiment, plants or plant parts are sprayed with a suspension containing cells of an Agrobacterium strain, which is well suitable for large scale applications to many plants such as to plants on a farm field. Such spray transfection processes are described in detail in WO2012/019660.

The invention also provides a kit comprising
(a) the DNA molecule, DNA construct or vector according to the invention, and
(b) a second DNA molecule or vector encoding proteins necessary for replicating and expressing the RNA molecule encoded by the DNA molecule, DNA construct or vector according to (a). The kit may be a kit of Agrobacterium strains, a first strain containing the DNA molecule, DNA construct or vector according to (a) and a second Agrobacterium strain containing an item according to (b). The kit may also be a kit of Agrobacterium cultures or batches, a first Agrobacterium culture or batch containing the DNA molecule, DNA construct or vector according to (a) and a second Agrobacterium culture or batch containing the second DNA molecule or vector according to (b).

The invention further provides a process of sequence-specifically affecting a target DNA or of conducting gene editing in a plant or a plant cell, comprising the following steps:
(i) transfecting said plant with the RNA molecule of the invention or with the DNA molecule of the invention and
(ii) transfecting said plant with a vector encoding proteins necessary for replicating and expressing said RNA,

wherein said gRNA is capable of hybridizing with said target DNA and wherein steps (i) to (ii) can be performed in any order or simultaneously.

The invention also provides a plant, a plant tissue such as a callus or a shoot, a plant seed, or a plant cell containing the RNA molecule of the invention. The plant tissue such as a callus or a shoot may be regenerated from a plant comprising the RNA molecule of the invention. The plant, plant tissue, plant seed, or plant cell may further comprise one or more proteins necessary for replicating and expressing said RNA molecule. The invention further provides a plant, a plant tissue such as callus or a shoot, a plant seed, or a plant cell containing a (first) DNA molecule encoding the RNA molecule of the invention. The plant, plant tissue, plant seed, or plant cell may further comprise a second DNA molecule encoding one or more proteins necessary for replicating and expressing the RNA molecule encoded by the first DNA molecule.

The plants of the invention may by selected from the families, genera or species listed below.

For sequence-specifically affecting a target DNA, such as gene editing, a protein capable of binding the gRNA and target DNA is required in the plant or in the plant cells wherein the affecting of the target DNA is to be carried out. Such protein is referred to herein as effector protein or CRISPR effector protein. The effector protein may be a CRISPR nuclease such as Cas9 or Cpf1 or a modified version thereof that can exert the desired activity on the target DNA (see further below). Therefore, a plant or plant cell is provided with the effector protein. There are various possibilities to provide a plant or plant cell with the effector protein. For example, the plant or plant cell may be transgenic for a gene encoding the effector protein, such that the effector protein is expressed in the plant or plant cell. Methods of generating transgenic plants expressing a protein of interest such as the effector protein of the invention are known in the art (see e.g. Slater, Scott and Fowler, Plant Biotechnology, second edition, Oxford University Press, 2008). Such methods may involve transforming tissue of a plant with heterologous DNA encoding the effector protein, selecting cells or tissue having incorporated the heterologous DNA, and regenerating a plant from the transgenic tissue. Another method of providing a plant or plant cell with the effector protein is co-transfecting the plant or plant cell with the DNA molecule or vector encoding the RNA molecule of the invention and a vector comprising a gene encoding the effector protein. This vector may be a third DNA molecule of the invention. Co-transfection may further involve transfection with said second DNA molecule. Co-transfection may be made by Agrobacterium-mediated transfection as described above. In a preferred embodiment, the RNA molecule of the invention also encodes the effector protein. Alternatively, the vector of step (ii) that encodes the proteins necessary for replicating the RNA molecule also encodes the effector protein. These embodiments have the advantage that the effector protein and no additional transfection or transformation step is required for providing the plant or plant cell with the effector protein.

Other methods of providing a plant or plant cell with the effector protein are microinjection of the effector protein into cells or particle bombardment.

The process of sequence specifically affecting a target DNA may affect the target DNA in many different ways. In this regard, the invention is not limited and any of the applications of CRISPR-Cas systems known may be used in the invention.

To sequence-specifically modify a target DNA, the effector protein may be a CRISPR nuclease, for example Cas9. The effector protein having bound gRNA (such as a sgRNA) can scan in the plant cell the target DNA to recognize a target sequence adjacent to a Proto-spacer Adjacent Motif (so-called PAM-sequence). When the PAM-sequence is detected on the target DNA, the effector protein binds to it and may unwind the DNA. Subsequently, the distal part of the gRNA, which is complexed with the effector protein, can hybridize with the unwound target DNA to identify the target site as determined by the gRNA. When about 20 contiguous nucleotides of the distal end of the gRNA have successfully hybridized with the separated DNA strand, the effector protein may exert its function. If the effector protein is a nuclease, the nuclease function may be activated. The nuclease may then cleave the target DNA near the PAM sequence. The pattern of the DNA cleavage depends on the properties of the nuclease. A CRISPR nuclease usually introduces double strand breaks (DSBs). The DSBs may have blunt ends (e.g. in the case of Cas9). If DSBs with sticky ends are desired, Cpfl may be used as the CRISPR nuclease or effector protein. In a further alternative, the target DNA may be nicked, i.e. only one of the strands of the target DNA is cleaved. Nicking may be achieved by using a CRISPR nuclease as effector protein having one of the two nuclease domains of a natural CRISPR nuclease inactivated by mutation.

As strand breaks in the target DNA are potentially dangerous for the host cell, it generally attempts to repair the nicked or cleaved DNA. As is generally known (see review articles cited in the background section), the cell can may employ one of two different repair mechanisms: non-homologous end joining (NHEJ) or homology-directed repair (HDR). DNA repair through NHEJ glues the broken ends of a double strand break (DSB) back together. As this process is error-prone, short insertions and/or deletions (indels) can be introduced into the target DNA. Thereby, the break in the DNA can be repaired, but random mutations of few base pairs can be generated, that can give rise to a mutant phenotype. Homology-directed repair (HDR), on the other hand, depends on a second donor DNA molecule that must be present in close proximity to the strand break of the target DNA. If this second DNA molecule has sufficient sequence homology to the region around the strand break, knock-in of sequences from the donor DNA into the target DNA through homologous recombination is possible. This allows generation of desired mutations or targeted replacement of DNA sequences, e.g. for gene correction.

The CRISPR nucleases known to date are divided into different types based on their mode of operation. They originate from different bacteria and/or archeae and differ in the size, domain structure, and the PAM-sequence recognized. Nevertheless, CRISPR/Cas nucleases depend on the basic principle of a RNA-guided nuclease activity. Cpf1 is an example of a CRISPR nuclease that differs from Cas9 in that it recognizes a different PAM-sequence and does not require a tracrRNA sequence in the gRNA (EP 3 009 511; Zetsche et al., Cell 163(3) (2015) 759-771). Cpf1, unlike Cas9, generates double strand breaks with sticky overhangs, facilitating introduction of new DNA sequences through ligation. Not only the discovery of new CRISPR systems and nucleases, but also the modification of known CRISPR nucleases can improve or extend the possibilities for sequence-specifically affecting target DNA. As mentioned above, a mutation of an amino acid essential for nuclease activity in Cas9 in one of the two subdomains responsible for DNA cleavage turns it into a nickase Cas9 (nCas9). This nCas9 cleaves only one strand of the double-stranded target DNA. Such nCas9 enzymes can be used to increase the specificity for double strand breaks in target DNA and reduce the number of unintended off-target cuts: when two nCas9 nucleases are applied simultaneously where one enzyme cut the coding strand and the other cuts the template strand in the target DNA, double strand breaks only occur when both nCas9 enzymes nick the DNA in the desired region.

In alternative ways of affecting target DNA, the entire nuclease activity of a CRISPR nuclease may be abolished by suitable mutations, which disarms the enzyme, resulting in a so-called “dead” CRISPR nuclease (e.g. dead Cas9 “dCas9”). The dead CRISPR nuclease can still interact with the gRNA to identify target DNA. A dead CRISPR nuclease may be fused to a protein domain with a desired function and thus the desired function may be targeted to the target DNA. A protein domain having such desired function may be an activator or repressor of gene transcription, whereby gene transcription at the target DNA may be affected. Alternatively, functions of such protein domains may affect epigenetic markers or may be used for genomic imaging with fluorescent protein probes.

It is to be expected that future research identifies further CRISPR systems and nucleases and that further modifications of target nucleic acids will be developed. However, the easy programming of CRISPR/Cas systems to identify and affect specific target nucleic acids through provision of a gRNA will remain the basic principle and may be combined with the present invention.

The picornaviruses used in the present invention such as TRSV have a rather broad host range. Therefore, gene editing according to the invention may be performed in many different plants. Notably, the experimental host range of TRSV is wide. Early publication of Price (American Journal of Botany 27 (1940) 530-541) reports about numerous species in 40 dicotyledonous and monocotyledonous families to be susceptible to TRSV. In nature, the virus occurs both in woody and in herbaceous plants. TRSV was reported to infect various plant species in Leguminoseae, Solanaceae, Chenopodiaceae, Compositeae, Cucurbitaceae, Scrophulariaceae etc. (Price 1940; Bulletin OEPP/EPPO Bulletin (2017) 47 (2), 135-145; R. Stacer-Smith (2014) Tobacco ringspot virus, accessible at http://wvvw.dpvweb.net/dpv/showdpv.php?dpvno=309).

Accordingly, the plant or cells thereof wherein gene editing according to the invention is carried is not particularly limited. The process can be applied to monocot and dicot plants, whereby the latter are preferred. The plant species for practicing this invention include, but not restricted to, representatives of Leguminoseae, Solanaceae, Chenopodiaceae, Compositeae, Cucurbitaceae, and Scrophulariaceae. Both crop and non-crop plants can be used. Common crop plants for the use in present invention include alfalfa, barley, beans, canola, cowpeas, cotton, corn, clover, lotus, lentils, lupine, millet, oats, peas, peanuts, rice, rye, sweet clover, sunflower, sweetpea, soybean, sorghum triticale, yarn beans, velvet beans, vetch, wheat, wisteria, and nut plants. Preferred plants are Glycine max (soybean). Phaseolus vulgaris, Lycopersicon esculentum, Vigna unguiculata, various Nicotiana species; Cucumis sativus; Nicotiana tabacum, N. clevelandii, Chenopodium amaranticolor and Vigna unguiculata. Particularly preferred plants are N. tabacum and N. benthamiana.

As described above, it is preferred that a plant or plant cell is provided with a DNA molecule encoding the RNA molecule of the invention, notably by Agrobacterium-mediated transfection. However, not all plant species are equally or similarly amenable to Agrobacterium-mediated transfection. Therefore, a plant well amenable to Agrobacterium-mediated transfection may be transfected by Agrobacterium-mediated transfection to generate and replicate the RNA molecule in said plant (“first plant”), collecting sap from the plant containing the RNA molecule of the invention and preferably also RNA1 of the picornavirus (such as TRSV), and using the sap for infecting a second plant to be transfected, e.g. by rubbing the sap on leaves of the second plant.

Accordingly, the invention provides a process of infecting a plant (“second plant”) such as soybean with a genetically-modified picornavirus, comprising
(A) providing, e.g. Agrobacterium-mediated, a first plant with the DNA molecule of the invention encoding the RNA molecule of the invention for expressing said RNA molecule in cells of said first plant, and
(B) infecting said plant to be transfected with said RNA molecule expressed in the first plant.

A preferred first plant is N. tabacum or N. benthamiana. Possible second plants are those mentioned above, such as Glycine max. Step (B) may comprise rubbing a leave of a first plant on a leave of the second plant optionally with abrasive, whereby cell sap of the first plant contacts the second plant such as a leave thereof. After step (A), it is also possible to collect cell sap from said first plant, said sap containing said RNA molecule. Step (B) may comprise rubbing or spraying said sap onto leaves, preferably using an abrasive. Regarding spray transformation using an abrasive, reference is made to WO2012019660.

Using the first plant or its cell sap, it is possible to infect two or more second plants. The second plants may be plants of the same line, species, or genera, or of different line, species, or genera. By infecting two or more different plants, e.g. of different line, species, or genera, following step (A), the infected plants may be screened for an altered phenotype due to the infection with the RNA molecule, whereby the overall process is very efficient.

Promoters

As used herein, the term “promoter active in plant cells” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the cauliflower mosaic virus 35S promoter (CaMV35S promoter) (Harpster et al. (1988) Mol Gen Genet. 212(1):182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters (such as the light-inducible Rubisco promoters), root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like. For transient expression, constitutive promoters, i.e. promoters that are not developmentally regulated, are preferably used. However, constitutive promoters may be tissue-specific or organ-specific. Preferred promoters are those used in the Examples described below.

### EXAMPLES

### Example 1

### Tobacco Ringspot Virus (TRSV) Constructs

cDNA copies of TRSV RNA1 (GenBank: KJ556849; SEQ ID NO: 1) and RNA2 (GenBank: KJ556850; SEQ ID NO: 2) as described by Zhao et al. (2016) were synthesized by Life Technologies and subcloned into binary vectors using modular cloning approach (Weber et al. 2008). Resulting construct pNMD36170 (SEQ ID NO: 3) contained RNA 1 insertion; construct pNMD36180 (SEQ ID NO: 4) encoded RNA2, both with a double 35S promoter and nos terminator (FIG. 1A). pNMD36180 construct was further modified by insertion of BsaI cloning site between CP and 3′UTR and ribozyme sequence between PolyA tail and nos terminator (resulting construct pNMD43050 (SEQ ID NO: 5, FIG. 1A).

The fragment of phytoene desaturase cDNA from Nicotiana benthamiana (NbPDS; GenBank: DQ469932.1; SEQ ID NO: 6; nucleotide position 520-734) was incorporated in direct orientation into pNMD43050 construct via BsaI cloning site, resulting in pNMD42330 vector (SEQ ID NO: 7).

pNMD43741 (SEQ ID NO: 8), the construct containing the fragment of phytoene desaturase cDNA from soybean (GmPDS; NM_001249840.2; SEQ ID NO: 9; nucleotide position 1374-1582) in direct orientation was created in a similar way (FIG. 1B).

The same cloning strategy was used to create RNA2 vectors carrying gRNAs:

1) pNMD45660 (SEQ ID NO: 10) containing the insertion of gRNA_GmFT2a_SP1 specific for Flowering Locus T 2a (FT2a) gene from soybean (Glyma16g26660; SEQ ID NO: 11; Cai et al., 2018). gRNA_GmFT2a_SP1 was composed of the target sequence (SEQ ID NO: 12; Cai et al., 2018) followed by gRNA scaffold sequence (SEQ ID NO: 13; Mali et al., 2013).

2) pNMD45680 (SEQ ID NO: 14) with gRNA_NbPDS_Hpa1 composed of the target sequence (SEQ ID NO: 15) and gRNA scaffold sequence (SEQ ID NO: 13; Mali et al., 2013). Target sequence was designed using CHOPCHOP (version 3) web tool (https://chopchop.cbu.uib.no) using the coding sequence of phytoene desaturase (PDS) gene from Nicotiana benthamiana (GenBank: EU165355.1; SEQ ID NO: 16) as a template.

3) pNMD46661 vector (SEQ ID NO: 17) with gRNA_NbPDS_Hpa1 and Arabidopsis U6 promoter (GenBank: CP002686.1; SEQ ID NO: 18).

4) pNMD47681 construct (SEQ ID NO: 19) with gRNA_Gm_D7_PDS18 specific for phytoene desaturase genes GmPDS11 (Glyma.11G253000; SEQ ID NO: 20) and GmPDS18 (Glyma.18G003900; SEQ ID NO: 21) from soybean (FIG. 1C). gRNA_Gm_D7_PDS18 is composed of target sequence (SEQ ID NO: 22; Du et al. 2015) and gRNA scaffold (SEQ ID NO: 13; Mali et al., 2013).

### Example 2

### Plasmid Vectors for the Stable Transformation of Nicotiana benthamiana and Soybean Plants

In case of pNMD27570 construct (SEQ ID NO: 23), for the selection on phosphinothricin, two expression cassettes were inserted between left and right borders of binary vector: 1) expression cassette for the selective gene comprising nos promoter, coding sequence of phosphinothricin N-acetyltransferase (BAR) and nos terminator; and 2) expression cassette for Cas9 endonuclease composed of 35S promoter, omega translational enhancer from Tobacco Mosaic Virus, coding sequence of Cas9 endonuclease protein from Streptococcus pyogenes (GenBank: AKQ21048.1) codon-optimized for Arabidopsis (SEQ ID NO: 24) and octopin synthase (ocs) terminator (FIG. 2, top).

Construct pNMD34661 (SEQ ID NO: 25) for the selection on hygromycin had same Cas9 expression cassette and hygromycin transferase (HPT) expression cassette composed of nos promoter, omega translational enhancer, HPT coding sequence and nos terminator (FIG. 2, bottom).

### Example 3

### Stable Transformation of Nicotiana benthamiana and Soybean Plants with Binary Vectors for the Expression of Cas9 Gene

Stable transgenic Nicotiana benthamiana plants expressing Cas9 protein were produced by Agrobacterium-mediated genetic transformation (GV3101 strain, plasmid construct pNMD27570, FIG. 2) using a standard protocol (Horsch et al. 1985).

Transgenic plants of soybean ‘Fayette’ expressing Cas9 protein were produced by cotyledonary-node method (Olhoft et al., 2003 with slight modifications) using EHA105 strain of Agrobacterium tumafaciens carrying pNMD34661 construct (FIG. 2). Transgenic clones were multiplied on Shoot Elongation Medium (SEM) supplemented by 10 mg/l hygromycin and solidified with 8 g/l of agar (Duchefa Biochemie B.V., Haarlem, The Netherlands or Sigma-Aldrich, St. Louis, Mo., USA). All shoot cultures were dried 30-40 min under the hood after transferred to the fresh SEM media. Sometimes stronger shoots were dried 2-5 days in empty Petri dishes in growth room and then placed onto fresh SEM medium. Both approaches greatly reduced shoot vitrification and improved rooting. Shoots 1.5-2 cm long were cut and rooted on Rooting Medium (MS half strength medium containing 20 g/l sucrose and 0.5-1.0 mg/l indole-3-acetic-acid (IAA) and solidified with 6 g/l agar). Plants obtained could be ether multiplied by cutting or established in the soil.

### Example 4

### Agrobacterium-Mediated Transfection of Nicotiana benthamiana Plants with TRSV Constructs

33 days-old Nicotiana benthamiana wild type plants were infiltrated with mixtures of Agrobacterium tumefaciens cultures (strain ICF320) harboring TRSV-based vectors. For infiltration, OD600 of overnight cultures was adjusted to 1.5 and further diluted 1:100 with infiltration buffer containing 10 mM MES (pH 5.5) and 10 mM MgSO4. Two leaves for each plant were infiltrated using the needleless syringe with a mixture of two cultures: 1) pNMD36170 and pNMD36180 for TRSV wild type virus; and 2) pNMD36170 and pNMD42330 for TRSV with an insertion of Nicotiana benthamiana PDS cDNA fragment in sense orientation.

Plant phenotype was evaluated 17 days post infiltration (dpi). In contrast to untreated Nicotiana benthamiana (negative control, FIG. 3A), plants inoculated with wild type TRSV constructs demonstrated light chlorosis typical for TRSV infection (FIG. 3B). Plants inoculated with PDS silencing constructs had clear photobleaching phenotype characteristic for PDS gene silencing (FIG. 3C). These results prove successful infection of Nicotiana benthamiana plants with TRSV using agrobacterial delivery.

### Example 5

### Mechanical Inoculation of Nicotiana benthamiana Plants with TRSV Viral Particles

17 dpi, we extracted the sap of TRSV-infected plants (Example 4) using 10 mM sodium phosphate buffer, pH 7.0. To avoid the contamination with Agrobacterium, only systemic non-infiltrated leaves were harvested for this purpose. Sap aliquots were plated on agar media with selective antibiotics; no Agrobacteria were detected.

This sap was used for the mechanical inoculation of another 32 days-old Nicotiana benthamiana plants. For this purpose, plant sap preparations mixed with silicon carbide F800 particles (Mineraliengrosshandel Hausen GmbH, Telfs, Austria) were gently rubbed onto the surface of leaves in such a way as to break the surface cells without causing too much mechanical damage. For each plant, two leaves were rubbed.

14 days post rubbing (dpr), plants showed distinct phenotypes: characteristic light chlorosis in case the infection with TRSV wild type virus (FIG. 4A) and intensive leaf photobleaching due to PDS silencing in case of TRSV viral particles with a fragment of PDS gene (FIG. 4B). These data indicate successful mechanical inoculation of Nicotiana benthamiana plants with TRSV viral particles which are present in the sap of infected plants.

### Example 5

### Mechanical Inoculation of Soybean Plants with TRSV Viral Particles

Our experiments on Agrobacterium-mediated inoculation of soybean plants with TRSV failed. That is why we focused on mechanical inoculation using the sap from TRSV infected Nicotiana benthamiana plants.

Leaves of 17-days-old soybean plants ‘Bliskavitsa’ were rubbed with a sap from leaves of TRSV-infected Nicotiana benthamiana plants extracted at 16 dpi. The procedure was performed as described in Example 4. For each plant, three leaves were rubbed.

In soybean, the results of mechanical inoculation were clearly visible already at 14 dpr. All the plants rubbed with viral particles were infected. At a closer look, the empty vector showed typical viral phenotype with a light chlorosis of young leaves (FIG. 5B), compare to untreated plants (FIG. 5A). Remarkably, constructs with a fragment of GmPDS had a strong photobleaching phenotype (FIG. 5C).

We also analyzed the presence of viral RNA in soybean plants mechanically inoculated with TRSV particles. To obtain viral particles, we first inoculated Nicotiana benthamiana plants with Agrobacterium strain ICF320 carrying RNA2 constructs for empty vector (pNMD43050), GmFT2a_SP1 gRNA (pNMD45660) and soybean PDS fragment in sense orientation (TRSV-GmPDSfragm, pNMD43741) in combination with the RNA1 construct (pNMD36171) using the infiltration with needle-less syringe. Plant sap was extracted from TRSV-infected and control plants 20 days post infiltration as described above. Three soybean ‘Bliskavitsa’ plants (23 days old, primary leaves and first trifoliate) were rubbed with silicon carbide F800 and virus particle-containing N. benthamiana sap.

21 days post rubbing, leaf samples were collected and used for isolation of total RNA. RNA isolation was performed using Nucleospin RNA Plant Kit (Macherey-Nagel GmbH & Co. KG, Duren, Germany) according to manufacturer's protocol. cDNA synthesis was carried out using PrimeScript RT Reagent Kit (Takara Bio Europe, Saint-Germain-en-Laye, France) with 500 ng RNA and oligo(dT) priming for 30 min 37° C. For the POR, TRSV-specific primers flanking insertion of gRNA or silencing fragment were used: trsv-sil-fwd (TCAATGCTAAGGACATAGTTGCAC, SEQ ID NO: 41) and trsv-sil-rev (TATTGACGCTTCTATCTAACCAACC, SEQ ID NO: 42). The results of RT-PCR analysis are shown in FIG. 6. TRSV-specific amplificates of the expected size were obtained for all samples except the control plants rubbed with extract from uninfected N. benthamiana), whereas no amplification was detected if no reverse transcriptase (RT) was added to the reaction showing that the signals are not due to DNA contamination. In plant/replicate 1 from the infection with the TRSV carrying the PDS silencing fragment, a shorter PCR fragment was detected, meaning instability of the RNA2. Therefore, we could demonstrate that TRSV can be used for systemic expression of gRNAs in soybean.

### Example 6

### TRSV-Mediated Editing in Cas9-Transgenic N. benthamiana

Experiment I. To analyze if the Cas9 protein accepts guide RNAs in the context of the TRSV RNA2, 4 weeks old Cas9-transgenic N. benthamiana plants transformed with pNMD27570 construct (3 plants each) were inoculated with ICF320 Agrobacteria delivering empty TRSV (pNMD36170 and pNMD43050 constructs), TRSV vector with NbPDS_Hpa1 gRNA insertion (pNMD36170 and pNMD45680) and TRSV vector containing the insertion of NbPDS_Hpa1 gRNA with the additional Arabidopsis U6 promoter upstream of gRNA (pNMD36170 and pNMD46661) (FIG. 1C). Furthermore, wild type N. benthamiana plants were inoculated with TRSV-gRNAs vectors as negative control.

Leaf samples for editing analysis were taken 16 and 24 days post infiltration. TRSV symptoms were detectable for the NbPDS_Hpa1 gRNA without additional promoter on three out of three Cas9-transgenic plants (and one wild type N. benthamiana) and for the TRSV empty control on two out of two plants, but for the construct with the AtU6 promoter in front of the gRNA no symptoms on three out of three Cas9-transgenic plants (and one wild type N. benthamiana) each were visible. Further RT-PCR analysis revealed that the additional promoter sequence in the TRSV construct is destabilizing for the virus.

To analyze editing events, genomic DNA was isolated using NucleoSpin Plant II Kit (Macherey-Nagel GmbH & Co. KG, Duren, Germany) according to manufacturer's instructions, and the PDS target sequence was analyzed regarding possible sequence changes. 150 ng genomic DNA were treated with Hpal enzyme to digest PDS wild type sequence. The following PCR was performed with target-specific oligonucleotides PDS_in2_fwd (GTGTGATGCTGGATTTATGATCGTGG, SEQ ID NO: 33) and PDS_in2_rev (CTAGCTTATGAGATGAGACCAAGGACCTC, SEQ ID NO: 34) using undigested and digested gDNA as a template. Nicotiana benthamiana has two PDS genes: NbPDS3a (Niben101Scf01283g02002.1; SEQ ID No: 26) and NbPDS3b (Niben101Scf14708g00023.1; SEQ ID No: 27). PDS_in2_fwd and PDS_in2_rev oligos were designed so that they preferentially amplified NbPDS3a template. FIG. 7 shows the result of PCR analysis for 16 dpi plant material. If undigested gDNA was used as template, a PCR product with the expected size was amplified in all samples (FIG. 7, bottom panel). Hpal treatment led to differences in the amplification pattern: stronger signals were detected when the Cas9-transgenic plants were treated with TRSV-gRNA constructs in comparison to the control samples and the construct with the additional promoter, implying CRISPR/Cas9-mediated editing took place (FIG. 7, top panel).

PCR products resulting from PCR with digested gDNA as a template from the TRSV NbPDS_Hpa1 gRNA or empty control infected plants were cloned into pJET1.2 vector (Thermo Fisher Scientific, Waltham, Mass., USA), and individual clones were analyzed by sequencing to detect editing events. As a reference sequence (Reference Sequence 1), we used 59 nucleotide long fragment of Nicotiana benthamiana phytoene desaturase gene NbPDS3a (Niben101Scf01283g02002.1; SEQ ID NO: 29), nucleotide position 1201-1259) comprising the gRNA target in the position 20-39 nt and flanking sequences (Reference sequence 1; SEQ ID NO: 28; FIG. 8).

Table 1 summarizes sequence analysis for Experiment I. In total, we analyzed 34 sequences. All negative controls (wild type plant inoculated with TRSV-gRNA_NbPDS_Hpa1 construct, uninfected Cas9 transgenic plant and Cas9 transgenic plant infected with an empty virus) did not show any modifications: all 59 nucleotides matched with a Reference Sequence (sequences pattern 59M). Editing were found in 19 out of 25 sequences (75%) from Cas9-transgenic plants infected with TRSV NbPDS_Hpa1 gRNA. All detected editing events were deletions of 1, 4, 5, 6, 8, 9, and 11 nucleotides. Sequence patterns observed are summarized in Table 1. For example, sequence pattern “32M-6D-21M” reads as “first 32 nucleotides match with a Reference Sequence, next 6 nucleotides are deleted, last 21 nucleotides match with a Reference Sequence”. Therefore, we could show that Cas9 protein accepts guide RNAs in the context of the TRSV RNA2 and mediates editing.

TABLE 1
TRSV-mediated editing in Cas9-transgenic N. benthamiana
(Experiment I): sequence patterns of individual clones.

| | | | | Total |
|---|---|---|---|---|
| | | Sequence | Sequences | number of |
| Plant | Construct | pattern | with pattern | sequences |
| Wild type | TRSV- | 59M | 2 | 2 |
| | gRNA_NbPDS_Hpa1 | | | |
| Cas9 | No virus | 59M | 3 | 3 |
| transgenic | TRSV empty | 59M | 4 | 4 |
| | TRSV- | 59M | 6 | 25 |
| | gRNA NbPDS Hpa1 | 38M-1D-20M | 2 | |
| | | 33M-4D-22M | 2 | |
| | | 34M-4D-21M | 1 | |
| | | 34M-5D-20M | 3 | |
| | | 33M-5D-21M | 1 | |
| | | 32M-6D-21M | 1 | |
| | | 33M-6D-20M | 4 | |
| | | 31M-8D-20M | 2 | |
| | | 31M-9D-19M | 2 | |
| | | 28M-11D-20M | 1 | |

M: nucleotides matching with Reference Sequence;
D: deleted nucleotides

To confirm these findings, we repeated the experiment with little modifications.

Experiment II. Eight Cas9-transgenic N. benthamiana plants were inoculated with Agrobacteria delivering TRSV empty control (pNMD43055), NbPDS_Hpa1 gRNA gRNA (pNMD45681) or TRSV GmFT2a_SP1 gRNA (pNMD45661), as a negative control. Samples for editing analysis were taken after 28 days. 11 and 14 days after inoculation TRSV symptoms were visible on all infected plant, but no PDS silencing was detectable to this timepoint. After 28 days, TRSV symptoms were still visible on all plants; some leaf bleaching was clearly visible on all eight plants inoculated with Agrobacteria delivering TRSV NbPDS_Hpa1 gRNA (pNMD45681).

Editing analysis was performed as described above for Experiment I. PCR products amplified on digested genomic DNA were cloned into pJET1.2 vector, and single clones were analyzed by sequencing to detect editing events. For TRSV-gRNA_NbPDS_Hpa1 plant material, one could see numerous editing events, mainly deletions, in two cases insertions. In total, 20 out 22 clones (91%) from TRSV-gRNA_NbPDS_Hpal infected plants were edited. All 9 NbPDS sequences amplified from TRSV-gRNA_GmFT2a_SP1 plant material contained no edits. Sequencing data are summarized in Table 2.

TABLE 2
TRSV-mediated editing in Cas9-transgenic N. benthamiana
(Experiment II): sequence patterns of individual clones.

| | | | | Total |
|---|---|---|---|---|
| | | Sequence | Sequences | number of |
| Plant | Construct | pattern | with pattern | sequences |
| Cas9 | TRSV- | 59M | 9 | 9 |
| transgenic | gRNA_GmFT2a_SP1 | | | |
| | TRSV- | 59M | 2 | 22 |
| | gRNA_NbPDS_Hpa1 | 39M-1D-19M | 1 | |
| | | 36M-4D-19M | 2 | |
| | | 34M-5D-20M | 1 | |
| | | 33M-6D-20M | 3 | |
| | | 32M-7D-20M | 2 | |
| | | 32M-8D-19M | 1 | |
| | | 30M-9D-20M | 3 | |
| | | 27M-10D-22M | 1 | |
| | | 28M-10D-21M | 1 | |
| | | 29M-14D-14M | 1 | |
| | | 5M-49D-5M | 2 | |
| | | 40M-1I-19M | 2 | |

M: nucleotides matching with Reference Sequence;
D: deleted nucleotides

### Example 7

### Deep Sequencing Analysis for the Evaluation of TRSV-Mediated Editing of PDS Genes in Cas9-Transgenic N. benthamiana

Cas9 transgenic Nicotiana benthamiana plant #1 from the inoculation with TRSV-gRNA_NbPDS_Hpa1 (pNMD45681) and Cas9 transgenic Nicotiana benthamiana plant #4 TRSV inoculated with TRSV-gRNA_GmFT2a_SP1 (pNMD45661) were selected for deep sequencing analysis. Reference Sequences of phytoene desaturase genes from Nicotiana benthamiana are shown in FIG. 9. This plant species has two genes coding for PDS: NbPDS3a (Niben101Scf01283g02002.1; Ref2; SEQ ID NO: 29) and NbPDS3b (Niben101Scf14708g00023.1; Ref3; SEQ ID NO: 30) (FIG. 9A). Reference sequence used for deep sequencing analysis consists of 79 nucleotides. NbPDS3a and NbPDS3b have 4 mismatches in nucleotide positions 1, 42, 52 and 76. gRNA target sequence (nucleotide position 7-26) is identical for both NbPDS3a and NbPDS3b (FIG. 9B).

100 ng genomic DNA from 28 dpi plant material were used for first PCR with Phusion High-Fidelity DNA Polymerase polymerase (Thermo Fisher Scientific, Waltham, Mass., USA) introducing adaptor sequences and wobble bases needed for second PCR and sequencing, which was done afterwards at Microsynth Seqlab (Gottingen, Germany). For PCR, we used next oligos: PDS_ex3_wob_fwd (TCGTCGGCAGCGTCAGATGTGTATAAGAGACAGNNNNNNNNNNGTAGTCTGCATTGA TTATCCAAGACC, SEQ ID NO: 35) and PDS_ex3_wob_rev (GTCTCGTGGGCTCGGAGATGTGTATAAGAGACAGNNNNNNNNNNCTGCACCAGCAAT AACAATCTCCAATGG, SEQ ID NO: 36). This oligo pair amplified fragments of both NbPDS3a and NbPDS3b genes.

The first PCR was carried out once with 20 cycles and once with 35 cycles, from which the product was loaded on a 2% agarose gel. The product of the correct size was cut out of the gel and extracted. Both “variants” (low cycle number vs. gel extraction) were used for the second PCR. Separation of the second PCR products via capillary electrophoresis (quality control) showed several fragments in the “low cycle number” samples. In the samples with the gel-extracted templates for the second PCR showed only one peak and were therefore selected for the deep sequencing.

Deep sequencing resulted in 38.5 Mio reads for the Nb_PDS_Hpa1 sample and 33 Mio reads for the control gRNA GmFT2a_SP1 sample (Table 3).

TABLE 3
Statistics of deep sequencing performance.

| Sample | Reads | Bases |
|---|---|---|
| TRSV-gRNA_NbPDS_Hpal in | 38,585,335 | 3,007,611,150 |
| Cas9-transgenic Nicotiana benthamiana | | |
| TRSV-gRNA_GmFT2a_SP1 in | 32,967,764 | 2,603,471,505 |
| Cas9-transgenic Nicotiana benthamiana | | |

Sequencing data were subjected to InDel analysis, meaning the reads was analyzed for insertions and deletions, but not for substitutions, and assigned to a respective pattern. The reads of both samples were trimmed (cutting of adaptor, wobble and primer sequences) and aligned to 79 nt of Reference 2 (NbPDS3a) and separately to Reference 3 (NbPDS3b). Identical sequences were summarized in one cluster with the respective size (number of reads) (FIG. 10). The cluster with the biggest size (49.7%) corresponds to the wild type PDS fragment (79M, 79 match), the second cluster (26.15%) to single nucleotide deletion in the gRNA target region (24M-1D-54M), and the third (7.2%) to a single nucleotide insertion in the gRNA target region (24M-1I-55M). With lower frequencies deletions in the gRNA target region from two to eleven nucleotides were found and another one nucleotide insertion. Only two clusters with lower frequencies (0.15% and 0.11%) with deletions out of the target region are found.

The summary of sequence analysis for control gRNA_GmFT2a_SP1 gRNA sample is shown in FIG. 11. The cluster with the by far biggest size (99.05%) corresponds to the wild type PDS fragment (79M, 79 match), and mutations in the other clusters are mainly outside of the target region, showing that the mutations observed in the gRNA_Nb_PDS_Hpa1 sample are due to the presence of the specific gRNA. The Indel analysis with the alignment of the reads to Reference 3 (NbPDS3b) gave exactly the same results in comparison to the alignment with Reference 2 (NbPDS3a) due to the fact that substitutions are ignored in this analysis, and is therefore not separately shown. Both genes differ in the 79 nt fragment only in four nucleotides (1=A1G; 2=T42C; 3=G52A; 4=T76A).

In summary, the deep sequencing analysis showed that in the PDS gRNA sample only 50% of the PDS sequences have no insertions or deletions (but some of them might have substitutions in the target region, frequencies were not calculated), the main modifications are a single nucleotide deletion or insertion close to the PAM and both PDS genes are targeted to the same extent, but there are no editing-induced recombinations between two genes observed.

### Example 8

### TRSV-Mediated Gene Editing in Soybean Shoots Culture

Experiment I. Cas9 transgenic soybean ‘Fayette’ N1 shoots (pNMD34661, Example 2) were used for direct inoculation with TRSV viral particles in vitro. TRSV particles were generated in N. benthamiana. For this purpose, N. benthamiana plants were syringe infiltrated with Agrobacteria delivering TRSV constructs with GmFT2a_SP1 (pNMD45661) and Gm_D7_PDS18 (pNMD47681) gRNAs (FIG. 10) in combination with RNA1 construct pNMD36170 (FIG. 1A). Gm_D7_PDS18 gRNA was designed as described in Du et al. (2015). N. benthamiana leaves were harvested 27 days post inoculation. Leaf material was ground with 4 fold volume of 10 mM phosphate buffer (pH 7.0), filtered through Miracloth, centrifuged for 15 min at 5500 rpm, and sterilized by filtration through 20 μm bottle top filter.

Soybean shoots were sprayed with sterile plant sap containing viral particles and supplemented with 1.5% silicon carbide F800 using manual sprayer. Sprayed shoots were transferred directly after spraying to fresh SEM medium and cultivated at 25° C. in the light.

9 weeks post spraying, soybean shoots (FIG. 12) were analyzed for editing events. For editing analysis, genomic DNA of the ‘Fayette’ shoots was isolated using NucleoSpin plant II kit (Macherey-Nagel GmbH & Co. KG, Duren, Germany). 100 ng genomic DNA was treated with BfaI restriction enzyme to cleave the wild type sequence. Following PCR was performed with target-specific oligonucleotides with undigested and digested gDNA as template. Soybean contains two PDS genes, GmPDS11 (Glyma.11G253000; SEQ ID NO: 20) and GmPDS18 (Glyma.18G003900; SEQ ID NO: 21). Gm_D7_PDS18 gRNA target sequence (SEQ ID NO: 22) is common for both genes, without any mismatch. PCR primers were designed so that they should amplify separately either GmPDS11 or GmPDS18.

### D7-PDS18_11_PCR_f (AAGGTATCCTGATATCATGTTGG, SEQ ID NO: 37) and

D7-PDS18_11_PCR_r (AATCTTCAACAAGCCTGATGATG, SEQ ID NO: 38) were specific for GmPDS11.

### D7-PDS18_18_PCR_f (TTATGGAATTTTAGGTACCCTG, SEQ ID NO: 39) and

D7-PDS18_18_PCR_r (TGATGAAGTTATAAAGCCAACG, SEQ ID NO: 40) were specific for GmPDS18. Both oligo pairs amplified PCR fragment containing BfaI restriction site. GmPDS11-specific oligos amplify 373 bp fragment of GmPDS11, and GmPDS18-specific oligos amplify 356 bp fragment of GmPDS18. These fragments share 84.5% identity on the nucleotide level.

PCR products were cloned into pJET1.2 vector, and individual clones were sequenced. For sequences analysis, Reference Sequence 4 (Ref4, SEQ ID NO: 31, GmPDS11 fragment) and Reference Sequence 5 (Ref5, SEQ ID NO: 32, GmPDS18 fragment) were used. Results of sequence analysis are summarized in Table 4.

TABLE 4
TRSV-mediated editing in Cas9-transgenic soybean shoots
(Experiment I): sequence patterns of individual clones.

| | | GmPDS | | | Total |
|---|---|---|---|---|---|
| | Primer | homolog | Sequence | Sequences | number of |
| Construct | specificity | sequence | pattern | with pattern | sequences |
| TRSV- | GmPDS11 | GmPDS11 | 59M | 18 | 22 |
| gRNA_GmFT2_SP1 | | GmPDS18 | 59M | 4 | |
| | GmPDS18 | GmPDS11 | 59M | 1 | 20 |
| | | GmPDS18 | 59M | 19 | |
| TRSV- | GmPDS11 | GmPDS11 | 59M | 11 | 15 |
| gRNA_D7_PDS18 | | | 43M-2D-40M | 1 | |
| | | | 37M-8D-40M | 2 | |
| | | | 45M-1I-40M | 1 | |
| | | GmPDS18 | 59M | 2 | 5 |
| | | | 42M-4D-39M | 1 | |
| | | | 39M-6D-40M | 1 | |
| | | | 40M-6D-39M | 1 | |
| | GmPDS18 | GmPDS11 | 59M | 1 | 1 |
| | | GmPDS18 | 59M | 3 | 18 |
| | | | 43M-1D-42M | 1 | |
| | | | 43M-2D-40M | 2 | |
| | | | 42M-3D-40M | 3 | |
| | | | 38M-6D-41M | 2 | |
| | | | 40M-6D-39M | 1 | |
| | | | 38M-7D-40M | 1 | |
| | | | 35M-9D-41M | 1 | |
| | | | 37M-9D-39M | 2 | |
| | | | 35M-10D-40M | 1 | |
| | | | 35M-11D-39M | 1 | |

M: nucleotides matching with Reference Sequence;
D: deleted nucleotides;
I: inserted nucleotides

GmPDS11 sequence pool contained the minor fraction of GmPDS18 sequences and vice versa indicating certain level of cross-amplification (Table 4). When GmPDS11-specific primers were used for PCR, sequencing showed no modification in 18 GmPDS11 and 4 GmPDS18 sequences in the GmFT2a_SP1 gRNA control sample. In contrast, in the D7_PDS18 gRNA sample, 3 out of 15 GmPDS11 sequences showed deletions in the gRNA target region (Table 4). All 5 GmPDS18 sequences from this sample contained edits: 3 sequences were with deletions and 2 with insertions (Table 4).

When GmPDS18-specific oligos were used for PCR, sequencing showed no modification of 19 GmPDS18 (Table 4) and 1 GmPDS11 (Table 4) sequences in the GmFT2a_SP1 gRNA control sample. In the D7_PDS18 gRNA sample, 15 out of 18 GmPDS18 sequences showed deletions in the gRNA target region (Table 4); one GmPDS11 sequence found in this sample had no modification (Table 4).

Sequencing data for Experiment I are summarized in Table 5. In total, we found 60% edited GmPDS sequences, and separate examination of PDS homologs shows that GmPDS18 is more efficiently edited than GmPDS11 (87% vs 24%), although both genes have identical target sequence.

TABLE 5
Summary of soybean PDS sequence analysis after TRSV-mediated
gene editing 9 weeks post spraying (Experiment I, Example 8).

| | Sequence pattern | | | | |
|---|---|---|---|---|---|
| | | | Wild | Total | % |
| Sequence | Deletion | Insertion | type | number | edited |

gRNA_D7-PDS18 sample

| | | | | | |
|---|---|---|---|---|---|
| GmPDS11 | 3 | 1 | 13 | 17 | 23.5 |
| GmPDS18 | 18 | 2 | 3 | 23 | 87.0 |

gRNA_GmFT2a_SP1 sample

| | | | | | |
|---|---|---|---|---|---|
| GmPDS11 | 0 | 0 | 18 | 18 | 0 |
| Gm PDS 18 | 0 | 0 | 23 | 23 | 0 |

Experiment II. The infection of the Cas9-transgenic ‘Fayette’ N1 (pNMD34661, Example 2) shoots was repeated with a higher number of shoots (4) per construct to have the possibility to analyze more different timepoints. Therefore, again TRSV particles were generated/multiplied in N. benthamiana. Six N. benthamiana plants each were inoculated with Agrobacteria delivering constructs for expression of the GmFT2a_SP1 (pNMD45660) and Gm_D7_PDS18 (pNMD47681) gRNAs. Plant sap extraction and the treatment of the shoots with viral particles was performed as described above for the Experiment I. Directly after spraying shoots were transferred to fresh SEM medium with antibiotics and cultivated at 25° C. in the light.

Phenotypes of soybean shoots 14 days post spraying are shown in FIG. 14. There is no difference in growth and shape of the shoots visible if they were treated with TRSV extracts in comparison to control plant extracts from uninfected N. benthamiana plants. One shoot per treatment was harvested and was analyzed for PDS gene editing, the other three shoots were transferred to fresh SEM medium (in Magenta boxes) and were incubated further. Editing analysis was performed as described for Experiment I.

The D7_PDS18 gRNA can target both, GmPDS11 and GmPDS18 (without mismatch). For the PCR after the digestion with BfaI again the two primer combinations were used: one for amplification of GmPDS11 (D7-PDS18_11_PCR_f/r) and one primer combination for amplification of GmPDS18 (D7-PDS18_18_PCR_f/r). When PDS11-specific primers were used for the amplification of PDS from the control sample (infection with TRSV-gRNA_GmFT2a_SP1 (pNMD45661)), sequencing identified 24 GmPDS11 and 1 GmPDS18 sequences with no modification, showing the specificity D7_PDS18 gRNA (Table 6).

TABLE 6
TRSV-mediated editing in Cas9-transgenic soybean shoots
(Experiment II): sequence patterns of individual clones.

| | | | | | Total |
|---|---|---|---|---|---|
| | Primer | GmPDS | Sequence | Sequences | number of |
| Construct | specificity | homolog | pattern | with pattern | sequences |
| TRSV- | GmPDS11 | GmPDS11 | 59M | 24 | 24 |
| gRNA_GmFT2_SP1 | | GmPDS18 | 59M | 1 | 1 |
| | GmPDS18 | GmPDS11 | 59M | 2 | 2 |
| | | GmPDS18 | 59M | 23 | 23 |
| TRSV- | GmPDS11 | GmPDS11 | 59M | 16 | 18 |
| gRNA_D7_PDS18 | | | 43M-2D-40M | 1 | |
| | | | 43M-2D-40M | 1 | |
| | | | 46M-1I-39M | 1 | |
| | | GmPDS18 | 59M | 0 | 8 |
| | | | 41M-3D-41M | 1 | |
| | | | 40M-5D-40M | 1 | |
| | | | 39M-6D-40M | 2 | |
| | | | 37M-7D-31M | 1 | |
| | | | 38M-7D-40M | 2 | |
| | | | 45M-1I-40M | 1 | |
| | GmPDS18 | GmPDS11 | 59M | 2 | 2 |
| | | GmPDS18 | 59M | 10 | 25 |
| | | | 41M-3D-41M | 5 | |
| | | | 41M-4D-40M | 1 | |
| | | | 39M-5D-41M | 1 | |
| | | | 40M-5D-40M | 1 | |
| | | | 38M-6D-41M | 1 | |
| | | | 36M-9D-40M | 1 | |
| | | | 31M-13D-41M | 1 | |
| | | | 45M-1I-40M | 4 | |

M: nucleotides matching with Reference Sequence;
D: deleted nucleotides;
I: inserted nucleotides

Using PDS11 primers for the TRSV D7_PDS18 gRNA sample, we identified 18 GmPDS11 sequences out of which two showed a deletion and one a single nucleotide insertion in the gRNA target site (Table 6). In this sample, we also found 8 GmPDS18 sequences out of which seven showed a deletion and one a single nucleotide insertion in the gRNA target site (Table 6).

Using PDS18 primers for the amplification of PDS from the control sample (infection with TRSV GmFT2a_SP1 gRNA (pNMD45661)), we identified 23 GmPDS18 and 2 GmPDS11 sequences with no modification (Table 6). Using PDS18 primers for the TRSV D7_PDS18 gRNA sample, we identified 25 GmPDS18 sequences out of which 11 showed a deletion and 4 a single nucleotide insertion in the gRNA target site, and two PDS11 sequences with no modifications (Table 6).

A summary of the editing analysis in Cas9-transgenic ‘Fayette’ shoots two weeks after spraying (Experiment II) is shown in Table 7. Non-specific gRNA_GmFT2a_SP1 was not able to mediate editing in the Cas9-transgenic Fayette shoots, but the functionality of the D7_PDS18 gRNA could be reproduced, and it was even more efficient compared to the Experiment I. We found 48% edited PDS sequences already 14 days after spraying, and separate examination of PDS homologs shows that GmPDS18 is more efficiently edited by the D7_PDS18 gRNA than GmPDS11 (70% vs 14%).

TABLE 7
Summary of soybean PDS sequence analysis after TRSV-mediated gene
editing 2 weeks post spraying (Experiment II, Example 8).

| | Sequence pattern | | | | |
|---|---|---|---|---|---|
| | | | | Total | |
| Sequence | Deletion | Insertion | Wild type | number | % edited |

gRNA_D7-PDS18 sample

| | | | | | |
|---|---|---|---|---|---|
| GmPDS11 | 2 | 1 | 18 | 21 | 14.3 |
| GmPDS18 | 18 | 5 | 10 | 33 | 69.7 |

gRNA_GmFT2a_SP1 sample

| | | | | | |
|---|---|---|---|---|---|
| GmPDS11 | 0 | 0 | 23 | 26 | 0 |
| GmPDS18 | 0 | 0 | 23 | 24 | 0 |

### Example 9

### TRSV-Mediated Editing in Cas9-Transgenic Nicotiana benthamiana Analysis of F1 Generation

Cas9-transgenic Nicotiana benthamiana plants transformed with pNMD27570 construct were inoculated with ICF320 Agrobacteria delivering empty TRSV (pNMD43050 construct) and TRSV vector with NbPDS_Hpa1 gRNA insertion (pNMD45680 construct) as described in Example 6. These plants (F0 generation) were further incubated in the greenhouse, and seeds were harvested for analysis of the next (F1) generation.

The seeds were sown on plates with agarized M400 medium (half concentration of Murashige & Skoog salts and vitamins (Duchefa) and 15 g/l sucrose) supplemented with 5 mg/l phosphinothricin for selection of Cas9 transgenics. After one month, 47 seedlings of F1 generation obtained from plant #1 and plant #2 of F0 generation (Cas9-transgenic N. benthamiana inoculated with TRSV Nb_Hpa1 gRNA) were transferred to Magenta boxes, and 3 weeks later to the soil. AU other seedlings were pooled and analyzed for a presence of TRSV viral RNA using RT-PCR as described in Example 5, and for PDS gene editing events as described in Example 6. An overview of PDS sequence analysis in pooled seedling samples is represented in Table 8. TRSV-specific RT-PCR amplification products were detected in all pools of F1 seedlings obtained from F0 plants infected by TRSV, showing that the virus is transferred to the next generation. Editing analysis revealed that all 17 sequences from the pool of seedlings infected with TRSV empty vector matched to the wildtype PDS sequence, whereas PDS gene editing events were detectable in TRSV NbPDS_Hpa1 gRNA seedlings pool obtained from plant #1 (8 out of 17 edited) and plant #2 (7 out of 18 edited).

Based on these results, one can assume that either CRISPR/Cas9-mediated gene editing events produced in F0 generation are inherited in F1 generation or the editing process is still ongoing in this generation because of the transmitted TRSV.

TABLE 8
Summary of Nicotiana benthamiana PDS sequence analysis in F1 generation
seedlings after TRSV-mediated gene editing (pooled samples).

| | F0 | Number of | Sequence pattern | | | | |
|---|---|---|---|---|---|---|---|
| | plant | F2 | | | Wild | Total | % |
| Construct | line | seedlings | Deletion | Insertion | type | number | edited |
| TRSV empty | #1 | 134 | 0 | 0 | 7 | 7 | 0 |
| | #2 | 298 | 0 | 0 | 10 | 10 | 0 |
| TRSV- | #1 | 178 | 6 | 2 | 9 | 17 | 47 |
| gRNA_NbPDS_Hpa1 | #2 | 42 | 7 | 0 | 11 | 18 | 61 |

Next, individual mature plants of the F1 generation (Cas9-transgenic N. benthamiana inoculated with TRSV-Nb_Hpa1_gRNA) were analyzed for the presence of TRSV RNA using RT-PCR. Leaf material harvested 14 days after the plants had been transferred from the Magenta boxes to the soil was used for analysis. TRSV RNA was detectable in 35 out of 47 F1 plants, but only in three of them (#4-1-3, #4-2-2 and #4-3-1) PCR fragment had the expected size (Table 9); in the others a smaller fragment was amplified, implying instability of the virus and loss of the guide RNA sequence (Table 9). For 12 out of 47 plants, no TRSV was detected anymore (in contrast to the seedlings pools).

To dissect if these PDS gene editing events appear de novo in F1 generation because of the presence of TRSV, or editing events produced in F0 generation are inherited in F1 generation independent of the presence of TRSV, samples from 12 plants were selected for editing analysis (3 plants with stable TRSV containing gRNA insert, 4 plants with instability of TRSV (gRNA sequence eliminated), and 5 plant with no detectable TRSV at all. 17 days after transfer to the soil, leaf material of the indicated plants was harvested, and PDS gene editing events were analyzed as described in Example 6. Table 9 shows summarized RT-PCR data and PDS gene editing analysis. In case of stable TRSV vector, we have found editing events in two plants (#4-1-3 and #4-2-2) and no editing events in one plant (#4-3-1). In case of instable TRSV with gRNA sequence deletion, no editing was detected (total 30 sequences). In three out of five plants (#4-2-1, #4-2-4 and #4-3-3) without detectable TRSV, editing events were found. Still, these plants seem to be chimeric, because in addition to edited sequences with different numbers of deleted nucleotides (from four to nine) also wildtype PDS sequences were found. Two TRSV-free plants (#4-1-1 and #4-2-3) showed no editing.

TABLE 9
Summary of Nicotiana benthamiana PDS sequence
analysis in seedlings of F1 generation after
TRSV-mediated gene editing (individual plants).

| | | Sequence pattern | | | |
|---|---|---|---|---|---|
| | | Editing | | Total | |
| F1 plant line | TRSV | (deletion) | Wild type | number | % edited |
| #4-1-3 | stable | 1 | 5 | 6 | 20 |
| #4-1-12 | unstable | 0 | 5 | 5 | 0 |
| #4-1-1 | n.d. | 0 | 10 | 10 | 0 |
| #4-2-1 | unstable | 0 | 9 | 9 | 0 |
| #4-2-2 | stable | 4 | 4 | 8 | 50 |
| #4-2-1 | n.d. | 1 | 5 | 6 | 20 |
| #4-2-3 | n.d. | 0 | 8 | 8 | 0 |
| #4-2-4 | n.d. | 1 | 10 | 11 | 10 |
| #4-3-1 | stable | 0 | 5 | 5 | 0 |
| #4-3-8 | unstable | 0 | 8 | 8 | 0 |
| #4-3-10 | unstable | 0 | 8 | 8 | 0 |
| #4-3-3 | n.d. | 2 | 6 | 8 | 25 |

Stable: TRSV vector containing gRNA detected;
unstable: TRSV vector with gRNA deletion detected;
n.d.: no TRSV detected

Our data show that in Cas9-transgenic N. benthamiana plants editing events are transferred to the next generation also in the absence of TRSV vector. RT-PCR and editing analysis reveal editing in individual plants, in which TRSV is not detectable anymore.

### Example 10

### TRSV-Mediated Gene Editing in Developed Plants of Soybean

Stable Cas9 expressing transgenic soybean ‘Fayette’ plants transformed with pNMD34661 construct were inoculated with TRSV particles using rubbing of leaves. Viral particles were produced in Nicotiana benthamiana plants as described in Example 8. For this purpose, the following constructs were used: 1) pNMD36171 and pNMD45661 (TRSV vector with GmFT2a_SP1_gRNA), 2) pNMD36171 and pNMD47681 (TRSV vector with Gm_D7_PDS18_gRNA). The harvested leaf material containing viral particles was frozen in liquid nitrogen and ground using mortar and pestle; 2 g aliquots were stored at −80° C. For rubbing, 2 g of ground leaf material were extracted with 10 ml of 10 mM sodium phosphate buffer, pH 7.0. Plant sap preparations were mixed with silicon carbide F800 particles and rubbed into lowest trifoliate leaves of soybean plants. The sampling of the material from systemic leaves was done at 16 and 36 dpr (FIG. 15). Collected material was tested for gene editing events. Genomic DNA isolation, PCR amplification of the target sequence with D7-PDS18_18_PCR_f and D7-PDS18_18_PCR_r oligos specific for GmPDS18 gene, subcloning of PCR fragments into pJET1.2 vector, sequencing of individual plasmid clones and sequence analysis were performed as described in Example 8. At 16 dpr, no editing events were found in the control samples 1-1 and 1-2 transfected with TRSV-GmFT2a_SP1_gRNA vector (Table 10). In contrast, for the sample 2-3 (TRSV-Gm_D7_PDS18_gRNA), 17 out of 23 sequences contained editing events: 15 sequences had various deletions; 2 sequences contained single-nucleotide insertions. Again, no editing events were found in the control sample at 36 dpr (Table 10). In contrast, for the sample 2-3 (TRSV-Gm_D7_PDS18_gRNA), 23 out of 25 sequences contained editing events: 19 sequences with insertions and 4 sequences with deletions.

These data prove that gene editing in Cas9-transgenic soybean can be achieved by direct infection of developed plants with TRSV viral particles carrying target-specific gRNA.

TABLE 10
Gene editing in Cas9-transgenic soybean plants infected with TRSV particles
using rubbing of leaves: sequence patterns of individual clones.

| | | | | | |
|---|---|---|---|---|---|
| | | | | | Total |
| | | | Sequence | Sequences | number of |
| Construct | Sample | Sequence | pattern | with pattern | sequences |

16 days post rubbing

| | | | | | |
|---|---|---|---|---|---|
| TRSV- | 1-1 | GmPDS18 | 85M | 28 | 28 |
| GmFT2a_SP1 | 1-2 | GmPDS18 | 85M | 32 | 32 |
| TRSV- | 2-3 | GmPDS18 | 85M | 6 | 23 |
| gRNA_D7_PDS18 | | | 44M-1D-40M | 1 | |
| | | | 42M-3D-40M | 3 | |
| | | | 41M-4D-40M | 1 | |
| | | | 40M-5D-40M | 2 | |
| | | | 39M-6D-40M | 2 | |
| | | | 40M-4D-41M | 3 | |
| | | | 39M 5D-41M | 2 | |
| | | | 30M-16D-39M | 1 | |
| | | | 44M-1I-41M | 2 | |

36 days post rubbing

| | | | | | |
|---|---|---|---|---|---|
| TRSV- | 1-2 | GmPDS18 | 85M | 30 | 30 |
| GmFT2a_SP1 | | | | | |
| TRSV- | 2-3 | GmPDS18 | 85M | 2 | 25 |
| gRNA_D7_PDS18 | | | 44M-1D-40M | 4 | |
| | | | 42M-3D-40M | 8 | |
| | | | 44M-3D-37M | 2 | |
| | | | 40M-5D-40M | 3 | |
| | | | 39M-5D-41M | 1 | |
| | | | 39M-6D-40M | 3 | |
| | | | 38M-6D-41M | 1 | |
| | | | 37M-7D-41M | 1 | |
| | | | 45M-1I-40M | 4 | |

M: nucleotides matching with Reference Sequence;
D: deleted nucleotides;
I: inserted nucleotides

### REFERENCES

Cai, Y., Chen, L., Liu, X., Guo, C., Sun, S., Wu, C., Jiang, B., Han, T., Hou, W. (2018) CRISPR/Cas9-mediated targeted mutagenesis of GmFT2a delays flowering time in soya bean. Plant Biotechnol J 16(1): 175-185.
Du, H., Zeng, X., Zhao, M., Cui, X., Wang, Q., Yang, H., Cheng, H., Yu, D. (2015) Efficient targeted mutagenesis in soybean by TALENs and CRISPR/Cas9. J Biotechnol 217: 90-97.
Horsch R B, et al. (1985) A simple and general method for transferring genes into plants. Science 227(4691): 1229-1231.

### Mali P, Yang L, Esvelt K M, Aach J, Guell M, DiCarlo J E, Norville J E, Church G M (2013) RNA-Guided Human Genome Engineering via Cas9. Science 339 (6121): 823-826.

Olhoft, P. M., Flagel, L. E., Donovan, C. M., Somers, D. A. (2003) Efficient soybean transformation using hygromycin B selection in the cotyledonary-node method. Planta 216(5): 723-735.
Weber, E., Engler, C., Gruetzner, R., Werner, S., Marillonnet, S. (2011) A modular cloning system for standardized assembly of multigene constructs. PloS one 6(2): e16765.
Zhao, F., Lim, S., Igori, D., Yoo, R. H., Kwon, S. Y., Moon, J. S. (2016) Development of tobacco ringspot virus-based vectors for foreign gene expression and virus-induced gene silencing in a variety of plants. Virology 492: 166-178.

The content of European patent application No. 19 176 284.8, filed on May 23, 2019 is incorporated herein by reference including description, claims and figures.

### Nucleotide Sequences

SEQ ID NO: 1 Nucleotide sequence of cDNA copy of TRSV RNA1
SEQ ID NO: 2 Nucleotide sequence of cDNA copy of TRSV RNA2 (GenBank: KJ556850)
SEQ ID NO: 3 Nucleotide sequence of T-DNA region of pNMD36170
SEQ ID NO: 4 Nucleotide sequence of T-DNA region of pNMD36180
SEQ ID NO: 5 Nucleotide sequence of T-DNA region of pNMD43050
SEQ ID NO: 6 Nucleotide sequence of Nicotiana benthamiana phytoene desaturase (NbPDS) cDNA (GenBank: DQ469932.1)
SEQ ID NO: 7 Nucleotide sequence of T-DNA region of pNMD42330
SEQ ID NO: 8 Nucleotide sequence of T-DNA region of pNMD43741
SEQ ID NO: 9 Nucleotide sequence of soybean phytoene desaturase (GmPDS1) cDNA (NM_001249840.2)
SEQ ID NO: 10 Nucleotide sequence of T-DNA region of pNMD45660
SEQ ID NO: 11 Nucleotide sequence (genomic) of Flowering Locus T 2a (FT2a) gene from soybean (Glyma16g26660)
SEQ ID NO: 12 Nucleotide sequence of GmFT2a_SP1 gRNA (target sequence)
SEQ ID NO: 13 Nucleotide sequence of gRNA scaffold
SEQ ID NO: 14 Nucleotide sequence of T-DNA region of pNMD45680
SEQ ID NO: 15 Nucleotide sequence of NbPDS_Hpa1 gRNA (target sequence)
SEQ ID NO: 16 Nucleotide sequence of Nicotiana benthamiana phytoene desaturase (PDS) mRNA, complete cds
SEQ ID NO: 17 Nucleotide sequence of T-DNA region of pNMD46661
SEQ ID NO 18: Nucleotide sequence of Arabidopsis U6 promoter
SEQ ID NO 19: Nucleotide sequence of T-DNA region of pNMD47681
SEQ ID NO: 20 Nucleotide sequence (genomic) of phytoene desaturase gene GmPDS11 (Glyma.11G253000) from soybean
SEQ ID NO: 21 Nucleotide sequence (genomic) of phytoene desaturase gene GmPDS18 (Glyma.18G003900) from soybean
SEQ ID NO: 22 Nucleotide sequence of Gm_D7_PDS18 gRNA (target sequence)
SEQ ID NO: 23 Nucleotide sequence of T-DNA region of pNMD27570
SEQ ID NO: 24 Coding sequence of Cas9 endonuclease gene from Streptococcus pyogenes codon-optimized for Arabidopsis
SEQ ID NO: 25 Nucleotide sequence of T-DNA region of pNMD34661
SEQ ID NO: 26 Nucleotide sequence (genomic) of Nicotiana benthamiana phytoene desaturase gene NbPDS3a (Niben101Scf01283g02002.1)
SEQ ID NO: 27 Nucleotide sequence (genomic) of Nicotiana benthamiana phytoene desaturase gene NbPDS3b (Niben101Scf14708g00023.1)
SEQ ID NO: 28 Reference sequence 1 (fragment of Nicotiana benthamiana phytoene desaturase gene NbPDS3a (Niben101Scf01283g02002.1; SEQ ID NO: 26), nucleotide position 1201-1259)
SEQ ID NO: 29 Reference sequence 2 (fragment of Nicotiana benthamiana phytoene desaturase gene NbPDS3a (Niben101Scf01283g02002.1; SEQ ID NO: 26), nucleotide position 1214-1292)
SEQ ID NO: 30 Reference sequence 3 (fragment of Nicotiana benthamiana phytoene desaturase gene NbPDS3b (Niben101Scf14708g00023.1; SEQ ID NO: 26), nucleotide position 1260-1338)
SEQ ID NO: 31 Reference sequence 4 (fragment of soybean phytoene desaturase gene GmPDS11 (Glyma.11G253000; SEQ ID NO: 20), nucleotide position 1334-1418)
SEQ ID NO: 32 Reference sequence 5 (fragment of soybean phytoene desaturase gene GmPDS18 (Glyma.18G003900; SEQ ID NO: 21), nucleotide position 1173-1257)
SEQ ID NO: 33 oligonucleotide
SEQ ID NO: 34 oligonucleotide
SEQ ID NO: 35 oligonucleotide
SEQ ID NO: 36 oligonucleotide
SEQ ID NO: 37 oligonucleotide
SEQ ID NO: 38 oligonucleotide
SEQ ID NO: 39 oligonucleotide
SEQ ID NO: 40 oligonucleotide
SEQ ID NO: 41 oligonucleotide
SEQ ID NO: 42 oligonucleotide

1. A plus-sense single-stranded RNA viral RNA molecule, comprising a segment encoding a movement protein, a segment encoding a coat protein and a segment that comprises a guide RNA (gRNA), wherein said RNA molecule can be translated, in infected cells, to a polyprotein comprising the movement protein and the coat protein; or a complementary RNA thereof.

2. An RNA molecule comprising
(i) optionally a segment encoding a picornaviral P2A protein,
(ii) a segment encoding a picornaviral movement protein,
(iii) a segment encoding a picornaviral coat protein, and
(iv) a segment comprising a guide RNA (gRNA), preferably a single guide RNA (sgRNA);
or a complementary RNA thereof.

3. The RNA molecule according to claim 1, comprising, preferably in this order in 5′ to 3′-direction,
(i) optionally a segment encoding a TRSV (tobacco ring spot virus) P2A protein,
(ii) a segment encoding a TRSV movement protein,
(iii) a segment encoding a TRSV coat protein, and
(iv) a segment comprising a guide RNA (gRNA), preferably a single guide RNA (sgRNA);
or a complementary RNA thereof.

4. The RNA molecule according to claim 1, wherein said gRNA comprises a guide sequence linked to a direct repeat sequence.

5. The RNA molecule according to claim 1, wherein said gRNA is capable of forming a complex with a CRISPR nuclease capable of cleaving double-stranded DNA such as Cas9, optionally in the presence of a transactivating RNA.

6. The RNA molecule according to claim 4, wherein said gRNA is a single guide RNA (sgRNA) comprising a transactivating RNA in one RNA molecule.

7. The RNA molecule according to claim 1, wherein the RNA molecule further encodes a protein of interest to be expressed in a plant host, wherein the protein of interest is preferably a CRISPR effector protein capable of forming a complex with the gRNA.

8. A DNA molecule, DNA construct or vector encoding the RNA molecule of claim 1.

9. An Agrobacterium cell containing the DNA molecule according to claim 8, preferably containing a plasmid comprising in T-DNA the construct of claim 8.

10. A process of sequence-specifically affecting a target DNA, such as gene editing, in a plant or a plant cell, comprising the following steps:
(i) transfecting said plant with the RNA molecule of claim 1 or with a DNA molecule encoding the RNA molecule, and
(ii) transfecting said plant with a vector encoding proteins necessary for replicating said RNA molecule,
wherein said gRNA is capable of hybridizing with said target DNA and wherein steps (i) and (ii) can be performed in any order or simultaneously.

11. The process according to claim 10, further comprising providing said plant or said plant cell with an effector protein capable of forming a complex with the gRNA of said RNA molecule and with a target nucleic acid, preferably said target DNA, in said plant or plant cell, preferably wherein said RNA molecule encodes said effector protein and comprises said gRNA.

12. The process according to claim 10, wherein the effector protein is Cas9 or Cpf1 or a variant thereof having a nuclease activity removed by mutation.

13. A kit comprising
(a) the DNA molecule, DNA construct or vector according to claim 8, and
(b) a second DNA molecule or vector encoding proteins necessary for replicating and expressing the RNA molecule encoded by the DNA molecule, DNA construct or vector according to claim 8.

14. A process of producing a plus-sense single-stranded picornaviral RNA molecule in cells of a plant or plant cells, said RNA molecule comprising a segment encoding a movement protein, a segment encoding a coat protein and a segment that comprises a guide RNA (gRNA), wherein said RNA molecule can be translated, in infected cells, to a polyprotein comprising the movement protein and the coat protein,
said process comprising providing a DNA molecule encoding the RNA molecule into said cells of said plant or said plant cells.

15. A process of infecting a plant with a genetically-modified picornavirus, comprising
(A) providing, e.g. Agrobacterium-mediated, a first plant with a DNA molecule according to claim 8 encoding said RNA molecule for expressing said RNA molecule in cells of said first plant, and
(B) infecting said plant to be infected with said RNA molecule expressed in the first plant.

16. The process according to claim 15, further comprising collecting cell sap from said first plant, said sap containing said RNA molecule, and step (B) comprising rubbing or spraying said sap onto leaves optionally using an abrasive.

17. A plant, a plant tissue such as callus or shoot, a plant seed, or a plant cell containing the RNA molecule according to claim 1, or containing a DNA molecule, DNA construct or vector encoding the RNA molecule.

18. A plus-sense single-stranded RNA viral RNA molecule, wherein the RNA molecule is an RNA2 of a segmented nepoviral RNA comprising an inserted gRNA sequence; or a complementary RNA thereof.

19. A plus-sense single-stranded RNA viral RNA molecule according to claim 18, wherein the RNA molecule is an RNA2 of a tobacco ringspot virus (TRSV) comprising an inserted gRNA; or a complementary RNA thereof.
